(19) United States
(12) Patent Application Publication
(10) Pub. No.: US 2007/0072815 A1
(43) Pub. Date: Mar. 29, 2007

(54) Methods and kits to increase the efficiency of oligonucleotide-directed nucleic acid sequence alteration

(76) Inventors: Eric B. Kmiec, Landenberg, PA (US); Hetal Parekh-Olmedo, Mickleton, NJ (US); Luciana Ferrara, Torino (IT); Erin Brachman, Peakskill, NY (US)

Correspondence Address: Basil S. Krikelis;Citizens Bank Center, Suite 1800, 919 N. Market Street, Wilmington, DE, 19801-3033, US

(21) Appl. No.: 11/120,810

(22) Filed: May 3, 2005

Related U.S. Application Data
(60) Provisional application No. 60/568,339, filed on May 4, 2004.
(60) Provisional application No. 60/575,569, filed on May 27, 2004.
(60) Provisional application No. 60/634,584, filed on Dec. 8, 2004.

(51) Int. Cl. A61K 48/00; A61K 31/7072; A61K 31/7048; A61K 31/4745; A61K 31/4412; A61K 31/255; A61K 31/19

(52) U.S. Cl. 514/44; 514/27; 514/283; 514/49; 514/557; 514/575; 514/348; 514/517

(57) ABSTRACT

Methods, kits and cell lines are presented for effecting oligonucleotide-directed genetic alteration at a specific locus in a target DNA molecule in a population of cells at increased efficiency.

### CROSS-REFERENCE TO RELATED APPLICATIONS

This application claims the benefit of U.S. Provisional Application Ser. No. 60/568,339, filed May 4, 2004; U.S. Provisional Application Ser. No. 60/575,569, filed May 27, 2004; U.S. Provisional Application Ser. No. 60/634,584, filed Dec. 8, 2004, the contents of which are incorporated by reference.

### RELATED FEDERALLY SPONSORED RESEARCH

The work described in this application was sponsored by the National Institute of Health (NIH) under Contract No. R01CA89325.

### FIELD OF THE INVENTION

The invention relates to oligonucleotide-directed alteration of nucleic acid sequences.

### BACKGROUND OF THE INVENTION

A number of methods have been developed to alter specific nucleotides within both isolated DNA molecules and DNA present within intact cells of bacteria, plants, fungi and animals, including humans.

In one approach, genomic sequences are targeted for alteration by homologous recombination using duplex fragments. The duplex fragments are large, having several hundred basepairs. See, e.g., Kunzelmann et al., Gene Ther. (1996) 3:859-867.

In another approach, oligonucleotides are used to effect targeted genetic changes. In early experiments, oligonucleotide-directed sequence changes were typically effected in yeast, Moerschell et al., Proc. Natl. Acad. Sci. (USA)(1988) 85:524 and Yamamoto et al., Yeast 8:935 (1992), which among eukaryotes are known to have high recombinogenic activity, although one series of experiments was attempted in human cells, Campbell et al., The New Biologist (1989) 1: 223-227.

More recently, a number of different types of polynucleotides and oligonucleotides have been described that permit targeted alteration of genetic material in cells of higher eukaryotes, including (i) triplex-forming oligonucleotides; (ii) chimeric RNA-DNA oligonucleotides that are internally duplexed, notably in the region containing the nucleotide that directs the sequence alteration; and (iii) terminally modified single-stranded oligonucleotides having an internally unduplexed DNA domain and modified ends. Sequence-altering triplexing oligonucleotides are described, for example, in U.S. Pat. Nos. 6,303,376, 5,962,426, and 5,776,744.

Triplex-forming oligonucleotides require a structural domain that binds to a DNA helical duplex through Hoogsteen interactions between the major groove of the DNA duplex and the oligonucleotide. The binding domain must typically target polypurine or polypyrimidine tracts. These sequence requirements limit the usefulness of triplex-forming oligonucleotides for targeted sequence alteration, requiring that the target sequence to be modified be situated in proximity to such polypurine or polypyrimidine tract. Triplex-forming oligonucleotides may also require an additional DNA reactive moiety, such as psoralen, to be covalently linked to the oligonucleotide, in order to stabilize the interactions between the triplex-forming domain of the oligonucleotide and the DNA double helix if the Hoogsteen interactions from the oligonucleotide/target base composition are insufficient. See, e.g., U.S. Pat. No. 5,422,251. Such DNA-reactive moieties can, however, be indiscriminately mutagenic.

In more recent work with sequence-altering triplexing oligonucleotides, the triplex-forming domain is linked or tethered to a domain that effects targeted alteration, Culver et al., Nat. Biotechnology (1999) 17:989-93, relaxing somewhat the permissible distance between target sequence and polypurine/polypyrimidine stretch.

Internally duplexed, hairpin- and double-hairpin-containing chimeric RNA-DNA oligonucleotides are described, inter alia, in U.S. Pat. Nos. 6,573,046; 5,888,983; 5,871,984; 5,795,972; 5,780,296; 5,760,012; 5,756,325; 5,731,181, and 5,565,350. Such chimeric RNA-DNA oligonucleotides are reportedly capable of directing targeted alteration of single base pairs, as well as introducing frameshift alterations, in cells and cell-free extracts from a variety of host organisms, including bacteria, fungi, plants and animals. The oligonucleotides are reportedly able to operate on almost any target sequence.

Such chimeric molecules have significant structural requirements, however, including a requirement for both ribonucleotides and deoxyribonucleotides, and typically also a requirement that the oligonucleotide adopt a double-hairpin conformation. Even when such double hairpins are not required, however, significant structural constraints remain.

Single-stranded oligonucleotides having modified ends and an internally unduplexed DNA domain that directs sequence alteration are described in copending international patent applications published as WO 03/027265; WO 02/10364; WO 01/92512; WO 01/87914; and WO 01/73002, as well as in U.S. Pat. Nos. 6,479,292 and 6,271,360, the disclosures of which are incorporated herein by reference in their entireties. “Gene alteration” is the process in which a single base mutation is altered within the context of the chromosome using modified single stranded oligonucleotides to direct the reaction. The mechanism by which the oligonucleotides act is not well understood but the pathway likely includes a DNA pairing step and a DNA repairing phase. See Brachman and Kmiec, Curr. Opin. Mol. Ther. (2002) 4:171-76.

These single-stranded oligonucleotides have fewer structural requirements than chimeric oligonucleotides and are capable of directing sequence alteration, including introduction of frameshift mutations, in cells and cell-free extracts from a variety of host organisms, including bacteria, fungi, plants and animals, in episomal and in chromosomal targets, often at alteration efficiencies that exceed those observed with hairpin-containing, internally duplexed, chimeric oligonucleotides.

The usefulness of oligonucleotide-directed nucleic acid sequence alteration—as a means, for example, of manipulating cloned DNA, of generating agricultural products with enhanced traits, of generating cellular models for laboratory use, or of generating animal models or animals with desired traits—is affected by its frequency. Increased efficiency reduces the effort and expense required to obtain a cell with the desired sequence alteration by reducing the number of target cells that must be screened before finding a cell carrying the desired alteration. The usefulness of oligonucleotide-directed nucleic acid sequence alteration as an ex vivo or in vivo therapeutic method would also be enhanced by increasing its efficiency, since it is likely that a minimum threshold of target cells must be altered in order to give a clinically relevant therapeutic benefit for any given genetic disease.

A need exists, therefore, for methods to increase the efficiency of targeted alteration of genetic material.

### SUMMARY OF THE INVENTION

The present invention provides methods and kits to increase the efficiency of oligonucleotide-directed nucleic acid sequence alteration (ODSA).

In one embodiment, the present invention provides methods for increasing the efficiency of ODSA by modulating the cell cycle of cells within a population of target cells.

In another embodiment, the present invention provides methods for increasing the efficiency of ODSA by inducing DNA repair pathways within a population of target cells.

In yet another embodiment, the present invention provides methods for increasing the efficiency of ODSA by inducing DNA damage within a population of target cells.

In a further embodiment, the present invention provides methods for increasing the efficiency of ODSA by inducing homologous recombination pathways within a population of target cells.

In another embodiment, the present invention provides methods for increasing the efficiency of ODSA by treating a population of target cells with hydroxyurea (HU).

In another embodiment, the present invention provides methods for increasing the efficiency of ODSA by treating a population of target cells with etoposide (VP16).

In another embodiment, the present invention provides methods for increasing the efficiency of ODSA by treating a population of target cells with thymidine.

In another embodiment, the present invention provides methods for increasing the efficiency of ODSA by treating a population of target cells with methyl methanesulfonate (MMS).

In another embodiment, the present invention provides methods for increasing the efficiency of ODSA by treating a population of target cells with valproic acid (VPA).

In another embodiment, the present invention provides methods for increasing the efficiency of ODSA by treating a population of target cells with camptothecin (CPT).

In another embodiment, the present invention provides methods for increasing the efficiency of ODSA by treating a population of target cells with dideoxycytidine (ddC).

In another embodiment, the present invention provides methods for increasing the efficiency of ODSA by treating a population of target cells with caffeine.

In another embodiment, the present invention provides methods for increasing the efficiency of ODSA by treating a population of target cells with an agent selected from the group consisting of thymidine, HU, VP16, VPA, MMS, camptothecin, ddC and caffeine.

In yet another embodiment, the present invention provides methods for increasing the efficiency of ODSA by treating a population of target cells with a plurality of agents selected from the group consisting of thymidine, HU, VP16, VPA, MMS, camptothecin, ddC and caffeine.

In another aspect, the present invention provides kits for performing the aforementioned methods.

In yet another aspect, the present invention provides cell lines for use in performing the aforementioned methods, and/or for inclusion in the aforementioned kits.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1A shows the structure of an integrative cassette comprising a mutant gene encoding green fluorescent protein (EGFP-N3 (mutant)), as well as the wild type counterpart sequence (EGFP-N3 (wt)), used to create the DLD-1-derived mammalian cell line designated DLD-1-1, as described in copending U.S. patent application Ser. No. 10/986,418, filed Nov. 10, 2004 (“Mammalian Cell Lines for Detecting, Monitoring, and Optimizing Oligonucleotide-Mediated Chromosomal Sequence Alteration”), the disclosure of which is incorporated herein by reference in its entirety.

FIG. 1B shows the relevant segment of the sequence of mutant and wild type eGFP alleles, and the sequences of a single-stranded oligonucleotides used to correct the eGFP mutation (EGFP3S/72NT) and a non-specific control oligonucleotide (Hyg3S/74NT). Asterisks represent phosphorothioate linkages.

FIG. 2 presents a protocol for sequence alteration (“gene alteration”) in engineered DLD-1-1 cells, according to the present invention.

FIG. 3 presents fluorescence activated cell sorting (FACS) data demonstrating an increased proportion of cells expressing high levels of GFP in DLD-1-1 cells treated with EGFP3S/72NT compared to untreated cells.

FIG. 4 presents FACS data showing the number of cells in populations of DLD-1-1 cultures, as a function of DNA content, at various times after release from cell cycle arrest, as effected by serum starvation and treatment with mimosine. FIG. 4 also shows, in tabular form, the distribution of cells in the cell cycle, and the average “correction efficiency” (“C.E.”) when the aforementioned populations of cells are treated with EGFP3S/72NT. Asynchronous cells are those not subjected to cell cycle arrest but otherwise identically treated.

FIG. 5 presents a pulsed-field gel of DNA from DLD-1-1 cells that have been treated with 0.3, 1 or 5 mM HU, or 0.5, 1 or 3 μM VP16. “C” a control sample from cells that were not exposed to HU or VP16, and “M” represents a lane of size markers (notably 745, 785, 815 and 1120+1100 Kbp).

FIG. 6 presents the correction efficiency as a percentage of the number cells treated, and cell viability, as a function of the dose of HU and VP16 used to treat DLD-1-1 cells. When correction efficiency is presented “as a percentage” herein it refers to the percentage of all treated cells that exhibit the corrected phenotype after treatment, unless otherwise indicated.

FIG. 7 presents time courses for treatment of DLD-1-1 cells with HU and VP16 in ODSA experiments.

FIG. 8 presents the FACS data showing the distribution of DLD-1-1 cells in the cell cycle after no treatment, treatment with 1 mM HU for 24 hours or treatment with 3 μM VP16 for 24 hours. Tables present the percentage of cells in S-phase, based on the FACS data, and results of BrdU incorporation experiments for each population of cells.

FIG. 9A presents FACS data showing the fraction of cells in each phase of the cell cycle for populations of DLD-1-1 cells either unsynchronized or synchronized using a double thymidine block procedure, with the percentage of cells in S-phase presented beneath each plot.

FIG. 9B presents the correction efficiency in ODSA experiment, performed on synchronized (dark bars) and unsynchronized (light bars) DLD-1-1 cultures, as a function of their treatment with 1 mM HU, 3 μM VP16 or 10 mM Thymidine. Control cells were not treated with any of the listed agents but were otherwise identically treated.

FIG. 10 presents a pulsed field gel illustrating DNA damage caused by treatment of DLD-1-1 cells with 0.75 μM bleomycin or 0.2 μM MMS compared with DNA from untreated cells.

FIG. 11A presents the percentage of DLD-1-1 cells expressing GFP in populations treated with 10 μg EGFP3S/72NT with or without 0.2 μM MMS, and in an untreated population. The data are also presented in the table below the plot, along with cell death data.

FIG. 11B presents the percentage of DLD-1-1 cells expressing GFP in populations treated with 10 μg EGFP3S/72NT and: nothing; 0.2 μM MMS; 0.2 μM MMS+4 mM caffeine; 4 mM caffeine. The data are also presented in the table below the plot, along with cell death data. In this and other figures herein “uM” is used synonymously with “μM” (micromolar).

FIG. 12 presents correction efficiency (as a percentage) in a series of ODSA experiments as a function of the dosage of wortmannin (WM), alone or in combination with 30 nM CPT.

FIG. 13A presents correction efficiency (as a percentage) in a series of ODSA experiments as a function of the dosage of ddC.

FIG. 13B presents correction efficiency (as a percentage) in a series of ODSA experiments as a function of treatment with 500 μM ddC, without 4 mM caffeine, or with 4 mM caffeine added either before (“prior”) or after (“recovery”) electroporation.

FIG. 13C presents correction efficiency (as a percentage) in a series of ODSA experiments as a function of treatment with 500 μM ddC, without 1 mM vanillin, or with 1 mM vanillin added either before (“prior”) or after (“recovery”) electroporation.

FIG. 13D presents a time course of correction efficiency (as a percentage) in a series of ODSA experiments as a function of treatment with 500 μM ddC, either without caffeine, or with 4 mM caffeine added after electroporation for 12, 24 or 48 hours.

FIG. 14A presents BrdU incorporation (as a percentage of control) for DLD-1-1 cells as a function of time after treatment with 3 μM CPT.

FIG. 14B presents correction efficiency (relative to control) for DLD-1-1 cells as a function of time after treatment with 3 μM CPT.

FIG. 14C presents correction efficiency (as a percentage) in a series of ODSA experiments as a function of the dosage of CPT.

FIG. 14D presents correction efficiency (as a percentage) in a series of ODSA experiments as a function of treatment with CPT alone or in combination with other agents and related controls.

FIG. 15A presents GLA activity in Fabry's cells as a function of the sequence of oligonucleotides used in ODSA experiments on Fabry's cells, and the amount of each oligo used.

FIG. 15B presents correction efficiency (as a percentage) in Fabry's cells in a series of ODSA experiments as a function of treatment with HU, VP16, CPT, thymidine (thy), p7 and various combinations, permutations and dosages thereof.

FIG. 15C presents GLA activity in Fabry's cells as a function of treatment with HU, and dosages thereof, with the oligonucleotide being either 49T/pm or 49T/gg, or no oligonucleotide, as indicated, seven days after electroporation.

FIG. 15D presents GLA activity in Fabry's cells as a function of treatment with VPA, CPT, p7 and various combinations, permutations and dosages thereof, with the oligonucleotide being either 49T/pm or 49T/gg, or no oligonucleotide, as indicated.

FIG. 15E presents GLA activity in synchronized Fabry's cells as a function of treatment with combinations of HU (0.3, 1 or 3 mM), 500 μM ddC, 4 mM caffeine or 100 ng/ml trichostatin A (TSA), as indicated.

FIG. 16 presents a dose response curve for ddC stimulation of gene repair in DLD-1 cells exposed to various doses of 2′3′-dideoxycytidine (ddC) for 24 hrs prior to electroporation with oligonucleotide, with the percentage correction efficiency (C.E. (%)) determined 48 hrs later by the percent of fluorescent cells as a function of the correction of the eGFP gene; results are averaged over four experiments. The treatments demarcated by (*) are statistically significant with a p value of <0.05 relative to the no treatment control.

FIGS. 17A presents profiles of cell cycle under various indicated (24 hour) reaction conditions.

FIG. 17B presents profiles of BrdU incorporation under various indicated (24 hour) reaction conditions.

FIG. 18A demonstrates statistically insignificant effect of ddI on correction efficiency.

FIG. 18B demonstrates significantly insignificant effect of AraC on correction efficiency.

FIG. 18C demonstrates the effect on BrdU incorporation and correction efficiency, respectively, at various time points following release from AraC.

FIG. 18D also demonstrates the effect on BrdU incorporation and correction efficiency, respectively, at various time points following release from AraC.

FIG. 18E tabulates correction efficiencies at various time points after release from either AraC or Aphidicolin treatment, with the viability, total count of fluorescent cells, and the correction frequency (C.E.) presented.

FIG. 19A demonstrate that p53 blocks or suppresses gene repair activity stimulated by ddC, with FIG. 19A verifying expression of p53 by Western blot analysis of cell extracts prepared 24 hrs after the introduction of the expression construct.

FIG. 19B shows correction efficiency in the presence of the indicated p53 or control constructs. Asterisks indicate statistically significant differences from the control (empty expression construct) (p value of <0.05).

FIG. 20 demonstrates that caffeine but not vanillin knocks down correction induced by ddC, graphing the average of three experiments, with standard deviation presented. Samples showing a statistically significant difference from the controls (p value of <0.05) are denoted.

### DETAILED DESCRIPTION OF PREFERRED EMBODIMENTS

DNA oligonucleotides may be used to introduce single base changes into the genomes of prokaryotic and eukaryotic cells. See Liu et al., Nat. Rev. Genet. (2003) 4:679-689, the disclosure of which is incorporated herein by reference in its entirety. Our results show that cells grown under conditions where the process of DNA replication is arrested or elongated, or double-strand breaks (DSB) are induced, support a higher frequency of oligonucleotide-directed sequence alteration. As used herein, “oligonucleotide-directed sequence alteration” (ODSA) is synonymous with “oligonucleotide-mediated sequence alteration.” The frequency of oligonucleotide-directed sequence alterations is higher in cells that are in S-phase, and reducing the rate at which cells pass through S phase leads to an increased frequency of targeted gene alteration, perhaps due to the accumulation of double strand breaks and the activation of the homologous recombination pathway.

Our results also show that that the frequency of oligonucleotide-directed sequence alteration is higher in cells in which enzymatic activities that promote gene repair or gene editing are induced. Such enzymatic activities or DNA repair pathways include, but are not limited to, homologous recombination, mismatch repair, RAD51 and RAD52 mediated recombination, expression of lambda beta protein, and non-homologous end joining.

Mechanisms to induce such DNA repair pathways include damaging DNA, stalling cells during the cell cycle, and slowing the progress of cells though S-phase. Means to induce DNA damage include digestion with restriction enzymes, exposure to ionizing radiation, or exposure to cells to genotoxic agents (as discussed in greater detail infra). Means to stall cells in S-phase or otherwise increase the number of replication forks per genome include treatment of cells with HU, camptothecin or other agents.

Overall, our results show that the efficiency of ODSA can be influenced by the position of the target cells in the cell cycle and the activation of DNA damage response pathways. For example, we have observed that actively replicating mammalian cells passing through S-phase are more amenable to sequence alteration, and that the efficiency of sequence alteration is reproducibly enhanced by activation of the homologous recombination pathway in response to DNA damage. We have also observed that the replication and/or transcriptional state of the target gene can influence the alteration efficiency.

The methods, kits and cell lines of the present invention make use of these observations to provide a procedure for oligonucleotide-directed gene alteration that is more efficient and more reproducible than previous procedures. Such highly efficient gene alteration is essential to make oligonucleotide-directed gene alteration practically useful for methods for many purposes, such as ex vivo or in vivo gene therapy.

The methods of the present invention may increase the efficiency with which bacteria, plant, fungi and animal cells are altered by oligonucleotide-directed sequence alteration. In related aspects, the invention provides kits for effecting or facilitating practice of the methods of the present invention; mammalian cell lines for determining the efficiency of oligonucleotide-directed sequence alteration; and related business methods.

### Targeted Genomic DNA

The targeted genomic DNA can be normal, cellular chromosomal DNA; organellar DNA, such as mitochondrial or plastid DNA; or extrachromosomal DNA present in cells in different forms including, e.g., mammalian artificial chromosomes (MACs), PACs from P-1 vectors, yeast artificial chromosomes (YACs), bacterial artificial chromosomes (BACs), plant artificial chromosomes (PLACs), BIBACS, as well as episomal DNA, including episomal DNA from an exogenous source such as a plasmid or recombinant vector. Many of these artificial chromosome constructs containing human DNA can be obtained from a variety of sources, including, e.g., the Whitehead Institute, and are described, e.g., in Cohen et al., Nature 336: 698-701 (1993) and Chumakov, et al., Nature 377: 175-297 (1995).

The targeted nucleic acid site may be in a part of the DNA that is transcriptionally silent or transcriptionally active. The targeted site may be in any part of a gene including, for example, an exon, an intron, a promoter, an enhancer or a 3′- or 5′-untranslated region, and may be in an intergenic region.

### Sequence-Altering Oligonucleotides

In some embodiments, the sequence-altering oligonucleotide is designed to direct alteration of the transcribed strand of the target sequence; in other embodiments, the sequence-altering oligonucleotide is designed to direct alteration of the non-transcribed strand.

The level of gene alteration may also be affected by the position of the mismatched base pair (i.e. the target locus) within the sequence altering oligonucleotide. Highest efficiency gene alteration is obtained when the target locus is near the center of the correcting oligonucleotide, with approximately a two-fold reduction in efficiency when the target locus is located near the 3′ end of the oligo, and up to a 17-fold reduction when the target locus is located near the 5′ end of the oligo.

Alteration efficiency may also vary depending on whether the sequence altering oligonucleotide is designed to hybridize to the transcribed or the non-transcribed strand of the target gene, and in some cases hybridization to the non-transcribed strand gives higher alteration efficiency. In addition, experiments in which the orientation of a mutant eGFP gene relative to an SV40 origin of replication on an episome in COS1 cells is varied confirm the aforementioned transcription strand bias, and further showing that the lagging strand in DNA replication is a more efficient target for sequence alteration. These two effects, referred to herein as “transcription bias” and “replication bias,” may not be the same in other cells or at other genetic loci, however. The strand bias results suggest that oligonucleotides directed to both strands at a target locus should be tested to insure that the oligonucleotide giving the highest possible alteration efficiency is determined.

The sequence-altering oligonucleotide may be selected from any type of sequence-altering oligonucleotide known in the art, including (i) triplex-forming oligonucleotides; (ii) chimeric RNA-DNA oligonucleotides that are internally duplexed, notably in the region containing the nucleotide that directs the sequence alteration; and (iii) terminally modified single-stranded oligonucleotides having an internally unduplexed DNA domain and modified ends. See. e.g., Liu et al., J. Mol. Med. (2002) 80:620-28; Nakamura et al., Gene Therapy (12 Feb. 2004) 1-9; Bertoni et al., Hum. Mol. Genet. (2003) 12(10):1087-99; Alexeev et al., Gene Therapy (2002) 9:1667-75; Pierce et al., Gene Therapy (2003) 10:24-33; Suzuki et al., Int'l. J. Mol. Med. (2003) 12:109-14; Kren et al., DNA Repair (2003) 2:531-46; Goukassian et al., FASEB J. (Mar. 26, 2002), the disclosures of which are incorporated herein by reference in their entireties.

In methods of performing the function of effecting a desired sequence alteration at a nucleic acid target site within a cell according to the present invention, steps for effecting such alteration include, but are not limited to, treating cells with triplex-forming oligonucleotides, chimeric RNA-DNA oligonucleotides that are internally duplexed or terminally modified single-stranded oligonucleotides having an internally unduplexed DNA domain and modified ends.

Sequence-altering triplexing oligonucleotides useful in the methods, compositions, and kits of the present invention are described, for example, in U.S. Pat. Nos. 6,303,376, 5,962,426, and 5,776,744, the disclosures of which are incorporated herein by reference in their entireties. Bifunctional oligonucleotides having a triplex-forming domain linked or tethered to a domain that effects targeted alteration, useful in the methods, compositions, and kits of the present invention, are described in Culver et al., Nat. Biotechnology (1999) 17:989-93, the disclosure of which is incorporated herein by reference in its entirety.

Internally duplexed, hairpin- and double-hairpin-containing chimeric RNA-DNA oligonucleotides useful in the methods, compositions, and kits of the present invention are described, inter alia, in U.S. Pat. Nos. 6,573,046; 5,888,983; 5,871,984; 5,795,972; 5,780,296; 5,760,012; 5,756,325; 5,731,181 and 5,565,350, the disclosures of which are incorporated herein by reference in their entireties.

In some embodiments, the sequence-altering oligonucleotide is a single-stranded oligonucleotide having modified ends and an internally unduplexed DNA domain that directs sequence alteration. Such oligonucleotides are further described in copending international patent applications published as WO 03/027265; WO 02/10364; WO 01/92512; WO 01/87914; and WO 01/73002, as well as in U.S. Pat. Nos. 6,479,292 and 6,271,360, the disclosures of which are incorporated herein by reference in their entireties.

The “sequence-altering oligonucleotide” is designed to have the desired sequence at the locus in question (e.g. a mismatch relative to the base to be altered) and to have sequence complementary to the target DNA molecule on both sides (upstream and downstream) of the locus. “Sequence-altering,” as used herein, is not intended to imply any specific phenotypic effect of the desired alteration. Similarly, the phrase “gene alteration” is not intended to imply any specific resulting phenotype. The phrase “gene repair” is used synonymously with “gene alteration” herein. A sequence-altering oligonucleotide, and a gene alteration event, can involve introduction of any desired genetic alteration, including those that restore a function, disrupt a function, up-regulate or down-regulate gene expression, or effect any other alteration, whether giving rise to an altered phenotype or not. The phrase gene repair, as used herein, is not limited to “repair” in the sense of restoring the lost function of a gene, but instead refers generally to any desired gene alteration. Such alterations include introduction of nonsense, frameshift, missense or other mutations that may either increase or decrease the activity of a gene, or leave the resulting protein or gene activity unchanged. The term “correction” and the phrase “gene correction,” as used herein, are not intending to be limiting, but may be used for simplicity in instances where a mutant gene is being altered to restore a lost function, as in an assay to restore activity to a mutant green fluorescent protein or to treat a genetic disease.

The sequence-altering oligonucleotide can direct any kind of alteration, including, for example, deletion, insertion or replacement of 1, 2, 3 or more nucleotides in the target sequence. These altered nucleotides may be contiguous or non-contiguous with each other. Multiple alterations can be directed to a target site by a single oligonucleotide or by 1, 2, 3 or more separate oligonucleotides. In some embodiments, the multiple alterations are directed by a single oligonucleotide. In some embodiments, the multiple alterations are within 1 to 10 nucleotides of each other.

For example, the methods and kits of the invention can be used to produce “knock out” mutations by modification of specific amino acid codons to produce stop codons (e.g., a CAA codon specifying glutamine can be modified at a specific site to TAA; a AAG codon specifying lysine can be modified to TAG at a specific site; and a CGA codon for arginine can be modified to a TGA codon at a specific site). Such base pair changes will terminate the reading frame and produce a truncated protein shortened at the site of the stop codon, which truncated protein may be defective or have an altered function. Alternatively, frameshift additions or deletions can be directed at a specific sequence to interrupt the reading frame and produce a garbled downstream protein. Such stop or frameshift mutations can be introduced to determine the effect of knocking out the protein in either plant or animal cells.

The oligonucleotide-directed gene alteration methods and kits disclosed herein are well suited to effect therapeutic changes in many genetic diseases. According to the Human Gene Mutation Database (<http://archive.uwcm.ac.uk/uwcm/mg/hgmd0.html>), the great majority of known disease-causing genetic mutations can be classified as “micro-lesions,” i.e. missense, nonsense, splicing, regulatory, and small deletions, insertions and indels. <http://archive.uwcm.ac.uk/uwcm/mg/docs/hahaha.html>.

In typical gene repair embodiments, the sequence-altering oligonucleotide is 17-121 nucleotides in length and has an internally unduplexed domain (that is, a non-hairpin domain) of at least 8 contiguous deoxyribonucleotides. The oligonucleotide is fully complementary in sequence to the sequence of a first strand of the respective nucleic acid target, but for one or more mismatches as between the sequences of the oligonucleotide internally unduplexed deoxyribonucleotide domain and its complement on the target nucleic acid first strand. Each of the mismatches is positioned at least 8 nucleotides from each of the oligonucleotide's 5′ and 3′ termini. The oligonucleotide has at least one terminal modification.

In some embodiments, the at least one terminal modification may be selected from the group consisting of 2′-O-alkyl, such as 2′-O-methyl, residue; phosphorothioate internucleoside linkage; and locked nucleic acid (LNA) residue. The basic structural and functional characteristics of LNAs and related analogues are disclosed in various publications and patents, including WO 99/14226, WO 00/56748, WO 00/66604, WO 98/39352 and U.S. Pat. Nos. 6,043,060 and 6,268,490, the disclosures of which are incorporated herein by reference in their entireties. In some embodiments, the terminal modification comprises a plurality of adjacent phosphorothioate internucleoside linkages, such as three phosphorothioate linkages at the 3′ terminus of the oligonucleotide.

In some embodiments, a plurality of single-stranded oligonucleotides having modified ends and an internally unduplexed DNA domain that directs sequence alteration can be used to effect sequence alterations. Use of such plural oligonucleotides is described in copending U.S. patent application Ser. No. 10/623,107, filed Jul. 18, 2003 (“Targeted Nucleic Acid Sequence Alteration Using Plural Oligonucleotides”), the disclosure of which is incorporated herein by reference in its entirety.

The oligonucleotides used in the methods, compositions and kits of the invention can be introduced into cells or tissues by any technique known to one of skill in the art. Such techniques include, for example: electroporation; transfection; carrier-mediated delivery using, e.g., liposomes, aqueous-cored lipid vesicles, lipid nanospheres or polycations; naked nucleic acid insertion; particle bombardment and calcium phosphate precipitation.

In some embodiments, the oligonucleotides are introduced using electroporation, for example using a BTX ECM® 830 Square Wave electroporator. Electroporation may be carried out in a 4 mm gap cuvette using two 250V pulses, each 13 msec long, with a 1 second pulse interval. In other embodiments, electroporation is carried out using 1, 2, 3 or 10 pulses at 170, 250, 300, 600 or 2000V, each pulse lasting 10, 30, 70 or 99 msec. Electroporation may also be carried out in a 2 mm gap cuvette using 1, 2, 3 or 10 pulses at 225, 300, 480 or 500V, each pulse lasting 22, 99 or 1000 msec. One of skill in the art would recognize that the particular settings for electroporation may vary from experiment to experiment and are not critical aspects of the embodiments of the present invention.

In other embodiments transfection is performed with a liposomal transfer compound, for example, DOTAP (N-1-(2,3-Dioleoyloxy)propyl-N,N,N-trimethylammonium methylsulfate, Boehringer-Mannheim) or an equivalent, such as LIPOFECTIN®. In other embodiments, the transfection technique uses cationic lipids. In some embodiments, transfection is performed with Lipofectamine™ 2000 (Invitrogen Corporation, Carlsbad, Calif.). In still further embodiments, transfection is performed with FuGENE™ 6 (FG) (Roche Diagnostics Corp., Indianapolis, Ind., USA).

### Selectable Phenotype

In some embodiments of the methods and kits of the present invention, the sequence-altering oligonucleotide directs an alteration that produces a selectable phenotype. In other embodiments, the sequence-altering oligonucleotide directs an alteration that must be identified by screening, e.g., by determining the corresponding nucleic acid sequence or by assaying a non-selectable phenotype that is generated by the alteration event.

In some embodiments of the present invention, a second oligonucleotide is added to effect a sequence alteration at a second nucleic acid target site, the second sequence alteration conveniently conferring a selectable marker phenotype on the target cells that facilitates identification of cells harboring the desired sequence alteration at the first nucleic acid target site. Such embodiments are further discussed in co-pending U.S. patent application Ser. No. 10/681,074, filed Oct. 7, 2003 (“Methods and Compositions for Reducing Screening in Oligonucleotide-Directed Nucleic Acid Sequence Alteration”), the disclosure of which is incorporated herein by reference in its entirety.

In embodiments involving a selectable phenotype, the selectable phenotype chosen will depend on the host cell chosen and whether the selection is effected in vitro or in vivo. As is well known in the art, exemplary selectable phenotypes include, e.g., antibiotic or other chemical resistance, ability to use a nutrient source, expression of a fluorescent protein, presence of an epitope or resistance to an apoptotic signal.

The selectable phenotype chosen may be selectable based on preferential growth of a cell with the desired sequence alteration. Examples of such selectable phenotypes include, e.g., the ability to grow in the presence of a compound that either kills or prevents the growth of the cell such as an apoptotic signal or an antibiotic, the ability to grow in the absence of a nutrient that is required prior to the sequence alteration, or the ability to utilize a particular resource that is not usable prior to the sequence alteration.

The selectable phenotype may also be selected mechanically. Examples of phenotypes that may be selected mechanically include, e.g., expression of a fluorescent protein or a particular epitope. Mechanical selection may be by any means known to one of skill in the art including, e.g., fluorescence activated cell sorting (FACS) (directly in the case of a fluorescent protein or using a labeled antibody for an epitope), column chromatography, or using paramagnetic beads produced by, e.g., Miltenyi Biotec (Auburn, Calif., USA). Selection also does not require intact cells. For example, a single nucleotide change (SNP) in a nucleic acid molecule may be detected and isolated in vitro using methods such as are described in WO 03/027640, the disclosure of which is incorporated herein by reference in its entirety. In such cases, the sequence-altering oligonucleotide effects a change in the selected molecule.

In methods of performing the function of detecting the presence or absence of a selectable phenotype in target cells according to the present invention, steps for selecting include, but are not limited to, selecting for antibiotic or other chemical resistance, the ability to use a nutrient source, expression of a fluorescent protein, the presence of an epitope, resistance to an apoptotic signal, the ability to grow in the presence of a compound that typically either kills or prevents the growth of the cell such as an apoptotic signal or an antibiotic, the ability to grow in the absence of a nutrient that is required prior to the sequence alteration, the ability to utilize a particular resource that is not usable prior to the sequence alteration and expression of a fluorescent protein or a particular epitope.

### DLD-1-1 Mammalian Cell Test System

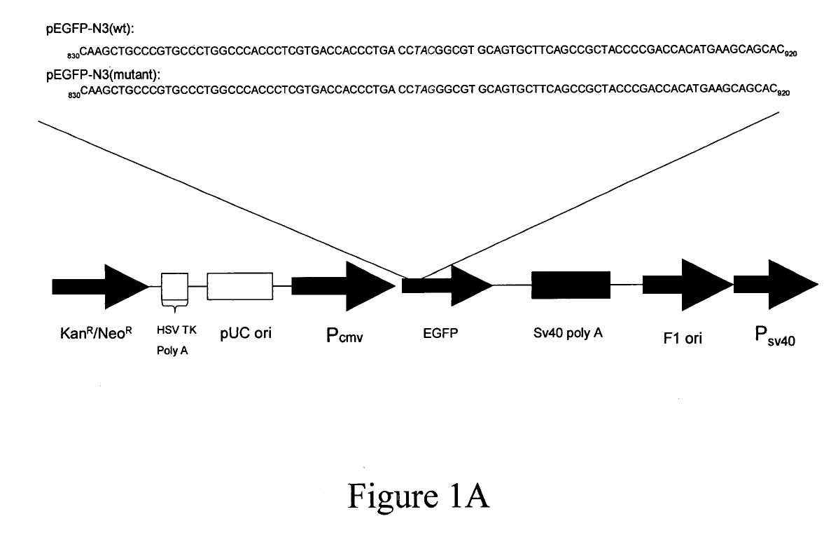

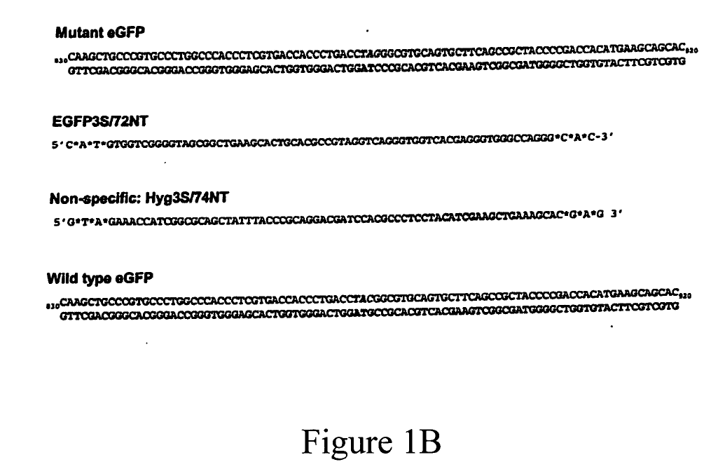

The mammalian cell line DLD-1-1, carrying a mutant version of the gene encoding green fluorescent protein (eGFP), is constructed as described in Example 1. This DLD-1-1 cell line is used as the experimental model system in the experiments described herein unless otherwise indicated. The genetic cassette carrying the mutant eGFP gene that is introduced into the parent DLD-1 cell line is shown at FIG. 1A, along with wild type sequence. The mutation at position 875 of the gene creates a premature stop codon (Y291X) that inactivates the green fluorescent protein. FIG. 1B shows the sequence altering oligonucleotide (EGFP3S/72NT), and the non-specific control oligonucleotide (Hyg3S/74NT), that are used in the experiments described herein (except where otherwise indicated).

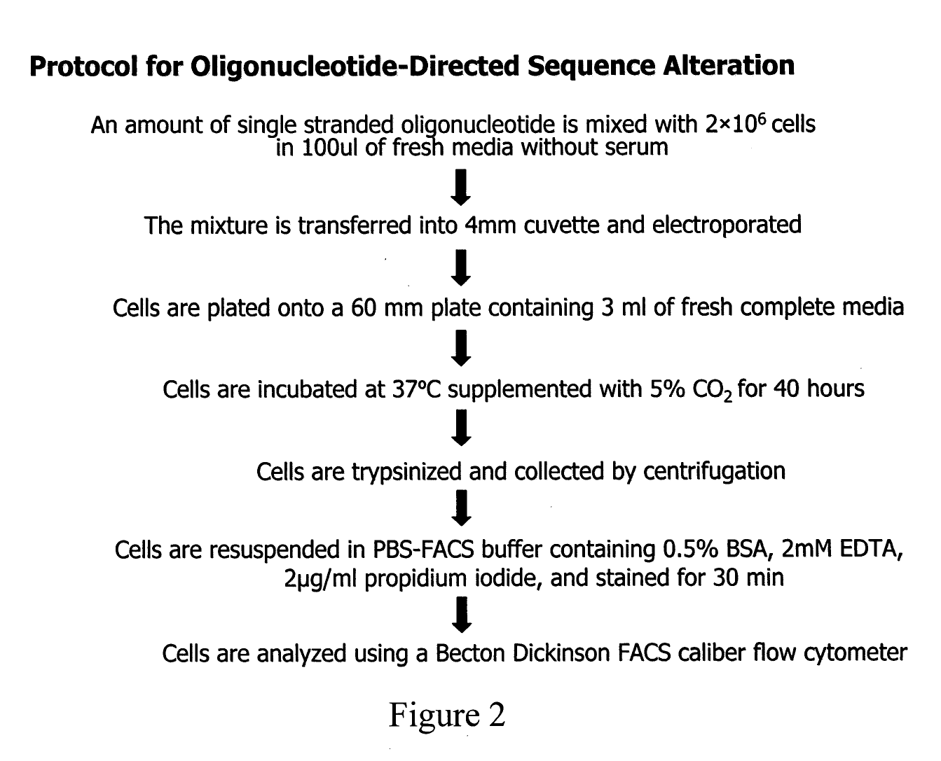

The general protocol for oligonucleotide-directed sequence alteration is presented schematically at FIG. 2. Additional details are provided in Example 1 and other examples. Many embodiments of the present invention include steps in addition to those listed in FIG. 2, including treatment steps before or after electroporation to increase the efficiency of sequence alteration. Some embodiments deviate from the listed steps or omit one or more of them.

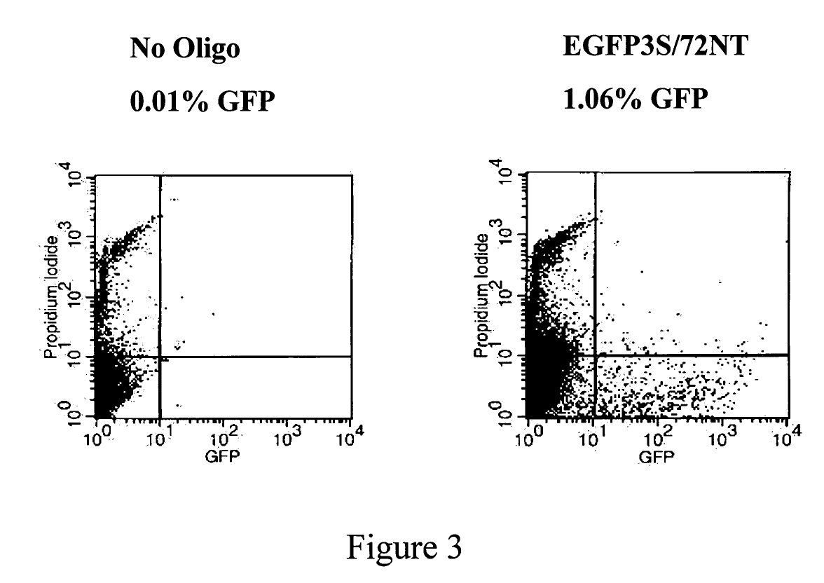

The utility of the DLD-1-1 experimental test system is illustrated at FIG. 3, where fluorescent activated cell sorting (FACS) data are presented for correction of the eGFP gene in 50,000 cells treated with 10 μg EGFP3S/72NT compared with 50,000 untreated cells, as discussed in more detail in Example 1. The fraction of cells in the lower-right quadrant, representing living cells with corrected eGFP genes, increases from 0.01% to over 1% when treated with EGFP3S/72NT.

An alternative model system to measure oligonucleotide-directed sequence alteration has been developed in the yeast Saccharomyces cerevisiae strain LSY678 (MATa leu 2-3, 112 trpl-1 ura 3-1 his 3-1, 15 ade2-1 can 1-100). The strain has integrated an HYGeGFP fusion gene target containing a single point mutation at base pair 137 in the coding region of the hygromycin gene, rendering it unable to confer resistance to the antibiotic. Oligonucleotide-directed sequence alteration can repair the mutation and restore hygromycin resistance. For example, in one experiment, LSY678 cells are synchronized with alpha factor and released, or synchronized with alpha factor and released into hydroxyurea (HU), prior to electroporation with a correcting oligonucleotide. The combination of alpha factor and HU increased correction efficiency 25-fold as compared to cells treated with neither agent, but only when oligonucleotide treatment is performed at a specific period of time after release from the G1/S border. See also U.S. patent application publication no. 20030207451.

### Cell Cycle Modulation

Although techniques for oligonucleotide-directed gene alteration have sometimes achieved remarkably high levels of nucleotide exchange, the frequency of gene repair in mammalian cells has been highly variable. The observed variability may be due, at least in part, to use of populations of target cells that are, on average, at different phases of the cell cycle. Many embodiments of the methods of the present invention use cell populations in which the phase of the cell cycle is modulated to increase the efficiency of sequence alteration.

In some embodiments of the present invention, cells that are to be subjected to sequence alteration are synchronized prior to treatment with alteration-inducing oligonucleotides. Synchronization, as used herein, refers to the treatment of a population of cells so as to increase the fraction of cells in any given phase of the cell cycle. A typical asynchronous population of cells is comprised of a mixture of cells in various phases of the cell cycle, such as S, M, G1 and G2-phases. Synchronization may be effected by treatments that arrest cells at a given point in the cell cycle, removing the arresting agent or condition, and then optionally allowing the previously arrested cells to progress through the cell cycle until they reach a predetermined point in the cell cycle. Once the cells have progressed into the desired portion of the cell cycle they can then be treated with a sequence-altering oligonucleotide to give highly efficient oligonucleotide-directed sequence alteration.

In one series of embodiments, the present invention provides methods for increasing the frequency of oligonucleotide-directed sequence alteration by enriching the population of target cells for cells in S phase. The highest frequency of oligonucleotide-directed gene alteration is obtained with cells in S phase. The method comprises synchronizing an otherwise asynchronous population of cells, allowing the synchronized population of cells to proceed into S phase, and performing oligonucleotide-directed sequence alteration on this enriched population.

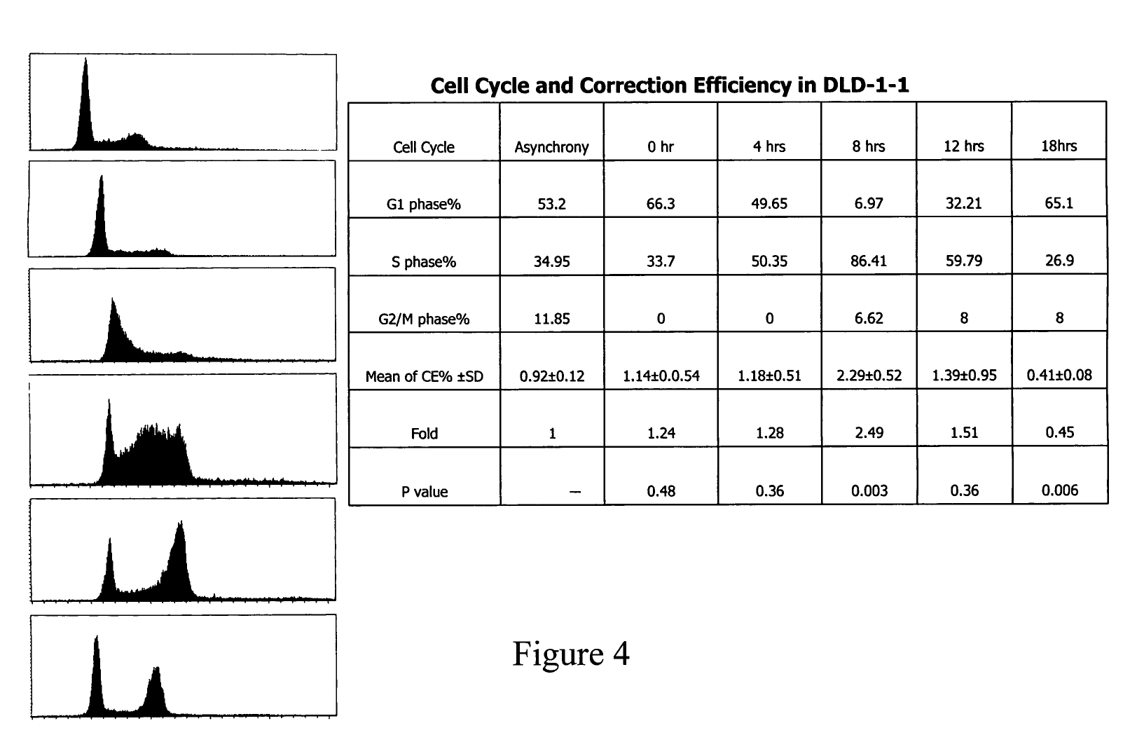

Various means of synchronizing cells may be used in methods of the present invention. DNA replication inhibitors such as mimosine and ciclopirox olamine are known to arrest cells in the cell cycle by inhibiting initiation of DNA replication. Other chemical agents, such as aphidicolin, arrest cells in the cell cycle by inhibiting elongation of DNA replication. In some embodiments of the present invention, cells are grown in media lacking serum (they are “serum starved”) prior to treatment with mimosine. The effects of mimosine treatment on the cell cycle, and on the efficiency of sequence alteration, are illustrated in FIG. 4. Example 2 describes the experimental protocol used to assess the effects of mimosine on sequence alteration. The results show that the highest correction efficiency is observed in populations of cells that are most highly enriched for cells in S phase, with an optimum correction efficiency of 2.49% for a population of cells 86% of which are in S phase.

Conditions such as cold shock can also be used to synchronize populations of cells.

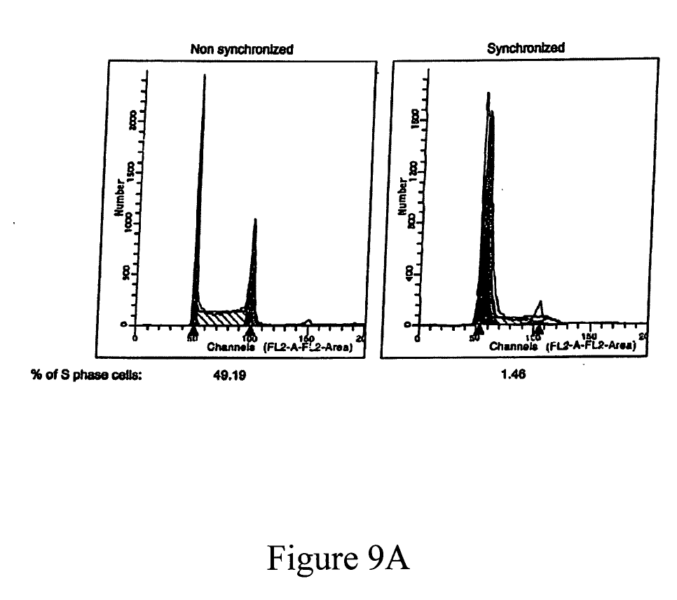

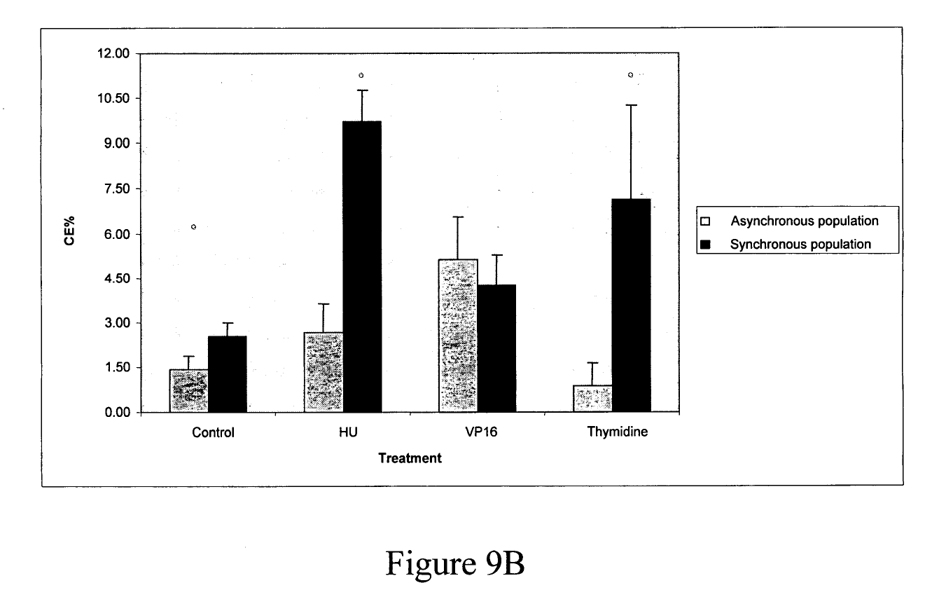

Cells may also be synchronized using double thymidine block (DTB). See, e.g., Lundin et al., J. Mol. Biol. (2003) 328:521-535, the disclosure of which is incorporated herein by reference in its entirety. The effect of a DTB on the cell cycle, and on the efficiency of gene alteration, are illustrated in FIGS. 9A and 9B. Example 4 describes the experimental protocol used to assess the effects of double thymidine block on gene alteration. FIG. 9A shows that DTB decreases the proportion of DLD-1-1 cells in S phase from half to 1.5%, at which time the nearly completely synchronized population of cells is released from growth arrest and allowed to re-enter the cell cycle. After 24 hours of growth the cells are electroporated in the presence of a correcting oligonucleotide. The “Control” data in FIG. 9B show that synchronization by DTB increases correction efficiency.

HU can be used to synchronize growing cells in S phase by blocking or retarding the movement of the replication fork. HU and VP16 also cause stalling of replication forks in mammalian cells in culture, as the cells respond to the DNA damage and the metabolic stress. The use of HU and VP16 to enhance gene alteration is discussed in more detail infra in a section discussing DNA damaging agents.

In methods of performing the function of modulating the cell cycle of cells within a population of target cells according to the present invention, steps for effecting such modulation include, but are not limited to: treating cells with HU, mimosine, VP16, ciclopirox olamine, or aphidicolin; subjecting the cells to double thymidine block; serum starving the cells; or cold shocking the cells. One of skill in the art would recognize that any suitable method of reversibly disrupting the cell cycle of target cells could be used to effect cell cycle synchronization according to the present invention.

Modulating, as used herein, refers to altering the normal progression of the cell cycle in a population of target cells to as to facilitate synchronization of the population of target cells to a given part of the cell cycle.

Techniques for effecting oligonucleotide-directed chromosomal sequence alteration in DLD-1-1 cells are further discussed in Example 1 and in copending U.S. patent application Ser. No. 10/986,418, filed Nov. 10, 2004 (“Mammalian Cell Lines for Detecting, Monitoring, and Optimizing Oligonucleotide-Mediated Chromosomal Sequence Alteration”), the disclosure of which is incorporated herein by reference in its entirety.

The observed enhancement of oligonucleotide-directed gene alteration by cell cycle synchronization, DNA damage and DNA repair may be mechanistically related, for example they may all act by increasing the degree of gene editing taking place at replication forks. Regardless of the mechanism, however, the methods of the present invention dramatically increase the efficiency of gene alteration.

The methods of modulating cell cycle to increase the efficiency of sequence alteration may optionally be combined with other methods to increase efficiency, including other methods disclosed herein.

### DNA Damaging Agents and DNA Repair Induction

Agents that damage DNA, for example by inducing double-stranded breaks (DSBs), can be used to increase the efficiency of gene alteration. These DNA damaging agents may be used alone, or in combination with cell synchronization methods previously described, to obtain enhanced efficiency of sequence alteration. Cell cycle modulating methods and DNA damaging agents may act cumulatively, or in an additive or even in a synergistic way to elevate the frequency of sequence alteration.

In addition to their use as cell cycle arresting agents in synchronizing populations of cells, some DNA replication inhibitors (e.g. HU) may also enhance the efficiency of oligonucleotide-directed gene repair directly by inducing double-stranded breaks in the target DNA and/or by inducing the activity of DNA repair and recombination pathways within the cell.

VP16 (also referred to as etoposide and 4′-demethylepipodophyllotoxin-9-(4,6-O-ethylidene-beta-D-glucopyranoside)) is an anti-cancer drug that also induces DNA double-strand breaks through a specific inhibition of the resealing activity oftopoisomerase II. It is not clear whether VP16-induced breaks occur preferentially at replication forks or at random sites, but both treatments (HU and VP16) have been shown to induce HR pathways, and elevate the frequency of HR, as a result of DNA damage. Besides inducing DNA damage, HU and VP16 also cause stalling of replication forks in mammalian cells in culture, as the cells respond to the DNA damage and the metabolic stress. Chemotherapeutic agents such as VP16 have the advantage that they have been approved for use by the FDA for treatment of patients, and thus may be used for in vivo gene repair, or may be used in ex vivo therapy without the need to thoroughly remove them prior to reintroduction of treated cells into the patient.

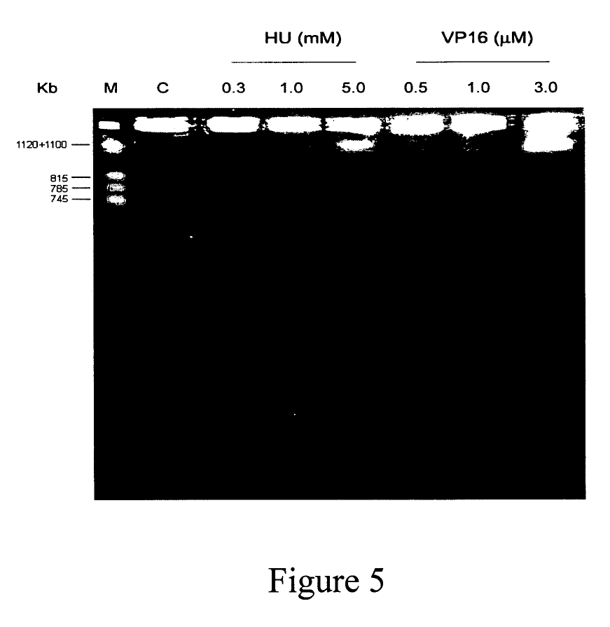

Treatment with HU and VP16 induce DSBs in the DNA of DLD-1-1 cells. FIG. 5 shows a pulsed-field gel of DNA obtained from cells that were untreated (“C”), or treated with various concentrations of HU or VP16, as illustrated. A faint smear of lower molecular weight DNA, the result of DSBs, appears below the high MW bands in the HU and VP16 treated lanes but not in the control.

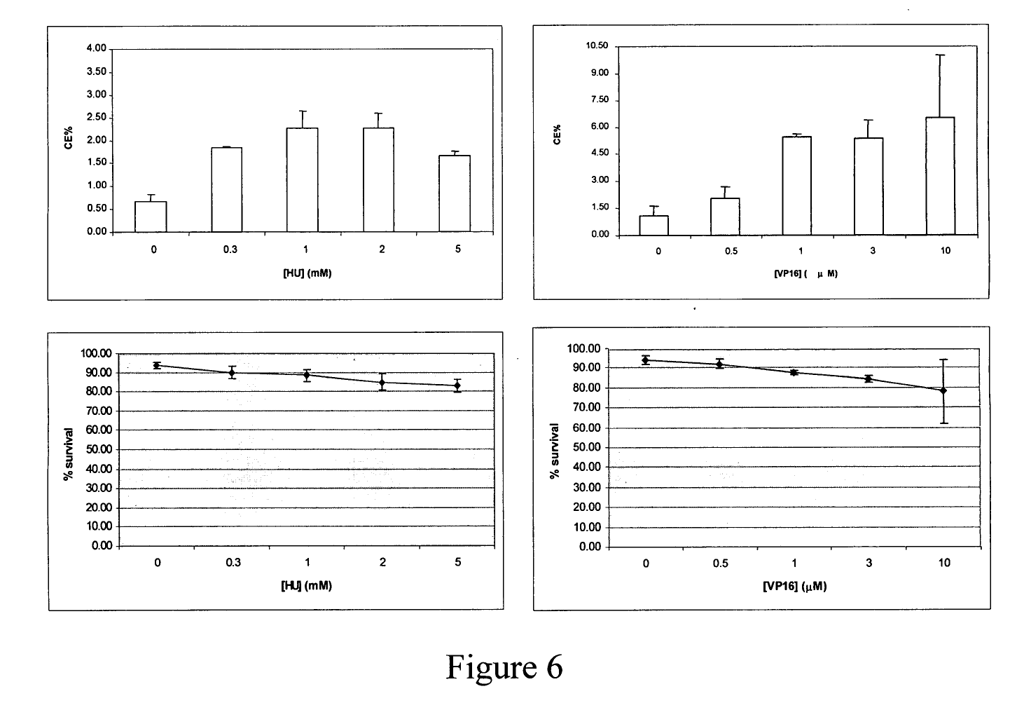

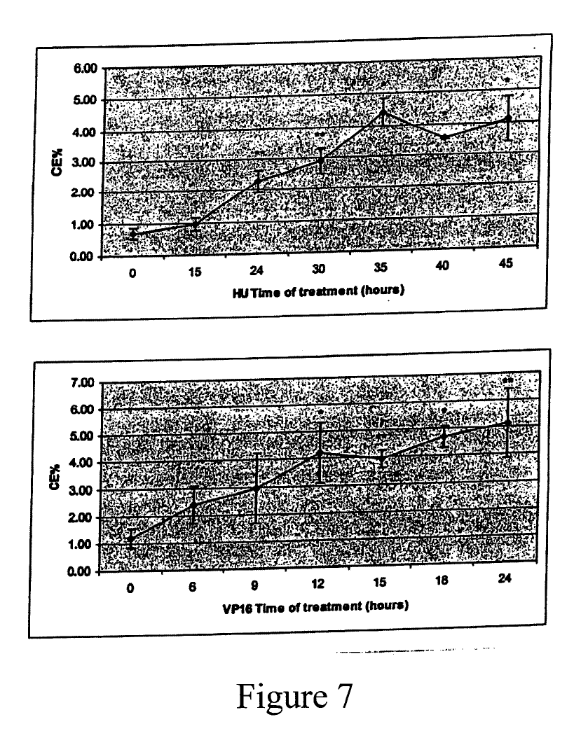

FIG. 6 presents the results of ODSA experiments performed as described in Example 3. DLD-1-1 cells were exposed to various concentrations of HU or VP16 for 24 hours prior to electroporation, washed, and electroporated in the presence of a correcting oligonucleotide (EGFP3 S/72NT). Both HU and VP16 increase correction efficiency in a dose-dependent manner. FIG. 6 also presents survival of cells as a function of treatment with these toxic agents, showing that even at the highest doses approximately 80% or more of cells remain viable. FIG. 7 presents time courses for pretreatment of DLD-1-1 cells with HU and VP16, showing that correction efficiency plateaus at approximately 35 hours for HU and 12-24 hours for VP16.

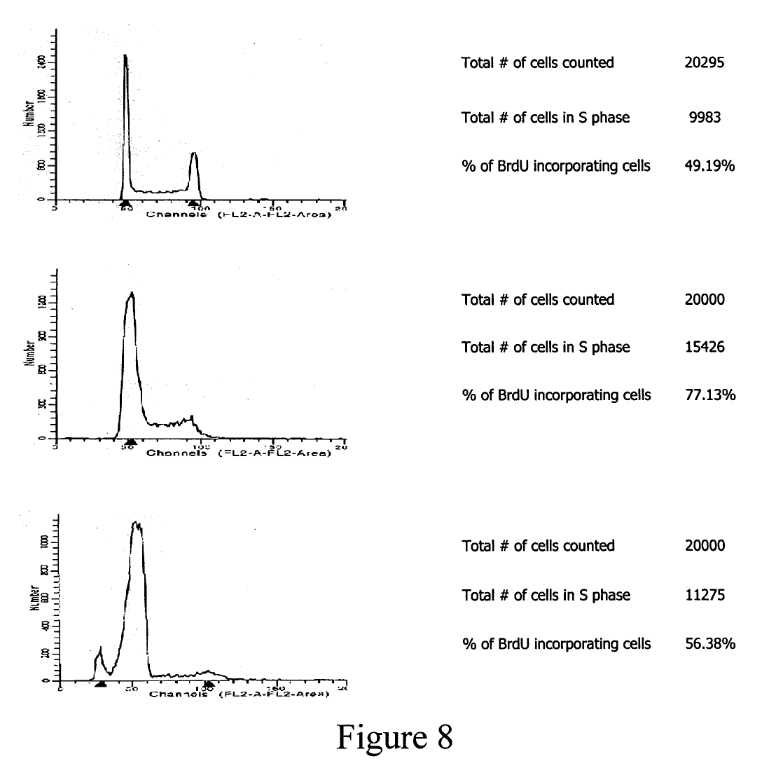

Due to the dual effects of HU and VP16, as both replication inhibitors, and thus cell cycle modulators, and as DNA damaging agents, it is of interest to determine whether the enhancement in correction efficiency shown in FIGS. 6 and 7 is due, at least in part, to the ability of HU and VP16 to modulate the cell cycle. FIG. 8 presents an analysis of the distribution of DLD-1-1 cells in the cell cycle as a function of their treatment with HU or VP16. Cells are treated for 24 hours with nothing, 1 mM HU or 3 μm VP16 prior to FACS analysis. Half of the cells in the untreated culture are in S phase, whereas 56% of VP-16 treated cells, and 77% of HU treated cells, are in S-phase. The results suggest that the effect of HU on correction efficiency may be due at least in part to its effect on the cell cycle.

DNA damaging agents like HU and VP16 can also be used in conjunction with cell synchronization methods to give even greater correction efficiency. For example, oligonucleotide-directed sequence alteration is enhanced in cells that are first synchronized, e.g. by DTB, and then treated with HU. FIG. 9B illustrates the combined effect of cell synchronization by DTB and treatment with DNA damaging agents like HU, VP16 and thymidine, as discussed in more detail at Example 4. FIG. 9A shows that the DTB procedure effectively synchronized the DLD-1-1 cells prior to HU and VP16 treatment, as discussed supra. In the case of HU and thymidine, there is a dramatic increase of correction efficiency when synchronized cells are used as compared to asynchronous cultures. The correction efficiency approaches 10% for HU, more than three times the correction efficiency obtained with asynchronous cells, and 7.5% for thymidine, over seven times the efficiency obtained with asynchronous cells. In contrast, although VP16 gives the highest efficiency in asynchronous cells, the efficiency does not increase when synchronized cells are used.

The results shown in FIG. 9B suggest that HU treatment is acting, at least in part, by some mechanism other than an effect on the cell cycle, such as increasing the number of double strand breaks in the target DNA and/or inducing DNA repair/recombination pathways within the cell. Regardless of the mechanism, treatment of synchronized cells with HU dramatically increases the efficiency of gene repair.

HU may be used at concentrations including 100 mM, 75 mM, 50 mM, 40 mM, 20 mM, 10 mM, 2 mM, 1 mM, 100 μM, 10 μM, 1 μM, 100 nM, 10 nM or lower. The dosage is preferably from about 4 to 100 mM for yeast cells and from about 0.05 mM to 3 mM for mammalian cells. The dosage may be at least 0.05 mM, 0.10 mM, 0.15 mM, 0.20 mM, 0.25 mM, 0.30 mM, 0.35 mM, 0.40 mM, 0.50 mM or more, including at least 0.55 mM, 0.60 mM, 0.65 mM, 0.70 mM, 0.75 mM, 0.80 mM, 0.85 mM, 0.90 mM, 0.95 mM or even 1 mM, 1.1 mM, 1.2 mM, 1.3 mM, 1.4 mM, 1.4 mM, 1.5 mM, 1.6 mM, 1.7 mM, 1.8 mM, 1.9 mM, 2.0 mM, 2.5 mM, 3 mM, or more. Typically, the dosage for mammalian cells is less than about 3.0 mM, and can be less than 2.5 mM, 2.0 mM, 1.5 mM, 1.0 mM, even less than 0.90, 0.85, 0.80, 0.75, 0.70, 0.65, 0.60, 0.55, 0.50, 0.45, 0.40, and even less than about 0.35 or 0.30 mM. Optimal dosing and timing may be determined by routine experimentation, using the assay system set forth in WO 03/075856, the disclosure of which is incorporated herein by reference in its entirety.

In some embodiments DNA damage is induced using alkylating agents (e.g. methyl methanesulfonate (MMS)), antimetabolites (e.g. HU), compounds that form adducts with DNA (e.g. benzopyrene, acetylaminofluorene), topoisomerase II inhibitors (e.g. VP16, VM-26, doxorubicin, 3′-hydroxydaunorubicin, chloroquine, sodium azide, A-74932, clinafloxacin, menogaril, AMSA) or DSB-inducing agents (e.g. bleomycin). In other embodiments of the present invention use DNA damaging agents such as cis-platin, sodium arsenite, restriction endonucleases, sodium vanadate, ethidium bromide (EtBr), chloroquine, VP26, and heavy metal ions such as cadmium and zinc. Note that the categories of agents listed herein are not necessarily mutually exclusive, i.e. it is possible for chemical agents useful in the methods of the present invention to fall into more than one of the aforementioned categories (e.g. a single agent may be categorized as a topoisomerase II inhibitor and a DNA damaging agent).

In yet other embodiments of the present invention, DNA damage is effected by physical means, such as exposure to ultraviolet light or other ionizing radiation. Exposure to ultraviolet light may be mimicked by exposure to 4-nitroquinoline-N-oxide (4NQO). Means to accomplish the function of inducing DNA damage include all treatments and agents listed here and their equivalents. One of skill in the art would recognize that any suitable DNA damaging agents or treatment could be used in place of those listed here and still fall within the scope of the present invention.

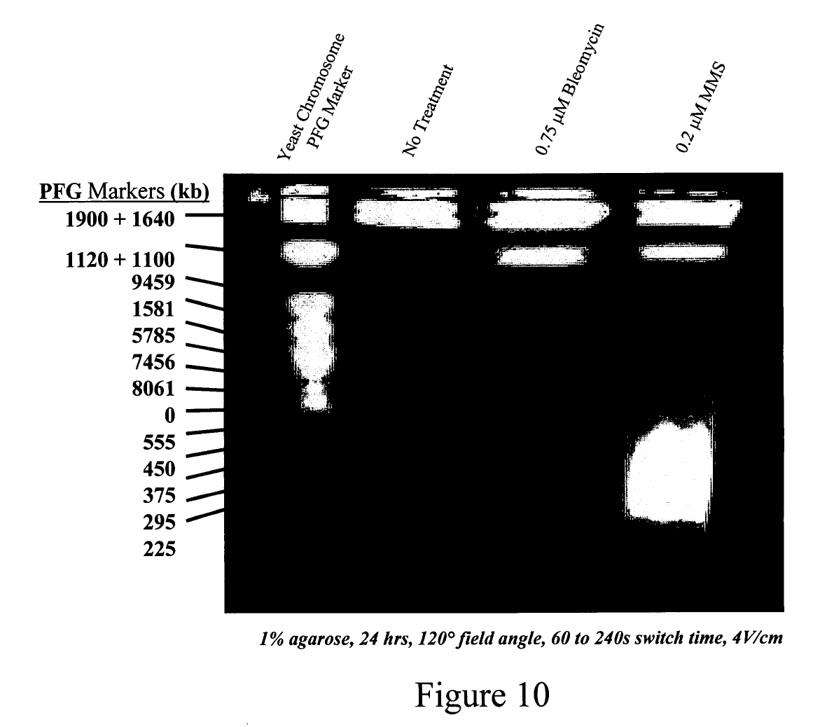

FIG. 10 presents a pulsed-field gel showing DNA damage caused by treatment of DLD-1-1 cells with 0.75 μM bleomycin and 0.2 μM MMS, as described in more detail at Example 5. The lower bands in the bleomycin and MMS lanes, and the low smear in the MMS lane, which are not present in the “no treatment” control, represent DNA fragments that are the result of DSBs induced by these agents. The data confirm that these agents damage DNA in DLD-1-1 cells under the experimental conditions used.

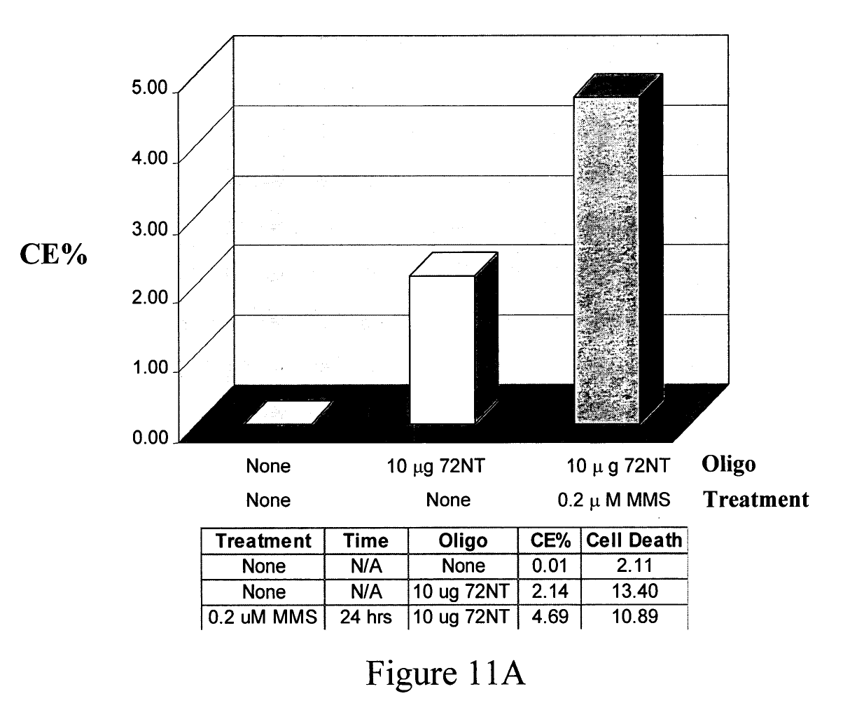

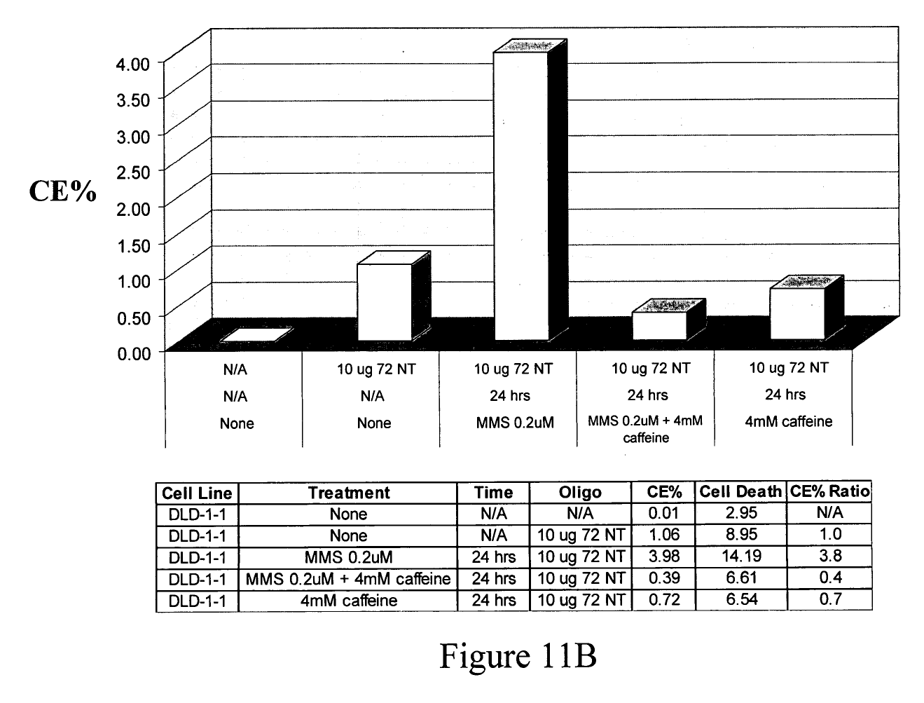

FIG. 11A presents the results of gene correction experiments performed on MMS-treated DLD-1-1 cells, showing that MMS pre-treatment more than doubles the correction efficiency. FIG. 11B presents replicates of the experiments illustrated in FIG. 11A, and also includes experiments in which cells are treated with 4 mM caffeine, with or without 0.2 μM MMS. Both caffeine experiments gave correction efficiencies lower than the control experiment with no treatment other than the correcting oligonucleotide.

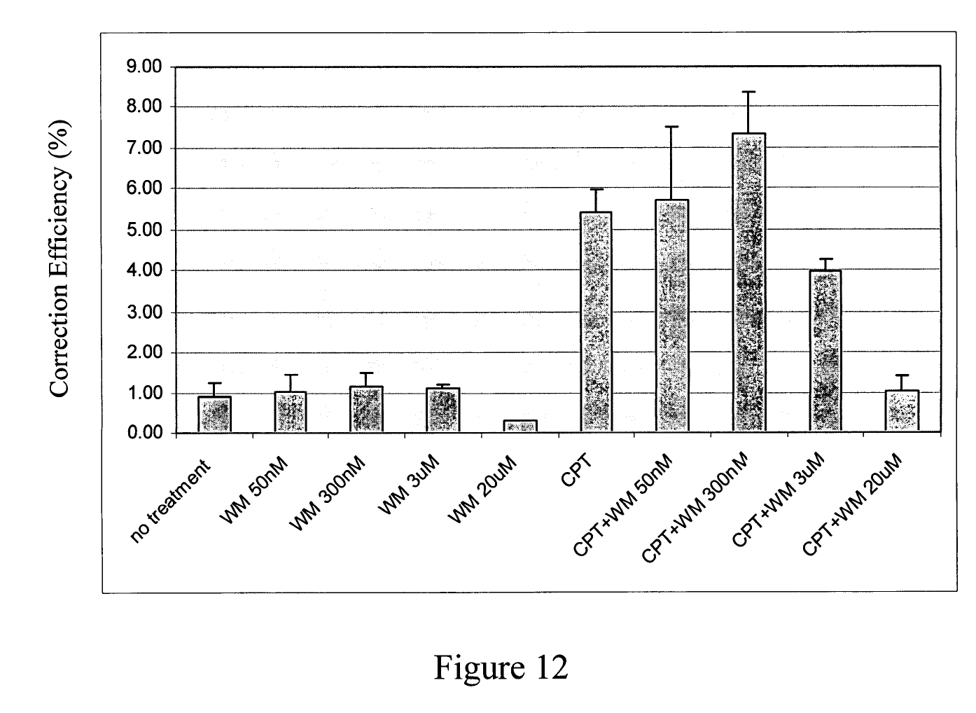

FIG. 12 presents the results of experiments to test the effect of pretreatment of cells with wortmannin (WM). DLD-1-1 cells are treated with WM at the concentrations indicated, with or without 30 nM CPT, for 24 hours prior to electroporation in the presence of a correcting oligonucleotide.

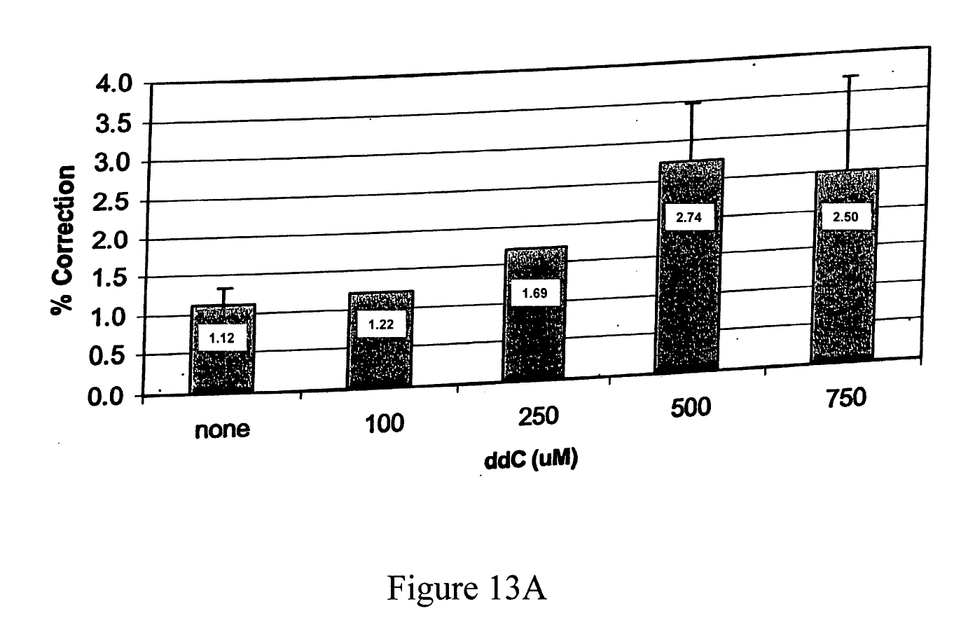

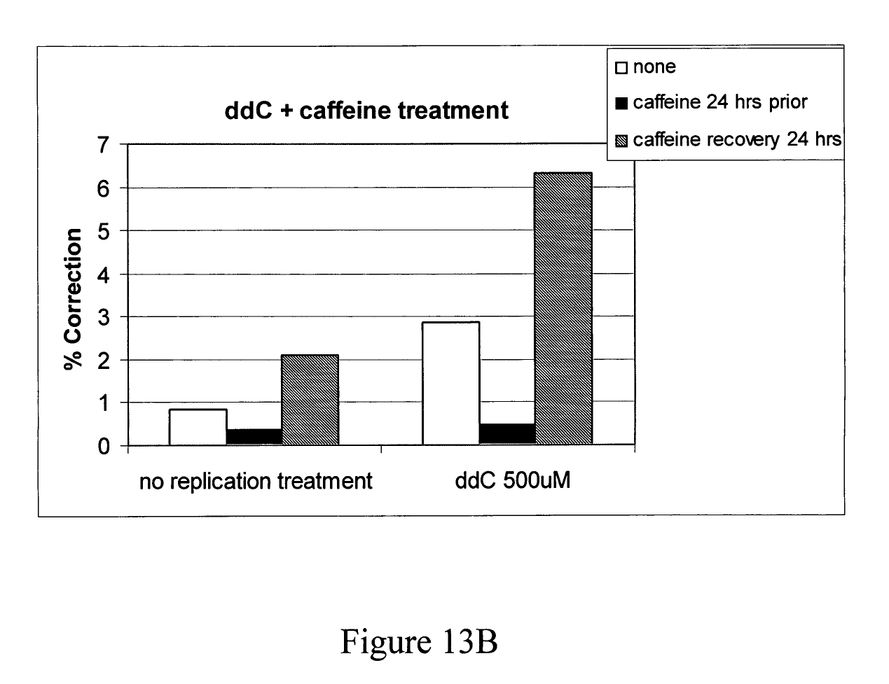

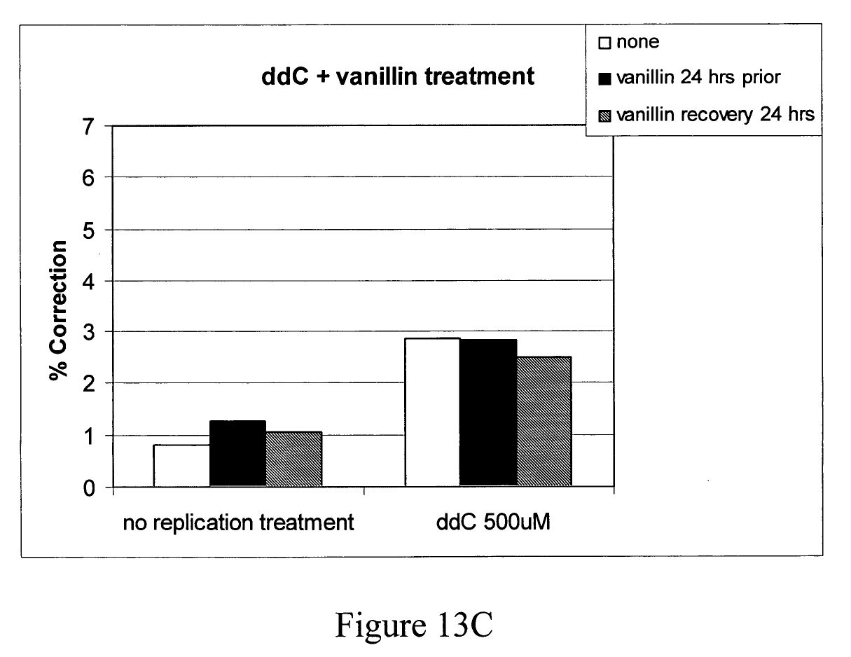

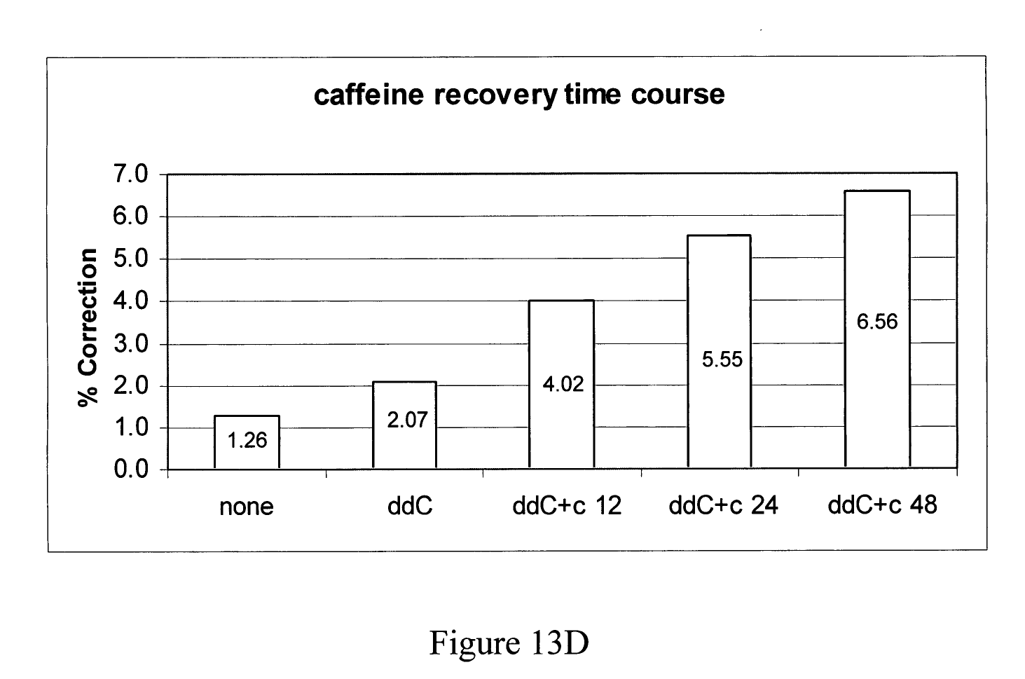

FIG. 13A presents the results of experiments to test the effect of pretreatment of cells with dideoxycytidine (ddC) on correction efficiency in the DLD-1-1 system. ddC increases correction efficiency more than two-fold when used at 500-750 μM. FIG. 13B presents results of experiments in which caffeine was used to treat DLD-1-1 cells either before electroporation (“prior”), or after electroporation (“recovery”), with or without ddC treatment. The results show that although caffeine decreases correction efficiency when used as a pretreatment, as observed when caffeine is used in conjunction with MMS (FIG. 11B), it dramatically increases correction efficiency when used in the recovery phase. FIG. 13D shows that the effect of caffeine improves the longer it is included in the recovery phase up to the longest time tested (48 hours). FIG. 13C demonstrates that Im M vanillin has no effect on correction efficiency regardless of when it is added, and whether or not it is combined with ddC treatment.

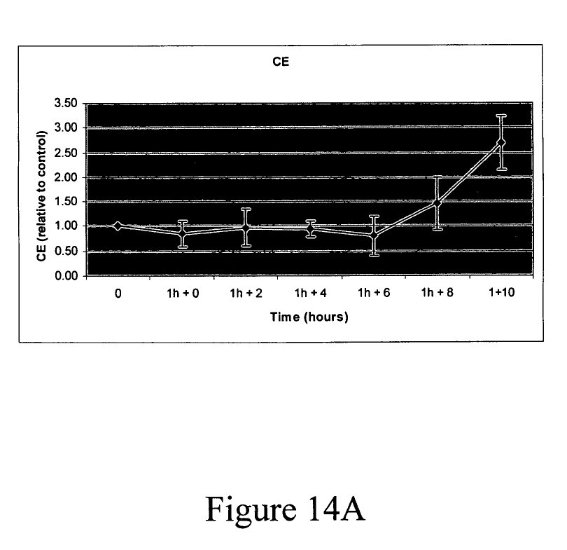

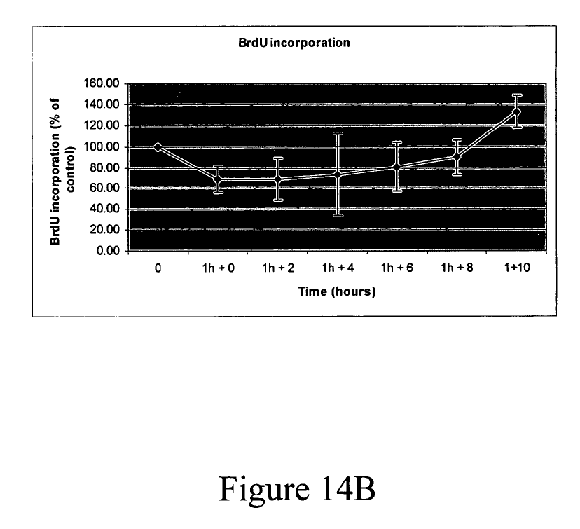

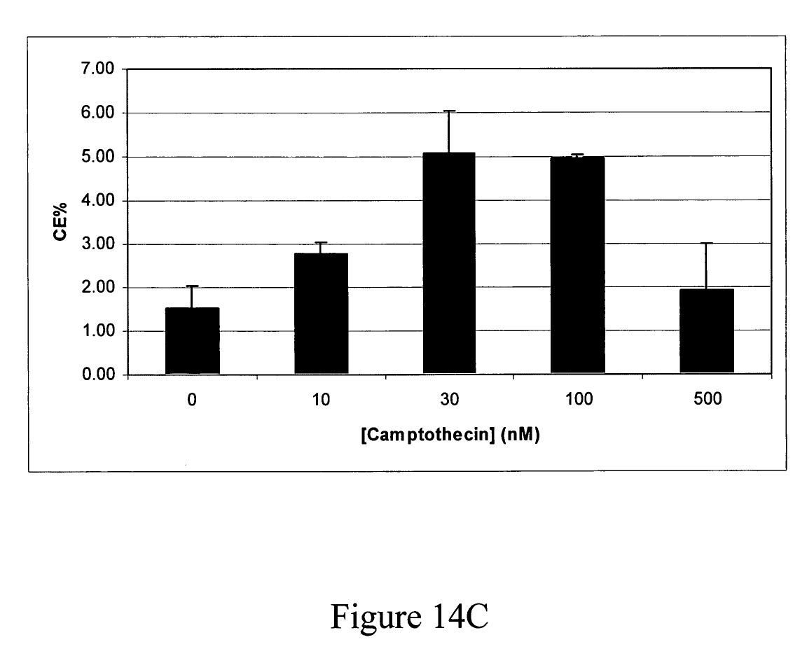

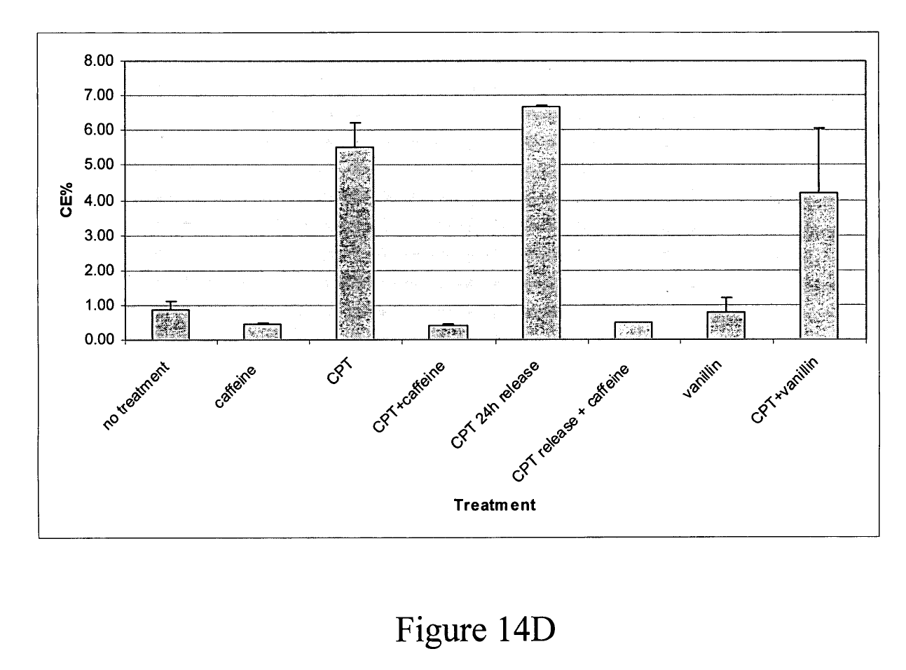

FIGS. 14A and 14B show the results of treatment of cells with 3 μM CPT for one hour, followed by release for various times (0-10 hours). For example, “1H+10” data points refer to cells treated with CPT for one hour and then incubated in fresh CPT-free medium for 10 hours prior to BrdU labeling or electroporation. Data points marked “0” time are cells not treated with CPT.

FIG. 14A presents BrdU incorporation (as a percentage of control) for DLD-1-1 cells that are either untreated, or treated with 3 μM camptothecin (CPT) for one hour, at which point CPT is washed out and fresh CPT-free medium is added. Cell are then incubated for various times prior to BrdU labeling, and BrdU incorporation is plotted as a function of post-CPT incubation time. FIG. 14B presents correction efficiency (relative to control) for DLD-1-1 cells treated in the same way as those in FIG. 14A, except that the treated cells are electroporated in the presence of a correcting oligonucleotide rather than BrdU labeled.

FIG. 14C shows that pretreatment of DLD-1-1 cells with CPT triples correction efficiency when used at 30-100 nM.

FIG. 14D presents correction efficiency (as a percentage) in a series of ODSA experiments as a function of treatment with CPT alone or in combination with other agents and related controls. DLD-1-1 cells are treated with the agents shown for one hour, either concurrently or sequentially, as indicated. The treated cells are then electroporated in the presence of a correcting oligonucleotide and correction efficiency is determined. From left to right, cells are untreated, or treated with 4 mM caffeine, 30 nM CPT, or a mixture of 4 mM caffeine and 30 nM CPT. “CPT 24 h release” refers to cells that are treated with 30 nM CPT, followed by a wash step and incubation in fresh medium for another hour prior to electroporation. The next data point is similar but includes 4 mM caffeine in the second one hour incubation. Data are also presented for treatment with 1 mM vanillin and a mixture of 1 mM vanillin and 30 nM CPT.

Benefits of Highly Efficient Oligonucleotide-Directed Gene Alteration

Embodiments of the present invention may be useful in conducting gene therapy in plants or animals, including humans. Ex vivo gene therapy involves the removal of cells from an organism, in vitro gene therapy, and replacement of the treated cells into the host organism (or, in some embodiments, a different host organism). For example, many human diseases may be treated by effecting changes in the chromosomes of hematopoietic stem cells by removing such cells from a sample of peripheral blood, effecting a genetic alteration, and reintroducing the treated cells into the patient's bloodstream. ODSA methods of the present invention, involving cell cycle modulation to increase the efficiency of gene alteration, can be used to effect gene repair in these isolated hematopoietic stem cells. In some embodiments, cells undergoing ex vivo gene therapy are treated using methods designed to protect them from damage, such as the method described in U.S. patent application publication no. US 2003/0134789 A1, the disclosure of which is incorporated herein by reference in its entirety.

The methods and kits of the present invention may be used with any oligonucleotide that directs targeted alteration of nucleic acid sequence. For example, oligonucleotides may be designed to alter sequences in many human genes including, e.g., ADA, p53, beta-globin, RB, BRCA1, BRCA2, CFTR, CDKN2A, APC, Factor V, Factor VIII, Factor IX, hemoglobin alpha 1, hemoglobin alpha 2, MLH1, MSH2, MSH6, ApoE, LDL receptor, UGT1, APP, PSEN1, and PSEN2. Additional genes are listed infra.

The methods and kits of the invention typically increase the efficiency of gene alteration using oligonucleotide-directed nucleic acid sequence alteration by at least about two-fold relative to the efficiency obtained using a population of targeted cells that has not previously been treated according to a method of the invention. The increased efficiency of gene alteration can be at least about two, three, four, five, six, seven, eight, nine, ten, twelve, fifteen, twenty, thirty, and fifty or more fold.

The methods and kits of the invention may also increase the efficiency of gene alteration using oligonucleotide-directed nucleic acid sequence alteration to correction efficiencies of at least about 0.2, 0.4, 0.6, 0.8, 1, 1.2, 1.2, 1.4, 1.6, 1.8, 2, 2.2, 2.4, 2.6, 2.8, 3, 3.3, 3.7, 4, 4.5, 5, 5.5, 6, 6.5, 7, 7.5, 8, 8.5, 9, 9.5, 10, 11, 12, 13, 14, 15, 17 percent or more.

In some embodiments, efficiency of conversion is defined as the percentage of recovered substrate target molecules that have undergone a conversion event. Alternatively, depending on the nature of the target genetic material (e.g. an extrachromosomal element in a cell), efficiency could be represented as the proportion of cells or clones containing an extrachromosomal element that exhibits a particular phenotype. In embodiments in which the sequence-altering oligonucleotide directs an alteration that produces a selectable phenotype, the efficiency of conversion can be expressed as the proportion of targeted cells (or clones thereof) that exhibit the selectable phenotype as a fraction of the total number of targeted cells (or clones thereof) assayed for the selectable phenotype. Alternatively, for embodiments in which the phenotype conferred by the alteration is a non-selectable, representative samples of the target genetic material can be analyzed, e.g. by sequencing, allele-specific PCR or comparable techniques, to determine the percentage that have acquired the desired change.

### Compatible Cell Types

Alterations may usefully be made according to the methods of the present invention in mammalian cells, including human cells, such as liver, lung, colon, cervix, kidney, and epithelium cells.

Cultured mammalian cells that usefully may be targeted for desired sequence alteration according to the methods of the present invention include HTT1080 cells (human epithelial fibrosarcoma), COS-1 and COS-7 cells (African green monkey), CHO-K1 cells (Chinese hamster ovary), H1299 cells (human epithelial carcinoma, non-small cell lung cancer), C1271 (immortal murine mammary epithelial cells), MEF (mouse embryonic fibroblasts), HEC-1-A (human uterine carcinoma), HCT15 (human colon cancer), HCT116 (human colon carcinoma), LoVo (human colon adenocarcinoma), and HeLa (human cervical carcinoma) cancer cells as well as PC12 cells (rat pheochromocytoma).

Alterations in cultured mammalian cells may usefully be made to create coisogenic cell collections, as described in copending international patent application published as WO 03/027264 and U.S. patent application Ser. No. 10/260,638, the disclosures of which are incorporated herein by reference in their entireties. Genes usefully targeted in such coisogenic collections include loci affecting drug resistance (equivalently, drug sensitivity) or drug metabolism, including: CYP1A2, CYP2C17, CYP2D6, CYP2E, CYP3A4, CYP4A11, CYP1B1, CYP1A1, CYP2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6D1, CYP6F1, CYP7A1, CYP8, CYP11A, CYP11B1, CYP11B2, CYP17, CYP19, CYP21A2, CYP24, CYP27A1, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)-alpha, glutathione S-transferase-mu, glutathione S-transferase-pi, ACE, and KCHN2.

In other embodiments, cells within which targeted alterations may usefully be effected according to the methods of the present invention include progenitor and stem cells—both embryonic (ES) stem cells and non-ES cells such as hematopoietic progenitor or stem cells, including CD34⁺CD38⁻ hematopoietic progenitor and stem cells and muscle-derived stem cells.

In certain ex vivo embodiments of the methods of the present invention, in which targeted sequence alterations are made in human non-ES cells, such as hematopoietic progenitor or stem cells, such as CD34⁺CD38⁻ hematopoietic stem cells, the sequence-altered cells can be reintroduced into a human subject for ex vivo gene therapies.

ES cells can be mammalian ES cells, either non-human mammalian ES cells or human ES cells; human ES cells may, e.g., be from a cell line approved for use in the jurisdiction in which the methods, compositions and kits of the present invention are to be used. For example, for use in the United States, any human stem cell line that does not violate state or federal law may be used, such as those cell lines that meet United States federal funding criteria; the National Institutes of Health maintains a list of these existing stem cell lines (http://escr.nih.gov) that includes those held by the following: BresaGen, Inc., Athens, Ga. (2 available lines); ES Cell International, Melbourne, Australia (6 available lines); MizMedi Hospital—Seoul National University, Seoul, Korea (1 available line); Technion-Israel Institute of Technology, Haifa, Israel (2 available lines); University of California, San Francisco, Calif. (1 available line); Wisconsin Alumni Research Foundation, Madison, Wis. (5 available lines).

In some ex vivo embodiments of the methods of the present invention, the targeted sequence alterations are made in human ES cells, which are thereafter used, where legally permissible, to generate tissue or, where permitted, a viable embryo.

### Non-Human Mammalian Cells

In certain ex vivo embodiments of the methods of the present invention, in which targeted sequence alterations are made in non-human cells, such as non-human mammalian ES cells or plant cells, the sequence-altered cells can be used to generate intact organisms, which can thereafter be propagated.

For example, the methods of the present invention can be used to create genetically altered animals, including livestock—such as cattle, bison, horses, goats, sheep, pigs, chickens, geese, ducks, turkeys, pheasant, ostrich and pigeon—to enhance expression of desirable traits, and/or decrease expression of undesirable traits, by first creating genetically altered cells. In other embodiments, the methods of the present invention can be used to create genetically altered animals useful as laboratory models, such as rodents, including mice, rats, guinea pigs; lagomorphs, such as rabbits; monkeys; apes; dogs; and cats. Methods for producing transgenic animals comprising genetically modified cells are known in the art, and are disclosed, for example, in WO 00/51424, “Genetic Modification of Somatic Cells and Uses Thereof,” the disclosure of which is incorporated herein by reference in its entirety.

Further aspects of the present invention are the non-human animals produced thereby.

### Plant Cells

In yet other embodiments, the cells within which targeted alterations are made are plant cells.

Desirable phenotypes that may be obtained in plants by known nucleic acid sequence alterations include, for example, herbicide resistance; male- or female-sterility; salt, drought, lead, freezing and other stress tolerances; altered amino acid content; altered levels or composition of starch; altered levels or composition of oils; and elimination of epitopes in gluten that are known to instigate autoimmune responses in individuals with celiac disease.

Particularly useful plants from which the cells to be used may be drawn include, for example, experimental model plants such as Chlamydomonas reinhardtii, Physcomitrella patens, and Arabidopsis thaliana in addition to crop plants such as cauliflower (Brassica oleracea), artichoke (Cynara scolymus), fruits such as apples (Malus, e.g. domesticus), mangoes (Mangifera, e.g. indica), banana (Musa, e.g. acuminata), berries (such as currant, Ribes, e.g. rubrum), kiwifruit (Actinidia, e.g. chinensis), grapes (Vitis, e.g. vinifera), bell peppers (Capsicum, e.g. annuum), cherries (such as the sweet cherry, Prunus, e.g. avium), cucumber (Cucumis, e.g. sativus), melons (Cucumis, e.g. melo), nuts (such as walnut, Juglans, e.g. regia; peanut, Arachis hypogeae), orange (Citrus, e.g. maxima), peach (Prunus, e.g. persica), pear (Pyra, e.g. communis), plum (Prunus, e.g. domestica), strawberry (Fragaria, e.g. moschata or vesca), tomato (Lycopersicon, e.g. esculentum); leaves and forage, such as alfalfa (Medicago, e.g. sativa or truncatula), cabbage (e.g. Brassica oleracea), endive (Cichoreum, e.g. endivia), leek (Allium, e.g. porrum), lettuce (Lactuca, e.g. sativa), spinach (Spinacia, e.g. oleraceae), tobacco (Nicotiana, e.g. tabacum); roots, such as arrowroot (Maranta, e.g. arundinacea), beet (Beta, e.g. vulgaris), carrot (Daucus, e.g. carota), cassaya (Manihot, e.g. esculenta), turnip (Brassica, e.g. rapa), radish (Raphanus, e.g. sativus), yam (Dioscorea, e.g. esculenta), sweet potato (Ipomoea batatas); seeds, including oilseeds, such as beans (Phaseolus, e.g. vulgaris), pea (Pisum, e.g. sativum), soybean (Glycine, e.g. max), cowpea (Vigna unguiculata), mothbean (Vigna aconitifolia), wheat (Triticum, e.g. aestivum), sorghum (Sorghum e.g. bicolor), barley (Hordeum, e.g. vulgare), corn (Zea, e.g. mays), rice (Oryza, e.g. sativa), rapeseed (Brassica napus), millet (Panicum sp.), sunflower (Helianthus annuus), oats (Avena sativa), chickpea (Cicer, e.g. arietinum); tubers, such as kohlrabi (Brassica, e.g. oleraceae), potato (Solanum, e.g. tuberosum) and the like; fiber and wood plants, such as flax (Linum e.g. usitatissimum), cotton (Gossypium e.g. hirsutum), pine (Pinus sp.), oak (Quercus sp.), eucalyptus (Eucalyptus sp.), and the like and ornamental plants such as turfgrass (Lolium, e.g. rigidum), petunia (Petunia, e.g. x hybrida), hyacinth (Hyacinthus orientalis), carnation (Dianthus e.g. caryophyllus), delphinium (Delphinium, e.g. ajacis), Job's tears (Coix lacryma-jobi), snapdragon (Antirrhinum majus), poppy (Papaver, e.g. nudicaule), lilac (Syringa, e.g. vulgaris), hydrangea (Hydrangea e.g. macrophylla), roses (including Gallicas, Albas, Damasks, Damask Perpetuals, Centifolias, Chinas, Teas and Hybrid Teas) and ornamental goldenrods (e.g. Solidago spp.).

Generally, the oligonucleotides are administered to isolated plant cells or protoplasts according to a method of the present invention and the resulting cells are used to regenerate whole plants according to any method known in the art.

The cells within which targeted alterations are effected according to the methods of the present invention can be primary isolated cells, selectively enriched cells, cultured cells, or tissue explants.

### Candidate Genes for In/Ex Vivo Gene Therapy

In vivo gene repair according to the present invention may be used to alter genes that are associated with various human diseases. Alternatively, ex vivo methods can be used to alter genes in cells that have been removed from an organism (e.g. the patient) in vitro, so that they may be subsequently introduced (or reintroduced) into a patient. Various known mutations of specific genes are known to cause disease, and thus it is relatively straightforward to design oligonucleotides to repair the mutations. Genes known to cause human disease include, but are not limited to, p53, BRCA1, BRCA2, CDKN2A, APC, RB, MLH1, MSH2, MSH6, AD1, AD2, AD3, AD4, and the gene for clotting factor V. Such embodiments are further discussed in copending U.S. patent application Ser. No. 10/681,074, filed Oct. 7, 2003 (“Methods and Compositions for Reducing Screening in Oligonucleotide-Directed Nucleic Acid Sequence Alteration”), the disclosure of which is incorporated herein by reference in its entirety.

Other diseases (and their corresponding genes) that may be amenable to treatment by methods of the present invention include Alpha-Thalassemia (α-Globin HBA1, HBA2), Sickle Cell Disease (β-Globin (HBB)), Beta-Thalassemia (B-Globin (HBB)), Hemophilia A (FVIII), Hemophilia B (Christmas disease) (FIX), Von Willebrand Disease (VWF), McLeod syndrome (MLS) (XK), Hereditary Spherocytosis (ANK1, SBTB, SLC4A1), Elliptocytosis/Poikilcytosis (SPTA1, EBP41), RBC Pyruvate Kinase Deficiency (PK-LR), G-6-P dehydrogenase deficiency (G6PDH), Huntington Chorea (HD (HTT), JPH3), Alzheimer's (APP1, APOE, PSEN1, PSEN2, PLCD1), Amyotrophic Lateral Sclerosis (SOD1), Rett Syndrome (MECP2), Fragile X (FMR1), Spinal muscular atrophy (SMA) (SMN1, SMN2), Gaucher (glucocerebrosidase), Pompe (a-1,4-glucosidase or acid maltase deficiency (GAA)), Fabry (alpha-Gal A (GLA)), Krabbe (Galactosylceramidase gene (GALC)), Tay-Sachs (Hexosamidase A (HEXA)), Sandhoff Disease (Hexosamidase B (HEXB)), Niemann Pick (Sphinogmyelinase A and B (NPC1, NPC2)), Mucolipidosis 11 (1-cell disease) (a-L-Iduronidate sulfatase (GNPTA)), Mucolipidosis III (m13c), Mucolipidosis IV (MCOLN1), MPS-I, Hurler (a-1-L-iduronidase (IUAD)), MPS-I Scheie's Disease (a-1-L-iduronidase (IUAD)), MPS II, Hunter (IDURONATE 2-SULFATASE (IDS), MPSIIIA, Sanfilippo Syndrome A (SGSH), MPSIIIB, Sanfilippo Syndrome B (Alpha-N-acetylglucosaminidase deficiency(NAGLU)), Aldosterone Deficiency (CYP11B2), Bardet-Biedi Syndrome (BBS1), Byler Syndrome (ATP8B1), Congenital Nephotic Syndrome (NPHS1), Glutaric Acidurea, Type I (GCDH), Glycogen Storage Disease, Type 6 (PYGL), Hirschsprung (EDNRB), Maple syrup urine disease (BCKDHA, BCKDHB, DBT), Medium chain acyl-CoA dehydrogenase deficiency (ACADM), Mevalonate kinase deficiency (MVK), Microcephaly with 2-ketoglutaric aciduria (SLC25A19), Propionic acidemia (PCCA, PCCB), 3-B-hydroxysteroid dehydrogenase deficiency (HSD3B2), 3-methylcrotonylglycinuria (MCCC2), Homocystinuria (MTHFR), Cystinurea (SL3A1, SLC7A9), Cystinosis (Cystinosin (CTNS)), Polycystic Kidney Disease, Dominant (PKD1, PKD2), Polycystic Kidney Disease, Recessive (PKHD1), Wolman Sydrome (Sphingolipidoses-acid lipase), Farber's Disease (Ceramidase (ASAH)), Austins' disease (Multiple sulfatases (ARSA)), MPS VII (GUSB), Canavan Disease (ASPA), Phenylketonuria (PAH), Criggler-Najjar Type I (UDPGT), Criggler-Najjar Type II (UGTIA1), Gilbert's Syndrome (UGT1A1), Lesch-Nyhan (HPRT), Ornithine Transcarbamylase Deficiency (OTC), Hereditary Hemochromatosis (HFE, TFR), Tyrosinemia Type 1 (HT1) (FAH, MOD2), Tyrosinemia, type 3 (HPD), Porpheria (FC), Diabetes (GCK), Antitrypsin alpha 1 deficiency (AAT), ADA Deficiency (ADA), SCID (DNA-PK, RAG1, RAG2), XLAAD (Foxp3), XSCID (IL2RG), Chronic Granulomatous Disease (CYBA, CYBB, NCF1, NCF2), Nemaline rod myopathy (TNNT1), Familial Periodic Fever (TRAPS) (TNFRSF1A), Duchennes Muscular dystrophy (DMD), Cystic Fibrosis (CFTR), Epidermolysis Bullosa (Col7A1, Col 7A-1, LAMA3, LAMB3, LAMB4, LAMC2), Gyrate atrophy (OAT), Marfan Syndrome (FBN1), Alport Syndrome (COL4A3, COL4A4, Col4A5), Cartilage Hair Hyplasia (RMRP), Ellis-Van Creveld (EVC), McKusick-Kauffinan syndrome (MKKS), Osteogenesis imperfecta (Col1A2), Hypercholesterolemia (LDLR), Familial Hypercholanemia (BAAT, TJP2), Hyperlipidemia (APOE), Thrombosis (AT), spinal muscular atrophy (SMN1, SMN2), and Sitosterolemia (ABCG8, ABCG5). Diseases such as Crigler-Najjar and CAII deficiency are also candidates for gene therapy using the methods of the present invention. Genes that are amenable as targets for the methods of the present invention are also disclosed in Liu et al., Nat. Rev. Genetics (2003) 4(9):679-89 and Anderson et al., J. Mol. Med. (2002) 80:770-781, the disclosures of which are incorporated herein by reference in their entireties.

Certain human diseases are particularly amenable to ex vivo gene therapy, which in one variation involves gene therapy performed on cells isolated from a patient and subsequently re-introduced to the patient after treatment. Candidate diseases for ex vivo gene therapy include, but are not limited to, neurodegenerative diseases, bone regenerative disorders, diabetes, Alzheimer's disease, Parkinson's disease, familial hypercholesterolemia, inherited hyperbilirubinemias, osteoarthritis (OA), junctional epidermolysis bullosa (JEB), metastatic renal-cell carcinoma (RCC), prostate cancer and lysosomal storage disorders such as Fabry's, Gaucher's, Pompe's and Niemann-Pick diseases. Gene therapy may be performed on extracted blood or bone marrow cells that can be reintroduced to the patient with greatly decreased risk of adverse reaction. Cell types that are promising targets for ex vivo gene therapy include bone marrow stem cells, liver cells, blood vessel smooth muscle cells and tumor-infiltrating lymphocytes (for cancer treatment).

The methods of the present invention are well suited to such ex vivo methods since they involve treatment of the patient's cells or tissues with agents that do not persist after gene therapy. The methods also increase the efficiency of ODSA to levels that may give rise to therapeutic effects when treated cells are reintroduced into the patient, as opposed to prior methods that effect alteration in too few cells to have any clinical effect.

### Fabry's Disease

Fabry's disease is an X-linked recessive lysosomal storage disorder caused by a deficiency of lysosomal α-galactosidase A, encoded by the GLA gene. Brady and Schiffman, JAMA (2001) 285(2):169. Several allelic variants of the 12 kb long GLA gene are associated with disease phenotypes. Patients homozygous for deleterious mutations in GLA can suffer severe painful neuropathy with progressive renal, cardiovascular and cerebrovascular dysfunction and early death. Id

Further information on Fabry's disease (and other human diseases discussed herein), and their related genetic mutations, is available through the Online Mendelian Inheritance in Man (OMIM) database, accessible via the Entrez Pubmed website at <http://www.ncbi.nlm.nih.gov/entrez>. The MIM code for Fabry's disease is MIM+301500.

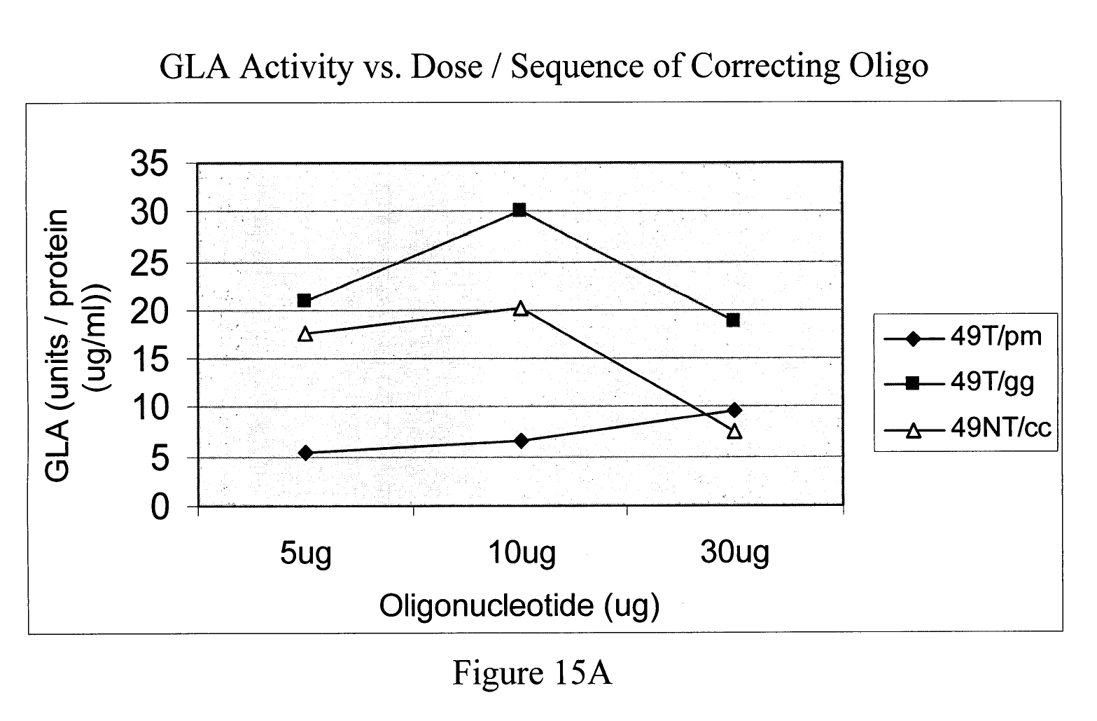

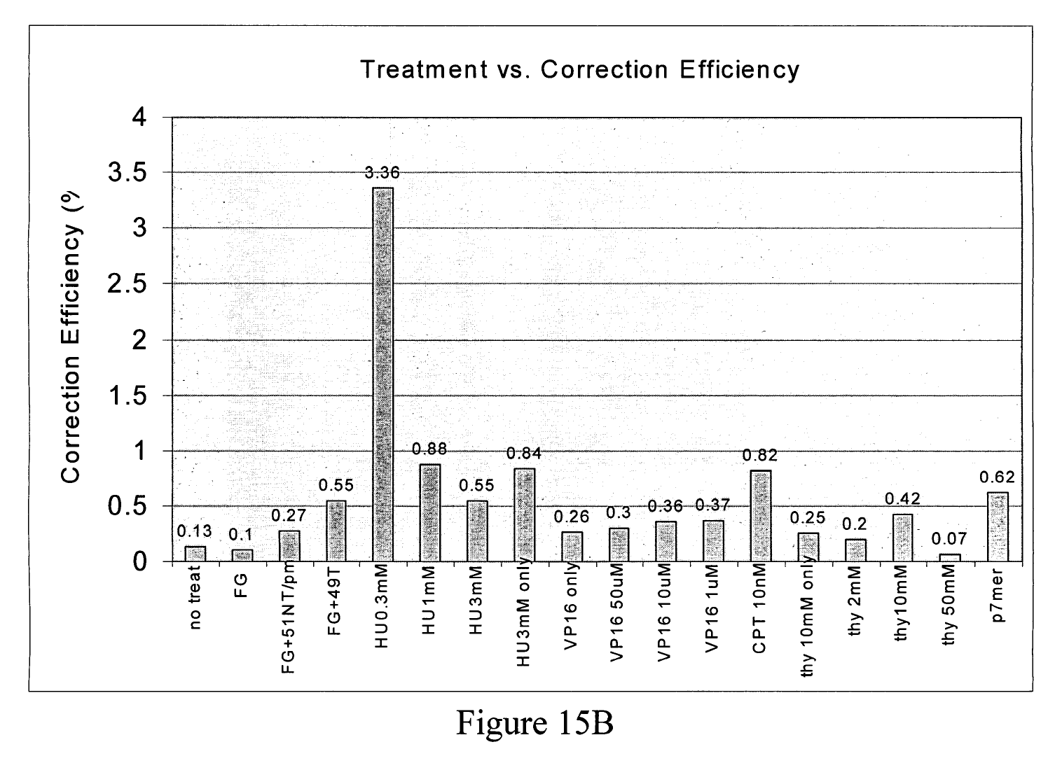

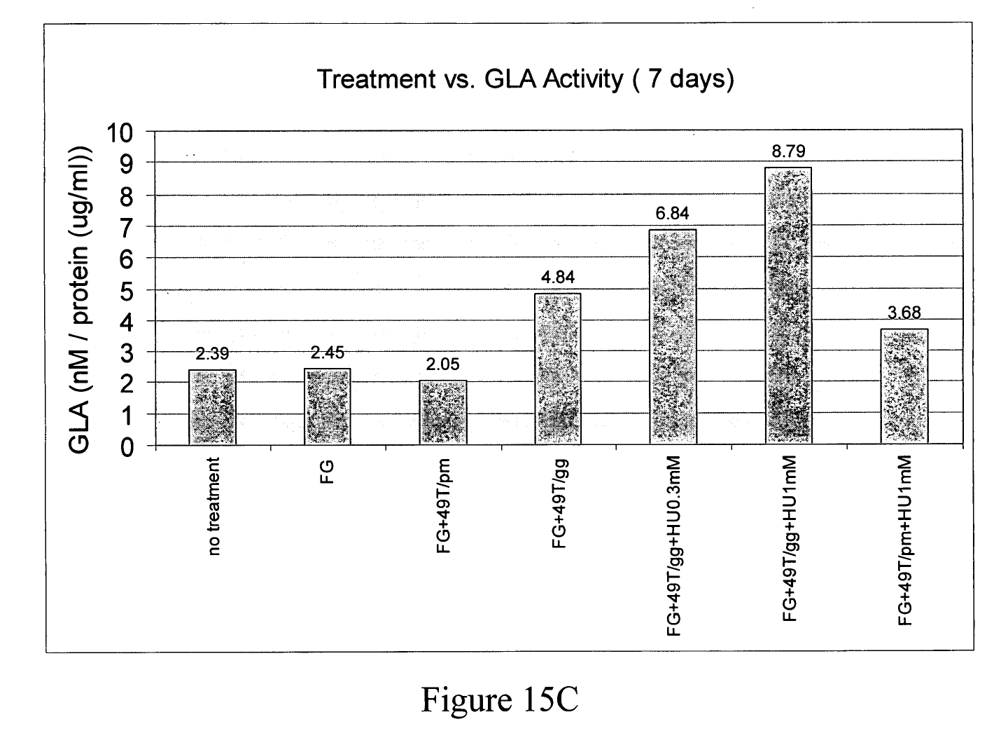

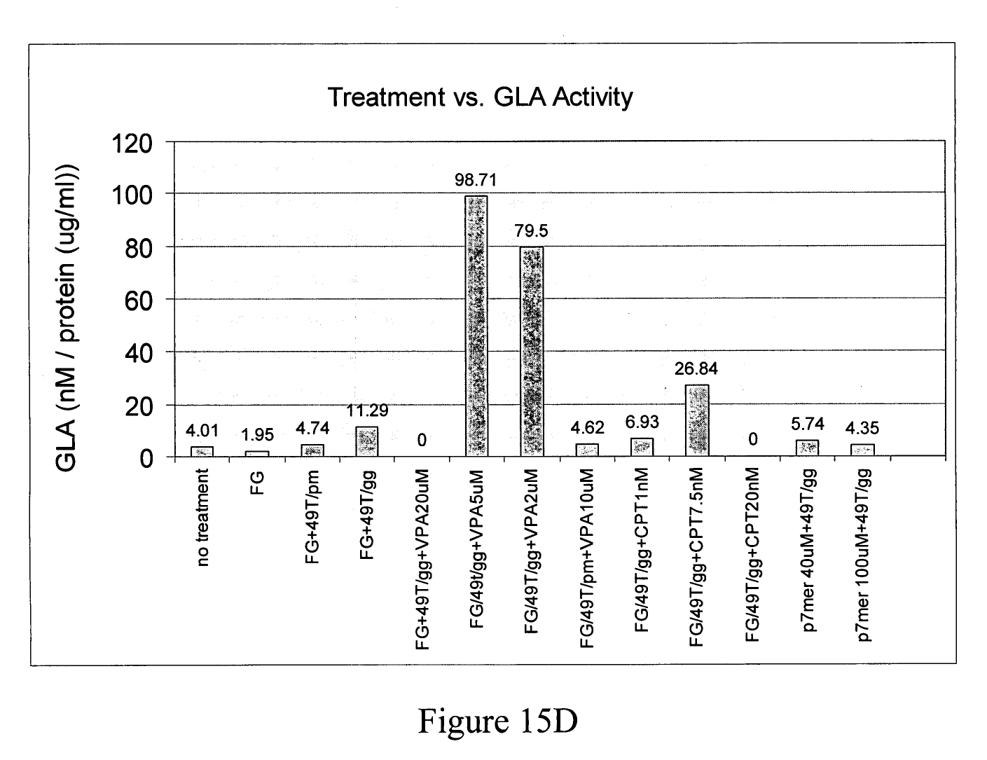

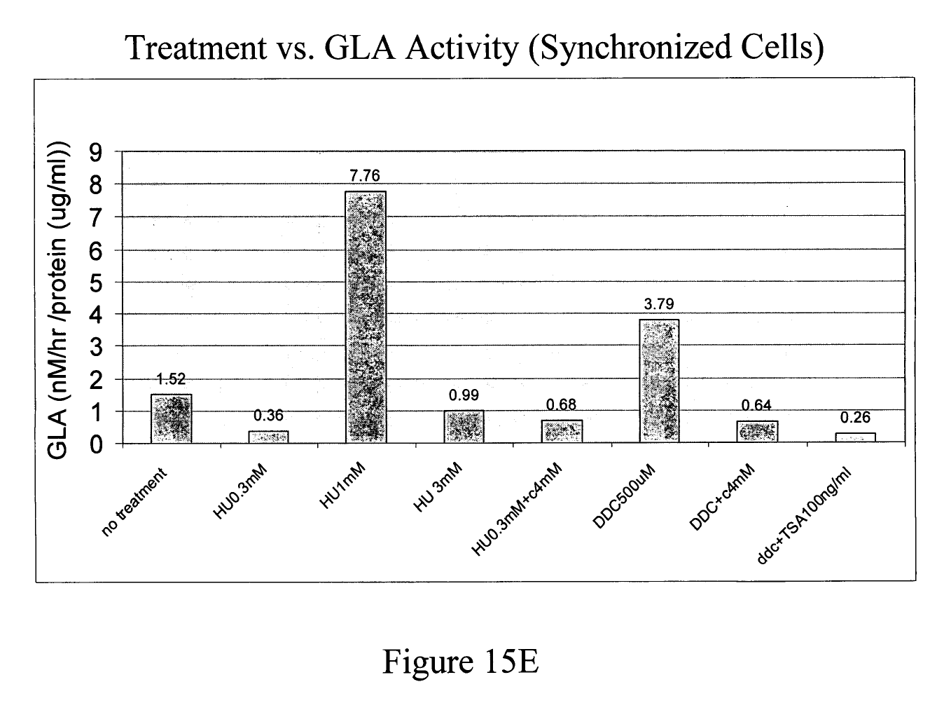

Example 8 illustrates the use of one of the methods of the present invention to repair one such mutant GLA allele to restore GLA function in a test system, the results of which are presented in FIGS. 15A-E. Oligonucleotides are introduced into Fabry's cells by transfection, rather than electroporation, as discussed in Example 8. Unlike most of the previous experiments presented herein, the Fabry's experiments involve treatment with some agents after transfection, during the “recovery period,” rather than before transfection.

FIG. 15A presents an experiment, presented in more detail in Example 8, demonstrating that the oligonucleotide 49T/gg is most effective of the oligonucleotides tested in altering GLA, and that the optimal dosage is 10 μg. The 49NT/pm oligonucleotide is a control oligonucleotide that does not have the capacity to correct the mutation in GLA. Further experiments to correct the mutant Fabry's gene use 10 μg 49T/gg unless otherwise indicated.

FIG. 15B presents data on the effects of several agents, at various concentrations, on correction efficiency. The most dramatic result is the 3.36% correction obtained using 0.3 mM HU, which is over six-fold higher than the control experiment involving treatment with the 49T/gg oligonucleotide, and over 25-fold higher than the untreated control. FG in FIG. 15B refers to a transfection enhancing agent discussed in more detail in Example 8. Other treatments, such as 10 nM CPT, modestly improve correction efficiency. FIG. 15C shows that the results obtained with HU in FIG. 15B are persistent, rather than merely transient, since GLA activity in FIG. 15C is measured seven days after transfection.

FIG. 15D presents data on the effects of several agents, at various concentrations, and several oligos, on GLA activity. A dramatic increase in GLA activity is observed when cells are treated with 2-5 μM VPA in the recovery phase. Such treatment increases GLA activity more than eight-fold compared with cells otherwise treated identically but without VPA, and 25-fold over the activity in untreated cells. CPT (7.5 nM) also more than doubles GLA activity.

FIG. 15E presents results obtained with Fabry's cells that are synchronized by DTB prior to transfection. GLA activity is increased five-fold for synchronized cells treated with 1 mM HU, and approximately two-fold for synchronized cells treated with 500 μM ddC.

### Combinability with Other Methods to Enhance Gene Alteration Efficiency

The methods and kits of the present invention can be combined with one or more other methods of enhancing the efficiency of oligonucleotide-directed alteration of nucleic acid sequence known in the art. Such methods are described, e.g., in copending international patent applications published as WO 02/10364 (“Methods for Enhancing Targeted Gene Alteration Using Oligonucleotides,”); WO 03/027265 (“Composition and Methods for Enhancing Oligonucleotide-Directed Sequence Alteration”); and WO 03/075856 (“Methods, Compositions, and Kits for Enhancing Oligonucleotide-Mediated Nucleic Acid Sequence Alteration Using Compositions Comprising a Histone Deacetylase Inhibitor, Lambda Phage Beta Protein, or Hydroxyurea”), and co-pending U.S. patent application Ser. No. 10/681,074, filed Oct. 7, 2003 (“Methods and Compositions for Reducing Screening in Oligonucleotide-Directed Nucleic Acid Sequence Alteration”), and No. 10/861,178, filed Jun. 4, 2004 (“Reengineering Rad51 for High Efficiency Targeted Nucleotide Exchange”), the disclosures of which are incorporated herein by reference in their entireties.

In one exemplary embodiment, the additional method of enhancing gene alteration efficiency is the addition of a histone deacetylase (HDAC) inhibitor, e.g. trichostatin A (TSA), before, during or after oligonucleotide addition. One of skill in the art will appreciate, however, that other HDAC inhibitors may be suitable for these purposes. For example, U.S. Patent Application Publication No. 2002/0143052, the disclosure of which is incorporated herein by reference in its entirety, discloses compounds having HDAC inhibitor activity due to the presence of a zinc-binding moiety. Other examples of HDAC inhibitors suitable for purposes of the invention include butyric acid, MS-27-275, suberoylanilide hydroxamic acid (SAHA), oxamflatin, trapoxin A, depudecin, FR901228 (also known as depsipeptide), apicidin, m-carboxy-cinnamic acid bishydroxamic acid (CBHA), suberic bishydroxamic acid (SBHA), valproic acid (VPA) and pyroxamide. See Marks et al., J. Natl. Canc. Inst. 92(15):1210-1216 (2000), the disclosure of which is incorporated herein by reference in its entirety. Yet other examples of suitable HDAC inhibitors are chiamydocin, HC-toxin, Cyl-2, WF-3161, and radicicol, as disclosed in WO 00/23567, the disclosure of which is incorporated herein by reference in its entirety.

When administering an HDAC inhibitor to cells or cell extracts, the dosage to be administered and the timing of administration will depend on various factors, including cell type. In the case of TSA, the dosage may be 10 nM, 100 nM, 1 μM, 10 μM, 100 μM, 1 mM, 10 mM, or even higher, or as little as 1 mM, 100 μM, 10 μM, 1 μM, 100 nM, 10 nM, 1 nM, or even lower. In the case of HU, the dosage may be 100 nM, 1 μM, 10 μM, 100 μM, 1 mM, 10 mM, 100 mM, 1 M or even higher, or as little as 100 mM, 10 mM, 1 mM, 100 μM, 10 μM, 1 μM, 100 nM, 10 nM, or even lower.

Cells may be grown in the presence of an HDAC inhibitor, and cell extracts may be treated with the HDAC inhibitor for various times prior to combination with a sequence-altering oligonucleotide. Growth or treatment may be as long as 1 h, 2 h, 3 h, 4 h, 6 h, 8 h, 12 h, 20 h, or even longer, including up to 28 days, 14 days, 7 days, or shorter, or as short as 12 h, 8 h, 6 h, 4 h, 3 h, 2 h, 1 h, or even shorter. Alternatively, treatment of cells or cell extracts with HDAC inhibitor and the sequence-altering oligonucleotide may occur simultaneously, or the HDAC inhibitor may be added after oligonucleotide addition.

Cells may further be allowed to recover from treatment with an HDAC inhibitor by growth in the absence of the HDAC inhibitor for various times prior to treatment with a sequence-altering oligonucleotide. Recovery may be as long as 10 min, 20 min, 40 min, 60 min, 90 min, 2 h, 4 h, or even longer, or as short as 90 min, 60 min, 40 min, 20 min, 10 min, or even shorter. Cells may also be allowed to recover following their treatment with a sequence-altering oligonucleotide. This recovery period may be as long as 1 h, 2 h, 4 h, 6 h, 8 h, 12 h, or even longer, or as short as 8 h, 6 h, 4 h, 2 h, 1 h, or even shorter. The HDAC inhibitor may either be present in or absent from the cell medium during the recovery period.

Optimum dosages and the timing and duration of administration of HDAC inhibitors to cells or cell extracts can be determined by routine experimentation. For example, optimized dosage and timing of treatment with an HDAC inhibitor, such as TSA, can be determined using the assay system described in WO 03/075856.

Some embodiments of the present invention involve supplying cells with enzymes involved in homologous recombination or DNA repair in prokaryotic or eukaryotic cells. Proteins involved in DNA repair in prokaryotes include the λ phage annealing protein red β, and in eukaryotes such proteins include members of the Rad52 epistasis group. Other embodiments involve treatment of cells with agents that alter the levels of such enzymes. In still other embodiments, cells are treated with DNA damaging agents to induce homologous recombination pathways.

Additional embodiments of the present invention contemplate supplying the cells with vectors designed to improve gene editing and repair in addition to the supply of sequence-altering oligonucleotides as described herein. These vectors shall be referred to herein throughout as “Gene Repair Vectors”. Some examples of gene repair vectors include, but are not limited to, PCR fragments, viruses that produce single-stranded DNA which then directs gene editing, double-stranded DNA fragments which produce molecules that promote gene editing, plasmid molecules which are designed to promote gene editing, and RNAis or siRNAs used to inhibit proteins to promote gene repair. The gene repair vectors can be added to the cells exogenously by any method known in the art. Some examples of the use of such gene repair vectors can be found in the following references, the disclosures of which are incorporated herein by reference in their entirety: Kay et al., Viral Vectorsfor Gene Therapy: The Art of Turning Infectious Agents into Vehicles of Therapeutics, Nature Publishing Group (2001); Colosimo et al., Targeted Correction of a Defective Selectable Marker Gene in Human Epithelial Cells by Small DNA Fragments, Molecular Therapy, Vol. 3, No. 2 (February 2001); Majumdar et al., Gene Targeting by Triple Helix-Forming Oligonucleotides, Ann. N.Y. Acad. Sci., 1002: 141-153 (2003); Majumdar et al., Cell Cycle Modulation of Gene Targeting by a Triple Helix-Forming Oligonucleotide; The Journal of Biological Chemistry, Vol. 278, No. 13, pp. 11072011077 (March 2003); H. D. Nickerson and W. H. Colledge, A Comparison of Gene Repair Strategies in Cell Culture Using a lacZ Reporter System, Gene Therapy, 10, 1584-1591 (2003); P. A. Olsen et al. Branched Oligonucleotides Induce in vivo Gene Conversion of a Mutated EGFP Reporter, Gene Therapy, 10, 1830-1840 (2003); H. Nakai et al., Pathways of Removal of Free DNA Vector Ends in Normal and DNA-PKcs Deficient SCID Mouse Hepatocytes Transduced with rAAV Vectors, Human Gene Therapy, Vol. 14, No. 9, 871-881 (June 2003).

### Kits/Research Tools

Further embodiments of the invention are compositions and kits comprising a cell, cell-free extract, or cellular repair protein, at least one agent selected from those disclosed herein as increasing the efficiency of OGDA (or their equivalents), and at least one sequence-altering oligonucleotide which is capable of effecting a desired sequence alteration at a nucleic acid target site. In some embodiments the compositions or kits comprise a nucleic acid molecule comprising a nucleic acid target sequence for the at least one oligonucleotide, which sequence alteration confers a selectable phenotype.

A cell, cell-free extract, or cellular repair protein for a composition or kit of the invention may be derived from any organism. Compositions and kits of the invention and may comprise any combination of cells, cell-free extracts, or cellular repairs proteins and the cells, cell-free extracts, or cellular repair proteins may be from the same organism or from different organisms. Cellular repair proteins that may be used include, for example, proteins from the RAD52 epistasis group, the mismatch repair group, or the nucleotide excision repair group. In some embodiments, the cell, cell-free extract, or cellular repair protein is or is from a eukaryotic cell or tissue. In some embodiments, the eukaryotic cell is a fungal cell, e.g. a yeast cell. In other embodiments, the cell is a plant cell, e.g., a maize, rice, wheat, barley, soybean, cotton, potato or tomato cell. Other exemplary plant cells include those described elsewhere herein. In some embodiments, the kits comprise at least one agent selected from those disclosed herein as increasing the efficiency of OGDA (or their equivalents). In some embodiments such kits also include instructions for use.

Other embodiments of the invention relate to kits comprising a nucleic acid molecule the nucleic acid sequence of which has been altered according to a method of the invention or using a composition or kit of the invention. In some embodiments, the invention relates to kits comprising a cell comprising a nucleic acid molecule the nucleic acid sequence of which has been altered according to the methods of the invention or using a composition or kit of the invention. In some embodiments, the nucleic acid molecule is selected from the group consisting of: mammalian artificial chromosomes (MACs), PACs from P-1 vectors, yeast artificial chromosomes (YACs), bacterial artificial chromosomes (BACs), plant artificial chromosomes (PLACs), plasmids, viruses or other recombinant vectors.

### Pharmaceutical Compositions

Purified oligonucleotide compositions may be formulated in accordance with routine procedures as a pharmaceutical composition adapted for bathing cells in culture, for microinjection into cells in culture, and for intravenous administration to human beings or animals. Typically, compositions for cellular administration or for intravenous administration into animals, including humans, are solutions in sterile isotonic aqueous buffer. Where necessary, the composition may also include a solubilizing agent and a local anaesthetic such as lignocaine to ease pain at the site of the injection. Generally, the ingredients will be supplied either separately or mixed together in unit dosage form, for example, as a dry, lyophilized powder or water-free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection” or saline. Where the composition is to be administered by injection, an ampule of sterile water for injection or saline may be provided so that the ingredients may be mixed prior to administration.

Pharmaceutical compositions of this invention comprise the oligonucleotides used in the methods of the present invention and pharmaceutically acceptable salts thereof, with any pharmaceutically acceptable ingredient, excipient, carrier, adjuvant or vehicle.

The oligonucleotides of the invention are preferably administered to the subject in the form of an injectable composition. The composition is preferably administered parenterally, meaning intravenously, intraarterially, intrathecally, interstitially or intracavitarilly. Pharmaceutical compositions of this invention can be administered to mammals including humans in a manner similar to other diagnostic or therapeutic agents. The dosage to be administered, and the mode of administration will depend on a variety of factors including age, weight, sex, condition of the subject and genetic factors, and will ultimately be decided by medical personnel subsequent to experimental determinations of varying dosage as described herein. In general, dosage required for targeted nucleic acid sequence alteration and therapeutic efficacy will range from about 0.001 to 50,000 μg/kg, e.g. between 1 to 250 μg/kg of host cell or body mass or a concentration of between 30 and 60 μM.

For cell administration, direct injection into the nucleus, biolistic bombardment, electroporation, liposome transfer and calcium phosphate precipitation may be used. In yeast, lithium acetate or spheroplast transformation may also be used. In one method, the administration is performed with a liposomal transfer compound, e.g., DOTAP (Boehringer-Mannheim), Lipofectamine™ 2000 (Invitrogen™) or an equivalent such as lipofectin. The amount of the oligonucleotide pair used, for example, is about 500 nanograms in 3 micrograms of DOTAP per 100,000 cells or about 1 microgram with 1 microliter Lipofectamine™ 2000 per 1,000,000 cells. For electroporation, between 20 nanograms and 30 micrograms of oligonucleotide per million cells to be electroporated is an appropriate range of dosages which can be increased to improve efficiency of genetic alteration upon review of the appropriate sequence according to the methods described herein.

If compatible, agents that enhance ODSA according to the methods of the present invention may be incorporated into, or compounded with, purified oligonucleotide pharmaceutical compositions to increase the efficiency of gene alteration.

### EXAMPLES

In order that this invention may be better understood, the following examples are set forth. These examples are for purposes of illustration only, and are not to be construed as limiting the scope of the invention in any manner.

### Example 1

### DLD-1-1 System for Quantifying Gene Alteration in a Mammalian Cell Line

Cell line and culture conditions: DLD-1 cells are obtained from ATCC (American Type Cell Culture, Manassas, Va.). DLD-1 integrated clone 1 (DLD-1-1) is obtained by integration of the vector pEGFP-N3 containing a single point mutation (TAG) in the eGFP gene, as described in copending U.S. patent application Ser. No. 10/986,418, filed Nov. 10, 2004 (“Mammalian Cell Lines for Detecting, Monitoring, and Optimizing Oligonucleotide-Mediated Chromosomal Sequence Alteration”). Cells are grown in RPMI 1640 medium with 2 mM glutamine, 4.5 g/L glucose, 10 mM HEPES, 1 mM sodium pyruvate and supplemented with 10% FBS. Cells are maintained at 5% CO₂, 37° C. and under selection in 200 μg/ml G418 (Gibco, Invitrogen Co., Carlsbad, Calif.).

eGFP gene correction: Cells grown in complete medium supplemented with 10% FBS are trypsinized and harvested by centrifugation. The cell pellet is resuspended in serum-free medium at a density of 1×10⁶ cells/100 μl and transferred to a 4 mm gap cuvette (Fisher Scientific, Pittsburgh, Pa.). The oligonucleotide is then added at a concentration of 4 μM and the cells are electroporated (LV, 250V, 13 msec, 2 pulses, 1 second interval) using a BTX ECM830 apparatus (BTX, Holliston, Mass.). The cells are then transferred to a 60 mm dish containing fresh medium supplemented with 10% FBS and incubated for 48 hrs at 37° C. before harvesting for FACS analysis.

Flow cytometry analysis: eGFP fluorescence of corrected cells is measured by a Becton Dickinson FACSCalibur™ flow cytometer (Becton Dickinson, Rutherford, N.J.). Cells are harvested 48 hrs after electroporation and resuspended in FACS buffer (0.5% BSA, 2 mM EDTA, 2 μg/ml propidium iodide in PBS). More specifically, the program is set for the appropriate cell size (forward scatter versus side scatter) and the population of single-cells is gated for analysis. Using the negative control (minus PI, minus GFP) the background fluorescence was set by positioning the cells in the 10¹ decade of the dot plot by adjusting the voltage for FL1 (GFP) and FL2 (PI). The composition is then set for multi-fluorochrome experiments using a GFP control sample containing no PI and increasing the compensation to bring the signal toward the FL1 parameter. Finally, the last control, PI and no GFP is used to increase the compensation to bring the signal toward the FL2 parameter. Samples of 50,000 cells each are analyzed and those cells being GFP positive and PI negative are scored as corrected cells.

The percentage of converted cells in a whole population is then calculated by CellQuest™ (Becton Dickinson) and GFP/PI programs. The correction efficiency is determined by dividing the number of eGFP positive cells by the number of cells analyzed in each experiment (usually 50,000 cells). Each clone is tested three times to determine the standard deviation of the correction efficiency, with standard deviation calculated using Microsoft Excel. Control experiments, based on confocal images of cells obtained 2 days and 8 days after electroporation, show that gene alterations are inheritable, as discussed in copending U.S. patent application Ser. No. 10/986,418 (see supra).

FIG. 3 shows histograms (dot plots) from flow cytometric analysis for DLD-1-1 either untreated or treated with correcting oligonucleotide EGFP3S/72NT, with propidium iodide fluorescence on the Y axis and eGFP fluorescence on the X axis. The dot plots are divided into four quadrants, as follows. LR (low right quadrant): the number of live cells with eGFP expression; LL (low left quadrant): the number of live cells without eGFP expression; UR (upper right quadrant): the number of dead cells with eGFP expression; UL (upper left quadrant): the number of dead cells without eGFP expression. Flow cytometry, which is capable of individually querying cells for fluorescence emission, and is also able to provide group statistics, thus is superior in consistency to earlier assays using confocal microscopic examination. In addition, levels of eGFP that are detectable by FACS are often not detectable by confocal visualization.

For cell cycle analysis, 1×10⁶ cells are plated 24 hrs before the treatment with drugs and after 24 hrs of treatment, cells are trypsinized, resuspended in 300 μl cold PBS and fixed by adding 700 μl cold ethanol. Cells are then resuspended in 1 ml of PBS containing 500 g/ml RNaseA and 2.50 g/ml propidium iodide and analyzed for DNA content. The number of cells possessing actively replicating forks is determined by BrdU staining (In Situ Cell Proliferation Kit, FLUOS, Roche Diagnostics, Indianapolis, Ind.) following manufacturers suggestions.

Pulsed-field gel electrophoresis: Twenty-four hours before treatment with HU or VP16, 1×10⁶ cells are plated in tissue culture flasks, followed by induction of DNA damage with HU or VP16 for 24 hrs. The cells are released by trypsinization and melted in the agarose inserts. The agarose inserts are incubated in 0.5M EDTA—1% N-laurosylsarcosine—proteinase K (1 mg/ml) at 50° C. for 48 hrs and then washed four times in TE buffer prior to loading on a 1% agarose gel (Pulse-Field Certified Agarose, Bio-Rad, Hercules, Calif.) and DNA separation by pulsed-field gel electrophoresis is carried out for 24 hrs (Bio-Rad, 120° field angle, 60 to 240s switch time, 4 V/cm). The gel is subsequently stained with ethidium bromide and analyzed with AlphaImager™ 2200. (Alpha Innotech Corp., San Leandro, Calif.).

Results: Gene repair activity is assayed using a mutant eGFP gene as a target. The wild type gene is mutated at amino acid 67 in the chromophore region so that no green fluorescence is observed when it is expressed. The mutation creates a stop codon (TAG) at a site that originally encoded a tyrosine residue (TAC). The eGFP gene is integrated into DLD-1 cells using a pEGFP-N3 vector generating a clonal cell line known as DLD-1-1 (clone-1) (Hu et al. submitted). These cells contain 2-4 copies of the mutant eGFP gene but do not produce functional eGFP (see below).

The experimental strategy involves the introduction of oligonucleotides into DLD-1-clone 1 cells by electroporation followed by phenotypic readout of the corrected eGFP gene, 48 hours later. The correcting oligonucleotide (EGFP3S/72NT) is 72 bases in length (72-mer), complementary to the non-transcribed strand of the mutant eGFP gene but designed to create a single mismatch in the third base of codon 67 (see FIG. 1A). It directs conversion of a TAG→TAC codon which enables phenotypic expression of eGFP, which can be detected by FACS. FIG. 1B outlines the sequence of the target gene, the 72-mer and a nonspecific 74-mer used as a control. The time of addition of certain agents such as hydroxyurea or VP16, relative to the timing of electroporation of the oligonucleotide is described below (see FIG. 2).

FIG. 3 demonstrates the usefulness and validity of the eGFP system. Clone 1 cells are electroporated with either EGFP3 S/72NT or Hyg3 S/74NT and the level of gene correction is measured 48 hours later by FACS analysis. Approximately 1.2% of the cells treated with EGFP3S/72NT score positive for eGFP expression but the frequency of correction in any given experiment is observed to vary from 0.8% to 1.4%. No green fluorescence is observed in the control (lower right quadrant) when the population of cells is treated with the nonspecific oligonucleotide Hyg3S/74NT or with a completely complementary oligonucleotide (data not shown). As displayed in FIG. 1B, Hyg3S/74NT contains no direct sequence complementarity to the mutant eGFP target site.

### Example 2

### Modulation of Cell Cycle to Increase Sequence Alteration Efficiency

The effect of cell cycle on the efficiency of ODSA is assessed by synchronizing a population of DLD-1-1 cells with mimosine, which arrests cells in early S phase, and serum starvation. Cells are seeded at a density of 0.8×10⁶ per 100 mm dish, attached for 20 hours and then cultured in RMPI-1640 medium containing 0.2% fetal bovine serum (FBS). These cells are grown for 48 hours followed by treatment with 0.1 mM mimosine (Sigma, St. Louis, Mo., USA) for 20 hours. Cell are washed twice with PBS and released at various times into fresh medium complemented with 10% FBS before electroporation. Cells are rinsed once with PBS, trypsinized and harvested by centrifugation and resuspended in PBS containing 10 μg/ml propidium iodide, 0.03% Triton-100 and 1 mg/ml RNase. Cells are incubated at room temperature for 1 hour before the measurement of DNA content by FACSCalibur™ flow cytometer. The percentage of cells at various stages of the cell cycle is determined by ModFit L™ software (Verity Software House, Inc., Topsham, Me., USA).

Oligonucleotide-Directed Sequence Alteration: The resulting synchronized populations of cells, and asynchronous controls, are grown in complete medium supplemented with 10% FBS and trypsinzed and harvested by centrifugation at 1500 rpm for 5 minutes. The cell pellet is resuspended in fresh serum-free medium at a density of 2×10⁶ cells/100 μl. The entire cell suspension is mixed with 20 μg of EGFP/72NT and transferred into a 4 mm gap cuvette (Fisher Scientific, Pittsburgh, Pa., USA) followed by electroporation with two 250V pulses, each 13 ms in duration, with one second between pulses, unipolar.

Flow Cytometric Analysis: Cells with corrected eGFP genes exhibit fluorescence detectable by flow cytometry. Cells are washed once with PBS, collected by trypsinization, centrifuged, and resuspended in 1 ml FACS buffer (0.5% BSA, 2 mM EDTA, pH 8.0, 2 μg/ml propidium iodide). Cells are incubated at room temperature for 30 min. The proportion of converted cells are measured using a Becton Dickinson FACSCalibur™ flow cytometer (Becton Dickinson, Rutherford, N.J., USA). Frequency of converted cells are calculated by CellQuest™ and GFP/PI programs.

FIG. 4 presents histograms showing the distribution of cells in the cell cycle as a function of the time after release from arrest, which is also the time of electroporation. Each panel plots the number of cells observed as a function of the intensity of propidium iodide fluorescence from that cell. Gene correction is measured 48 hours afterwards. Numerical summaries of the results are presented in the table in FIG. 4. The correction efficiency (C.E.) increases 2.5-fold, from 0.92% to 2.29% as the proportion of cells in S phase increases 2.5-fold, from 35% (asynchronous cells) to 86% (8 hours after release from growth arrest). These results reveal a strong correlation between the percentage of cells in S phase and the efficiency of gene repair.

### Example 3

### Gene Repair in a Mammalian Cell Line Treated with HU, VP16 or Thymidine

DLD-1-1 cells are subjected to gene repair protocol outlined in Example 1, except that the cells are pre-treated with either HU, VP16 or thymidine as follows.

Treatment of DLD-1-1 cell cultures with hydroxyurea, VP16 or thymidine: Cells are seeded at a density of 0.8×10⁶ cells 24 hrs before addition of hydroxyurea (HU) (0, 0.3, 1, 2, 5 mM) or eptopside (VP16)(0, 0.5, 1, 3, 10 μM). A 500 mM HU (Acros Organics, Morris Plains, N.J.) stock solution is prepared in distilled water and a 50 mM stock solution of VP16 (Sigma, St. Louis, Mo.) is prepared in DMSO (100%). Hydroxyurea and VP16 are added to the cells at the indicated concentrations, and the time of treatment is varied from 0-45 hrs and 0-24 hrs, respectively. Unless otherwise indicated, cells are treated with HU or VP16 for 24 hours.

Cells are then electroporated in the presence of the correcting oligonucleotide EGFP3S/72NT, and analyzed by FACS to determine the percentage exhibiting eGFP fluorescence, which reflects the percentage of cells undergoing gene repair, as described in Example 2.

DNA Damage Caused by HU and VP16: The concentration range of HU and VP16 used in our experiments have been reported previously to induce DNA damage, most often double-stranded DNA breaks. These conclusions, however, were drawn from experiments conducted in other cell lines, not the DLD-1 line. Thus, we monitor the formation and/or accumulation of double strand breaks in DLD-1 cells by pulse-field gel electrophoresis (PFGE) to assess the degree of DNA damage resulting from the addition of HU and/or VP16. Cultures of asynchronously growing DLD-1 cells are incubated with varying concentrations of HU or VP16 for 24 hours and DNA breakage is then assessed by PFGE. As shown in FIG. 5, a progressive increase in the amount of damaged DNA, as a function of HU or VP16 concentration, is observed. Importantly, double strand breaks are found at the concentration of HU and VP16 that have been shown coincidentally to stimulate the frequency of gene repair.

Increased Correction Efficiency in the Presence of HU and VP16: FIG. 6 shows that DLD-1-1 cells treated with 1 mM HU undergo gene repair at a frequency of 2.2%, compared with a frequency of only approximately 1% in untreated cells. HU is known to induce double strand breaks at 1 mM, consistent with the hypothesis that DNA damage is responsible for the increased gene repair efficiency. FIG. 6 also shows that DLD-1-1 cells treated with 3 μM VP16 undergo gene repair at a frequency of over 6%. The asterisks in FIGS. 6 and 7 indicate points that exhibit a statistically significant difference from the (zero) control: one asterisk (*)=p value<0.05, whereas two asterisks (**)=p value<0.01.

As illustrated in the lower panels of FIG. 6, FACS results on populations DLD-1-1 cells stained with propidium iodide indicate that viability is moderately reduced when cells are treated with HU or VP16 prior to electroporation.

In an experiment to assess the optimal HU treatment time, cells were grown for 24 hours, and then treated with 1 mM HU for 15, 24, 30, 35, 40 or 45 hours. As shown in FIG. 7, gene repair efficiency increases as HU treatment increases up to 30-35 hours, and then plateaus. An analogous experiment with 3 μM VP16 shows that the efficiency of correction begins to plateau around 12 hours.

Cell Cycle Modulation by HU and VP16: The effects of HU and VP16 on the distribution of the treated DLD-1-1 cells through the cell cycle, in the absence of any other attempt to modulate cell cycle (e.g. mimosine treatment or DTB as discussed further in Example 4), are determined as follows. DLD-1-1 cells are treated with 1 mM HU, 3 μM VP16, or left untreated, for 24 hours. The resulting cells are then either analyzed by FACS, or the percentage of cells in S phase is determined by BrdU incorporation. FIG. 8 presents the results of both sets of experiments. The FACS results show that HU treatment causes a substantial shift of cells into the leftmost peak, representing cells in S phase, and that VP16 treatment causes a more modest shift. The BrdU data also show that HU increases the percentage of cells in S phase from 49% to 77%, and that VP16 increases the percentage to 56%.

### Example 4

### The Effect of Cell Cycle on Gene Repair in a Mammalian Cell Line Treated with HU, VP16 or Thymidine

In one set of experiments, DLD-1-1 cells are subjected to gene repair protocol outlined in Example 3, except that cells are synchronized in the cell cycle using a double thymidine block (DTB) protocol prior to electroporation.

Double Thymidine Block: Cells are synchronized in G1 or at the G1/S border by a double thymidine block. Twenty-four hours prior to the addition of any agent (HU, etc.), cells are plated at a density of 0.5×10⁶ cells per 100 mm dish, followed by incubation in 2 mM thymidine (Sigma) for 16 hrs, washed and released in fresh medium for 10 hrs, then incubated in 2 mM thymidine for an additional 15 hrs.

Treatment of DLD-1-1 cell cultures with hydroxyurea, VP16 or thymidine: After releasing the DLD-1-1 cells from the double thymidine block by washing out the second thymidine block, the cells are incubated for an additional 24 hours in the presence of 1 mM HU, 3 M V16 or 10 mM thymidine. FACS is then used to analyze 50,000 cells from each population to determine the percentage of cells expressing functional eGFP, as described in Example 1.

FIG. 9B shows the correction efficiency as a function of treatment for both synchronous (double thymidine blocked) and asynchronous populations of cells. The control population of cells is electroporated with EGFP3S/72NT in the absence of any other agent, and give a correction efficiency of approximately 1.5% in asynchronous cells, or approximately 2.5% in synchronous cells. HU increases the correction efficiency of asynchronous cells, from 1.5% to almost 3%, and it stimulates gene correction even more significantly in the synchronized culture, raising the frequency from approximately 2.5% to greater than 9%. In contrast, synchronization does not enhance correction efficiency for VP16-treated cells. Thymidine does not enhance correction efficiency in asynchronous cells but increases efficiency to over 7% in synchronous populations of cells.

### Example 5

### The Effect of MMS and Bleomycin on Gene Repair

The effects of MMS and bleomycin on oligonucleotide-directed gene alteration are determined as follows. DLD-1-1 cells are seeded in 100 mm dishes at 2×10⁶ cells per plate and immediately treated with 0.2 μM MMS or 0.75 μM bleomycin. The cells are then grown for 24 hours, until approximately 50% confluent, and washed twice with PBS. A portion of each population of cells is removed for DNA analysis by pulsed-field electrophoresis, as described in Example 1. The correcting oligo EGFP/72NT (10 μg) is then added and the cells are electroporated as in Example 3. Cells are then analyzed to determine the percentage of cells with corrected eGFP genes as described in Example 3.

FIG. 10 shows lower bands in lanes with DNA from bleomycin and MMS-treated cells, representing DNA fragments resulting from double stranded breaks, showing that MMS and bleomycin effect DNA damage on DLD-1-1 cells under the conditions of this assay. FIG. 11A presents the gene correction results in both graphical and tabular form. MMS treatment doubles correction efficiency compared to the non-MMS treated control, and vastly more than cells with oligo treatment. Cell death is not increased by MMS treatment under the conditions of the assay.

Further experiments are performed similarly to the MMS experiments reported supra, except that 4 mM caffeine is used in place of, or in addition to, MMS. The results show that caffeine is ineffective at increasing the efficiency of gene repair when used alone, and is capable of completely suppressing the enhancement otherwise caused by MMS.

### Example 6

### The Effect of ddC and Caffeine on Gene Repair

The effects of dideoxycytidine (ddC) and caffeine on gene alteration efficiency are examined by adding ddC and caffeine at different points during the standard gene alteration procedure using DLD-1-1 cells and comparing the results. Caffeine is either included during a 24 hr pre-incubation, and washed away prior to electroporation, or caffeine is added only after electroporation. When ddC is added, it is added only during the 24 hour pre-incubation.

Mammalian DLD-1-1 cells (further described in Example 1) are maintained in RPMI+, with G418 added to 200 μg/ml at each successive passage of the cells, except that G418 is not present when cells are electroporated or for 24 hours afterwards. RPMI+ comprises RPMI medium 1640 supplemented with 10% fetal bovine serum (FBS), 2 mM L-glutamine, 1 mM sodium pyruvate, 10 mM HEPES and 0.45% D(+)glucose. Cells are grown to ˜90% confluency in one or two 100 mM dishes. Cells are then trypsinized, counted and 1-2×10⁶ cells are placed in a new 100 mM dish (one for each sample). In parallel experiments, ddC is either added to the media to a final concentration of 0, 100, 250, 500 or 750 μM, and the cells are incubated for 24 hrs. In parallel experiments, caffeine (4 mM) is added to the 750 μM ddC-treated cells during this 24 hr incubation. Cells are collected from the plate by trypsinization, spun down, and resuspended in RPMI 1640 (no serum) to a concentration of 2×10⁷ cells per mL.

Electroporation is then performed using a BTX ECM 830 square wave electroporation device. Oligonucleotides to correct the mutation in the eGFP gene are added to the cells prior to electroporation, as discussed supra. Electroporation is performed in 4 mm gap cuvettes, using 2×10⁶ cells in a 100 μl volume. The cells are exposed to two 250V pulses, each lasting 13 msec. After electroporation, 500 μl of RPMI+ is added to the cuvette and the entire contents are transferred to a 60 mM dish containing 2.5 mL of pre-warmed media.

For cells that were not previously treated with caffeine, caffeine is added to the media to a final concentration of 4 mM in the 60 mM dish immediately following electroporation (the “recovery phase”). For cells that were pre-treated with caffeine, there is no addition of caffeine during the recovery phase. After 24 hrs of recovery, the media is changed and caffeine (4 mM final concentration) is added back to the culture. After 48 hrs of recovery, the samples are read by FACS: eGFP fluorescence reflects gene alteration (correction), and propidium iodide (PI) staining reflects cellular viability.

A further parallel set of experiments is performed as describes for caffeine, but with vanillin added to a final concentration of 1 mM in place of 4 mM caffeine. Another parallel set of experiments is performed varying the length of time the caffeine is present in the recovery phase.

FIG. 13A presents a does curve for ddC in the absence of caffeine. The maximal increase in correction efficiency of approximately two- to three-fold is observed at 500 μM ddC.

FIG. 13B shows that caffeine inhibits oligonucleotide-directed gene correction if added prior to electroporation, but that it stimulates correction if added in the recovery phase (i.e. the period after electroporation). The inhibition of gene correction by caffeine is enhanced when combined with ddC during pretreatment. Neither of these effects are seen when 1 mM vanillin is used in place of caffeine, as illustrated in FIG. 13C.

FIG. 13D shows that the longer the treatment of cells with caffeine in the recovery phase, the greater the enhancement of correction efficiency over the range of times tested (i.e. no caffeine treatment, 12 hrs, 24 hrs or 48 hrs.). All experiments in FIG. 13D include 500 μM ddC in the 24 hr pre-incubation except the leftmost data bar, in which there was no ddC pretreatment.

### Example 7

### The Effect of ddC, Caffeine, AraC, Aphidicolin, and p53 on Gene Repair

Addition of Dideoxycytidine Stimulates the Correction Frequency

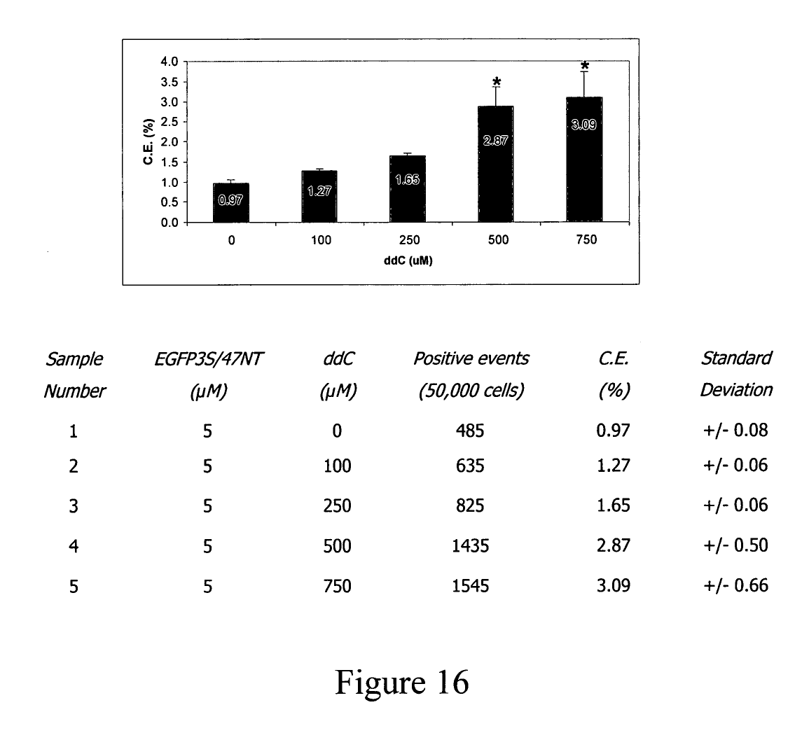

To determine if stalling the replication fork would increase the frequency of targeted gene repair, ddC is added to the cell culture media 24 hrs prior to electroporating the oligonucleotide. Indeed, dideoxycytidine addition causes a dose-dependent increase in oligonucleotide-mediated gene repair (FIG. 16). The most effective concentration for stimulating repair is found to be between 500 μM and 750 μM with higher levels leading to a cellular toxicity (data not shown). The 500 μM and the 750 μM levels are found to have a statistically significant difference from the control (FIG. 16).

Dideoxycytidine but not Dideoxyinosine Results in an Extended S Phase

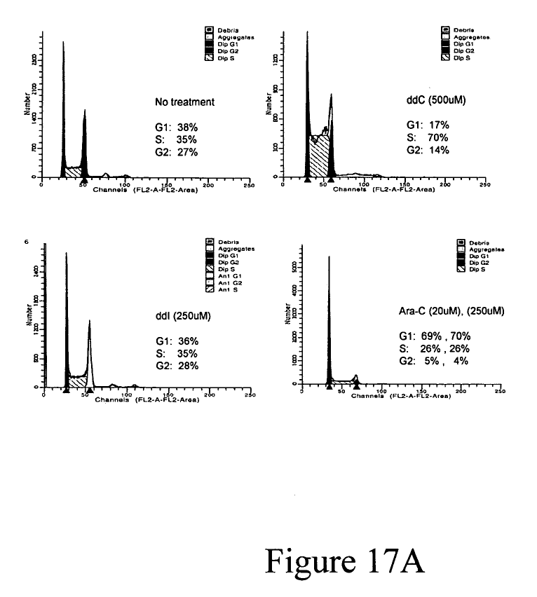

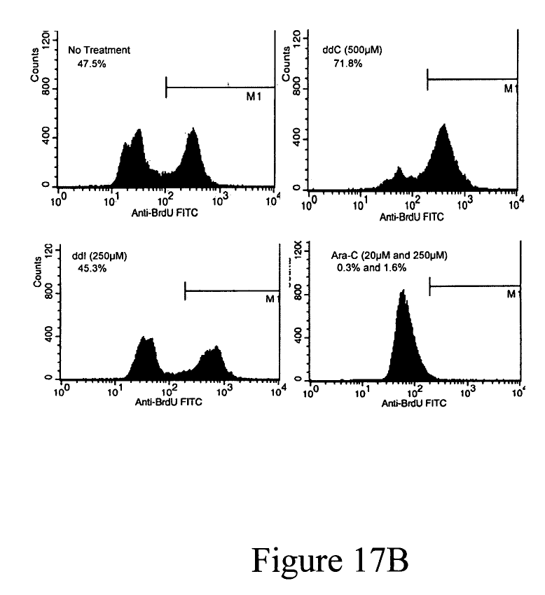

Cell cycle analyses and BrdU incorporation analyses of untreated cells, at the time of oligonucleotide addition, reveal that 35% of cells are in S phase and 47% are actively incorporating DNA bases (active forks) (FIGS. 17A and 17B). Treatment with 500 μM ddC is seen to increase the number of cells in S phase to 70% indicating that ddC extends the time that cells spend in S phase as predicted (FIG. 17A). In addition, evidence that replication fork stalling has occurred is apparent from data generated by the BrdU incorporation experiments (FIG. 17B). The addition of ddC (to 500 μM) leads to an increase in the amount of BrdU uptake (71.8%), a hallmark of the transitory stalling of replication forks.

For comparison purposes, we add dideoxyinosine (ddI), a chain terminator like ddC which must first be metabolized by the cell to its active form 2′,3′-dideoxyadenosine 5′triphosphate (ddATP) prior to being incorporated into the DNA. As measured by cell cycle analysis and BrdU incorporation, ddI does not show an increase in the number of cells in S phase nor does it lead to an elevation in the number of actively replicating forks (FIGS. 17A, 17B). In fact, cells treated with ddI exhibit no difference from non-treated cells in the cell cycle or the BrdU incorporation assays suggesting that ddI has little detectable effect on the replication process.

In contrast, 1-β-D-arabinofuransylcytosine (AraC) is known to be very efficiently incorporated into elongating DNA chains after being converted to its triphosphate form. AraC stops replication fork progression by creating a topoisomerase I cleavage complex on the DNA an adduct that the fork cannot pass by.

Cell cycle analysis of cells treated with AraC reveal that the number of cells in S phase drops from 35% in untreated cells to 26% after a 24 hour treatment with AraC. This reduction does not appear by itself substantial (FIG. 17A); however, when the effect of AraC on the cell population is assayed by BrdU incorporation, the number of cells actively incorporating DNA bases is seen to be essentially zero (FIG. 17B). Thus, AraC is shown to block elongation and prevent restart of fork movement, in contrast to the action of ddC which has a more transient effect on replication and S phase in general.

Neither Dideoxyinosine nor Ara-C Stimulate Gene Repair Activity

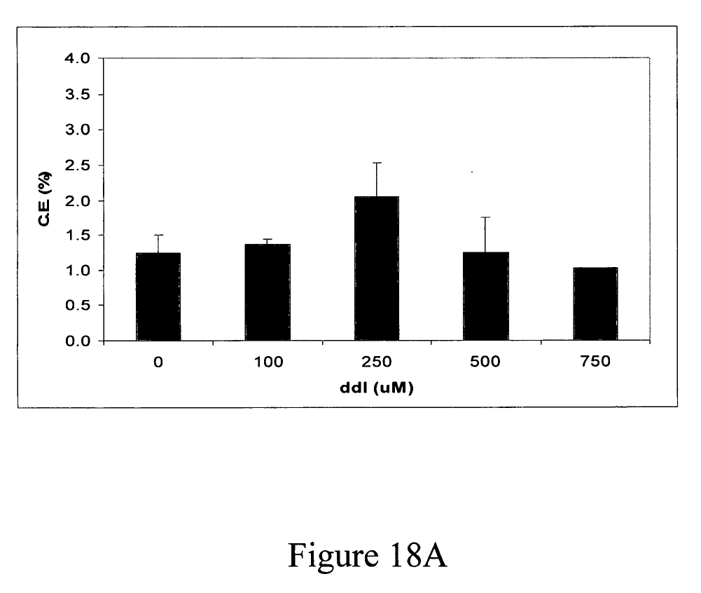

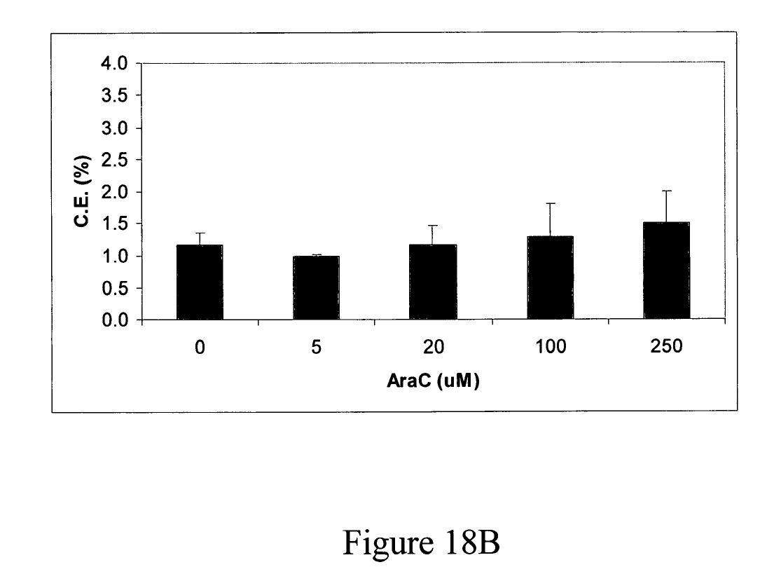

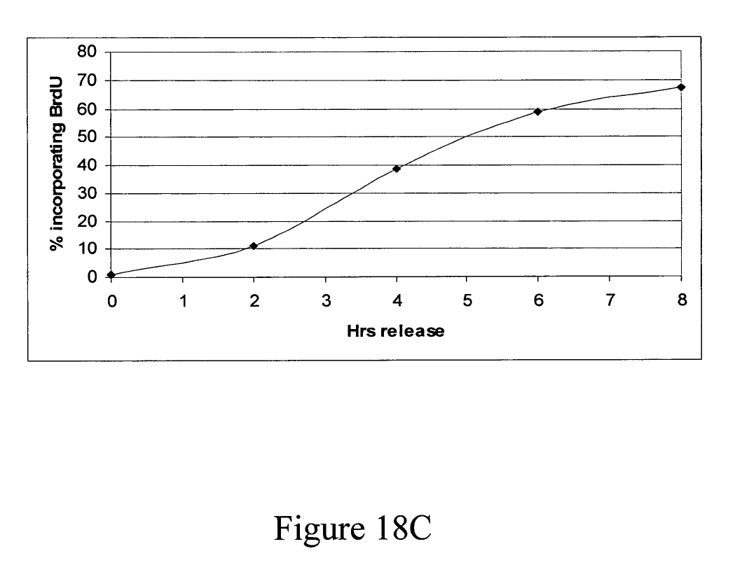

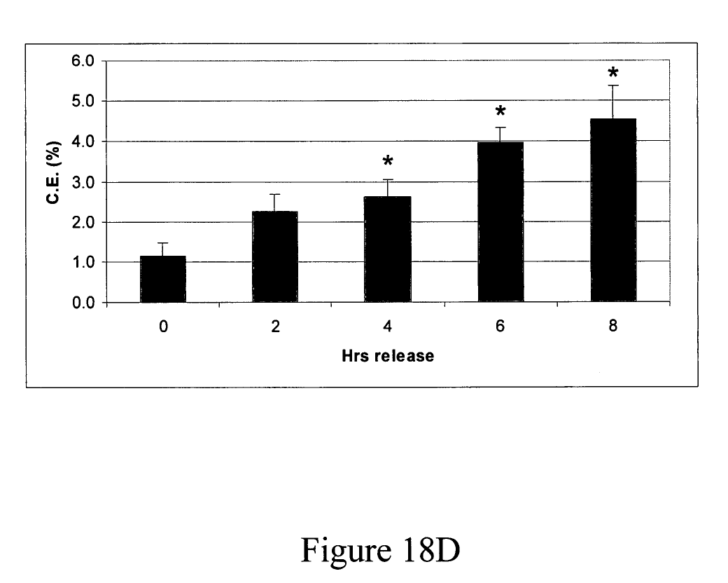

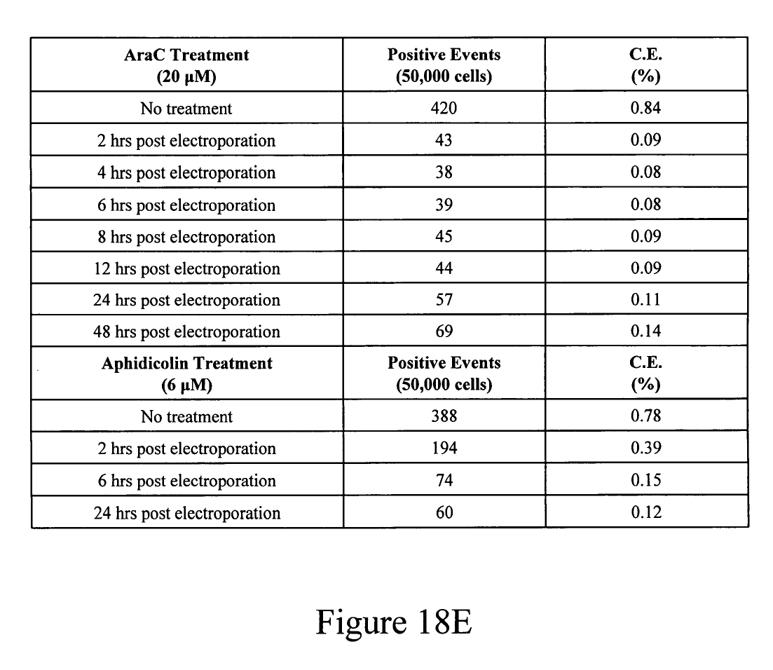

When various dosages of dideoxyinsosine (ddI) are added to the DLD-1 cells, no significant increase in gene correction is observed (FIG. 18A). At the 250 μM ddI level, a statistically insignificant increase in gene repair is observed, suggesting that a small number of replication forks may have been stalled. But, if true, the number appears not to be sufficiently high enough to be detected by cell cycle analyses or by the BrdU incorporation assay.

ddI is known to require intracellular metabolism to its active form, 2′,3′-dideoxyadenosine 5′-triphosphate; if this is not occurring efficiently, incorporation into DNA cannot take place. As a result, the replication fork would neither stall nor slow down. Without intending to be bound by theory, the lack of stimulatory activity of ddI supports the notion that ddC may have a direct and somewhat specific effect on gene repair by being incorporated into the elongating strand.

Likewise, AraC provides no enhancement in correction levels through a broad range of concentrations (5 μM to 250 μM) (FIG. 18B). Cell cycle analyses at 20 μM and 250 μM reveal only a 9% reduction in S phase cells, but the level of BrdU incorporation indicates that DNA synthesis has been halted (FIGS. 17A, 17B). Under these conditions, the cell cycle is effectively arrested and it is likely that cells do not begin DNA synthesis until the drug is washed out. In any case, replication forks are no longer active at the time of electroporation. Therefore, AraC is very effective at stalling replication forks but not at enhancing correction, implying that gene repair activity requires cells in S phase with actively replicating templates at the time of oligonucleotide addition.

Gene Repair Activity is Stimulated Upon Release from the AraC Block of Replication

As stated above, the lack of stimulation observed in cells treated with AraC could be explained by the reduced number of cells passing through S phase or the absence of actively replicating forks. If true, we might predict that a rise in the gene repair frequency would appear if the cells are released from the AraC block and replication forks are allowed to restart prior to the electroporation of the oligonucleotide. We test this prediction by treating the cells with AraC for 24 hrs and then releasing them by washing out the drug at specific times. We measure the level of BrdU incorporation at the time of electroporation and evaluate the frequency of correction 48 hrs later.

As shown in FIGS. 18C and 18D, a small elevation in BrdU incorporation is observed within 2 hrs after release, indicating the regeneration of actively replicating forks. Coincidently, we observe a rise in gene repair activity which then reaches a maximal level 8 hrs post release. The incorporation level and the gene repair level appear to correlate throughout a wide range of timepoints. Thus, an increase in the number of cells in S phase and a rise in the number of actively replicating forks appears to enhance the level of gene repair in DLD-1 cells.

When another inhibitor of DNA replication, aphidicolin is used, the same results are observed (data not shown).

The experimental protocol used thus far in this Example includes a 48 hour recovery period after electroporation to allow for the repair of the mutation and maximal expression of eGFP. This may explain why cells blocked by AraC and electroporated immediately after release (zero-time point in FIGS. 18C and 18D) are still able to undergo gene repair (correction takes place during the 48 hour recovery period).

We wondered whether correction would disappear if replication were blocked in the 48 hrs recovery period.

To address this question, AraC is added in the cultures for various times after electroporation. As seen in the table in FIG. 18E, when AraC is added to the culture for any period of time following electroporation, correction levels drop substantially. Since the number of cells in S phase and the number of cells actively incorporating BrdU correlates with the drop in gene repair activity, we suggest that the most amenable cells to gene repair are those that contain the oligonucleotides during a period of active replication. As there is no difference in correction levels from 2 hrs to 48 hrs of AraC post electroporation, the data suggests that active replication is most important during the time immediately following electroporation.

For maximal levels of gene repair activity therefore, it seems likely that the oligo should be present during periods of active replication. The highest level of correction is attained when either more cells enter S phase simultaneously or cells spend a longer period of time in S phase.

We repeat this experiment using a separate inhibitor of replication elongation which blocks DNA synthesis by a different mechanism. Aphidicolin (6 μM) is added to the reaction at 2, 6 and 24 hrs after electroporation and the frequency of gene repair measured after 48 hrs (FIG. 18E). Consistent with AraC results, the presence of aphidicolin in the recovery/post electroporation phase of the reaction results in a low level of correction.

Wild-Type p53 Blocks Gene Correction Levels Stimulated by ddC, While Mutant p53 Enhances the Frequency.

The tumor suppressor p53 trans-activates a number of genes, regulates cell cycle checkpoints and can act as a trigger-switch for apoptosis. Recently, a suppressive role of p53 on homologous recombination, independent of its transactivation function, has been identified. p53 is recruited to the stalled forks to suppress or impede elevated levels of HR activity that are responding to the disturbance in the replication process. Interestingly, in vitro studies of oligonucleotide-directed repair in MEF cells showed that a p53^{-/-} line exhibited higher correction levels than its p53^{+/+} counterpart. These results indicate that the suppressive activity of wild-type p53 may extend to the gene repair reaction perhaps through its regulatory function of binding to replication forks.

The DNA binding domain of the p53 gene can be mutated so that the p53 protein loses the ability to suppress homologous recombination; it is no longer able to inhibit Rad51-mediated strand exchange and reverse branch migration of stalled replication forks. A few mutant p53 proteins, such as p53(175H) and p53(273P), not only eliminate the suppression of HR but actually stimulate spontaneous, radiation-induced, and replication inhibition-induced HR. Specifically, p53(175H) shows a loss of G1 checkpoint control and the p53(273P) mutation affects the p53⁻ Rad51 interaction.

Stalled replication forks appear to be a stimulant for gene repair activity, and thus we might predict that this effect should be blocked by the action of wild-type p53.

To examine the effects of p53 and its related mutants on the gene repair reaction, we express transiently either wild-type p53 or one of the DNA binding domain mutants [p53(175H), p53(273P)] in the DLD-1 cells.

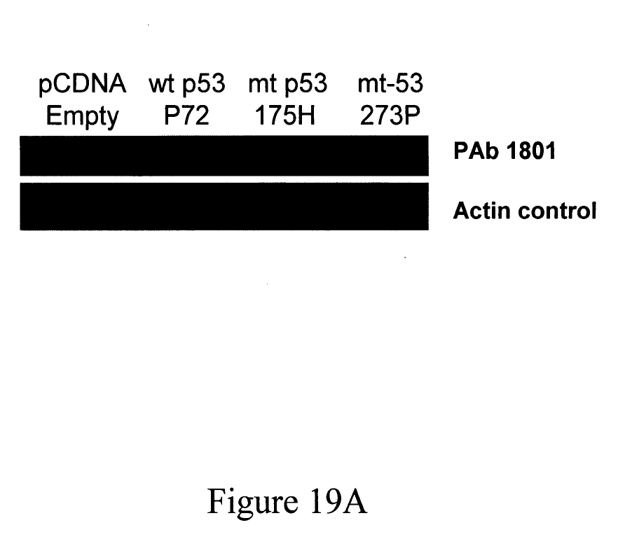

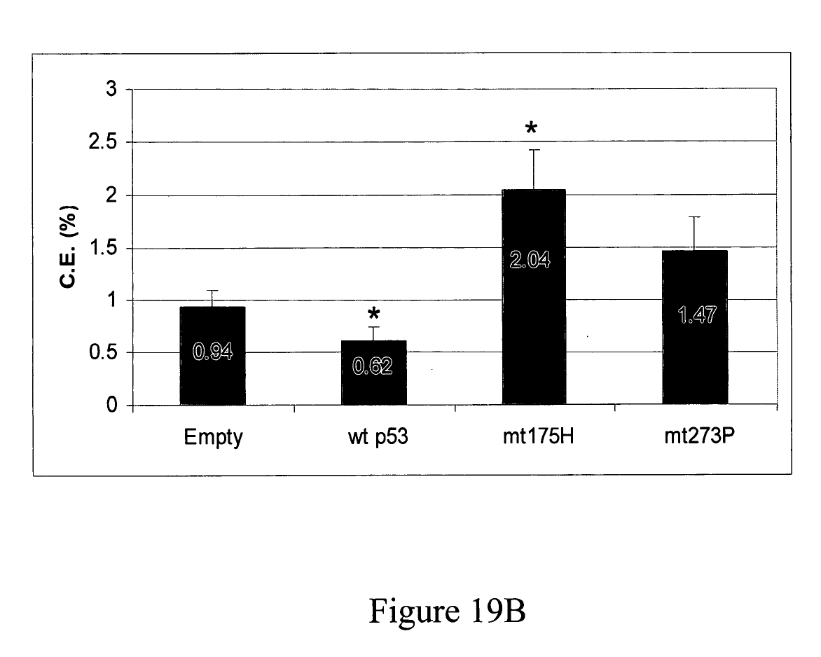

Protein expression of the p53 constructs is confirmed through western blot analysis using the monoclonal p53 antibody, Pab1801, after transfection of the expression constructs. Each of the p53 constructs, being driven by a CMV promoter, express the p53 protein at approximately the same level but beyond that of the endogenous level (FIG. 19A).

When the wild-type expression construct is introduced into cells pretreated with ddC, a decrease in the level of gene correction is observed (FIG. 19B). Overexpression of a mutant p53(273P) shows a slight increase in correction and expression of p53(175H) exhibits an enhancement in the level of correction (FIG. 19B). Since DLD-1 cells contain one copy of the wild-type p53 allele and one copy of a mutant p53 gene (residue Ser241Phe), the expression of wild type p53 from an exogenous source would likely have had a stronger inhibition of correction had the cell line not already had a basal level of wild-type p53 protein.

Despite this, a statistically significant reduction in correction is still observed. In addition, expression of the mutant p53 is able to overcome the effect of the endogenous wild-type p53 levels and enhance gene repair activity through its dominant negative effect.

Taken together, these data suggest that wild-type p53 down-regulates the activity of gene repair in the presence of transiently stalled replication forks.

Caffeine Inhibits Dideoxycytosine-Induced Stimulation of Gene Repair Activity

The mechanism by which ddC acts as a stimulus for gene repair likely involves an extension of S phase, including late S, as well as early G2, stages within which HR pathways exhibit their highest level of activity. The expression of HR proteins is elevated in response to DNA damage at stalled replication forks or lesions that occur naturally during DNA synthesis. Non-homologous end joining (NHEJ), however, can also play a role in the response to altered DNA synthesis processes and its activation is known to proceed that of HR.

Thus, to identify the most predominant pathway governing gene repair events, we expose the cells to inhibitors of NHEJ or HR at doses that are known to block each pathway individually.

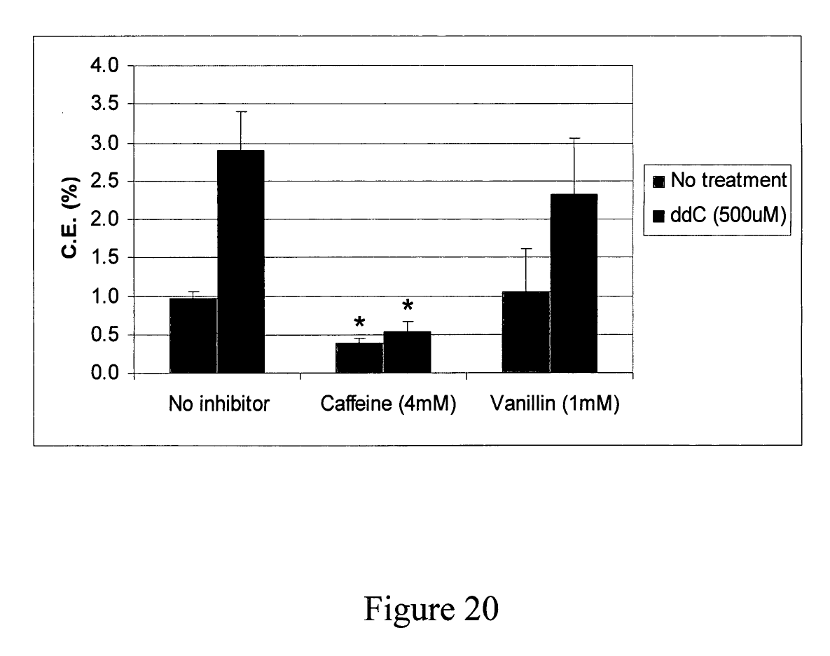

Caffeine, a xanthine derivative and radiosenstizer, inhibits p53 ser-15 phosphorylation by ATM, reducing the level of HR between 60 and 90% while having little effect on NHEJ. Conversely, vanillin blocks the activity of DNA-PK, an essential enzyme in the NHEJ pathway. As seen in FIG. 20, correction levels in cells treated with ddC alone reached levels of 2.9%, consistent with our earlier data. When vanillin is added to the media, the frequency of gene repair is statistically unchanged; however, when caffeine is added to the mix, correction drops substantially (0.6%). Taken together these results indicate that gene repair events directed by oligonucleotides rely more heavily on the activity of the homologous recombination pathway.

Discussion

Without intending to be bound by theory, studies using caffeine and vanillin support the notion that homologous recombination is involved in the gene repair process. Caffeine inhibits ATM kinase activity and the downstream phosphorylation of p53^{ser-15}, thereby inhibiting 60-90% of the cell's HR activities. The pretreatment of cells with caffeine by its addition to the cell culture media not only brings about a reduction in basal repair levels, but also blocks the stimulation in gene repair caused by ddC treatment (FIG. 20). Blocking NHEJ through a pretreatment with vanillin, which inhibits the activity of DNA-PK, does not inhibit gene repair activity, suggesting that HR dominates the gene repair response pathway.

Consistent with this interpretation are the data obtained from the p53 over-expression experiments. Homologous recombination is seen to be inhibited by the overexpression of wild-type p53, which is known to have a high affinity for 3-stranded recombination intermediates, especially those that contain one or more mismatches. Coincidentally, this structure is believed to be a reaction intermediate in the gene repair pathway. p53 also binds to stalled replication forks, stabilizes them, and encourages fork regression, a process that counters the recombination induced replication restart. The over-expression of p53 from an exogenously added plasmid leads to a reduction in gene repair activities. Without intending to be bound by theory, we believe that the reduction in repair activity involves the inhibition of the homologous recombination response to transiently stalled replication forks.

As evidenced by cell cycle analyses and the results of the BrdU uptake experiments, the presence of ddC in the cell culture results in a slowing of the replication process. In effect, treatment with ddC slows the progression of cells through S phase and into G2, and actually hinders the entry of cells into S. Under these conditions, the number of cells that exhibit active replication expands at the time of electroporation and the introduction of the oligonucleotide.

This interpretation fits well with the data obtained by AraC treatment; AraC blocks DNA synthesis, causing an accumulation at the G1/S border or in an early stage of S phase. In this case, fewer cells are in S phase and those that are appear not to contain actively replicating forks. Upon release, the “synchronized” cell population, originally frozen at the G1/S border enter S phase uniformly either at the time of oligonucleotide electroporation or just prior to it. Hence, both ddC and AraC actually lead to the same enhanced population in S phase at the time of electroporation, although they accomplish this by different means.

Furthermore, the results from the AraC experiments suggest that cells bearing actively replicating DNA forks might in fact be more amenable to gene repair or at least are amenable to enhanced levels of gene repair. These results are confirmed by using the replication inhibitor, aphidicolin.

It is possible to increase the frequency of gene repair on therapeutic targets by mobilizing cells into their division cycle.

### Example 8

### Oligonucleotide-Directed Gene Alteration of Fabry's Disease Mutation

Cell line DMN-1, a human fibroblast cell line derived from a human patient with Fabry's disease, is obtained from the National Institutes of Health (NIH). These cells are used to measure the efficiency of correction of a mutant allele of α-galactosidase A (GLA) using methods of the present invention. The specific disease-causing mutation in the Fabry's cell line used herein is A143P, caused by a G→C mutation in the gene. See Branton et al., Medicine (Baltimore) (2002) 81(2): 122-38.

The oligonucleotides synthesized to evaluate the efficacy of the methods of the present invention in correcting the mutant Fabry's disease allele of GLA are presented in Table 1. Oligos are presented 5′→3′ from left to right. Asterisks represent phosphorothioate linkages.

TABLE 1

| | | SEQ ID |
|---|---|---|
| Oligo | Sequence | NO: |
| 49T/pm | G*C*A* GAT GTT GGA AAT AAA ACC TGC | 1 |
| | CCA GGC TTC CCT GGG AGT TTT G*G*A*T | |
| 51NT/pm | T*A*T* CCA AAA CTC CCA GGG AAG CCT | 2 |
| | GGG CAG GTT TTA TTT CCA ACA | |
| | TCT*G*C*A | |
| 49NT/cc | A*T*C* CAA AAC TCC CAG GGA AGC CTG | 3 |
| | CGC AGG TTT TAT TTC CAA CAT C*T*G*C | |
| 49T/gg | G*C*A* GAT GTT GGA AAT AAA ACC TGC | 4 |
| | GCA GGC TTC CCT GGG AGT TTT G*G*A*T | |

The first oligonucleotide, designated 49T/pm (SEQ. ID NO. 1), is a control oligonucleotide that comprises a sequence complementary to the transcribed strand of the gene at all positions and extending both upstream and downstream of the locus of the mutation. Oligonucleotide 51NT/pm is another control oligonucleotide, comprising a sequence perfectly complementary to the non-transcribed strand.

The third oligonucleotide, designated 49NT/cc (SEQ. ID NO. 3), comprises a sequence complementary to the non-transcribed strand of the gene at all positions other than the locus of the mutation and extending both upstream and downstream of the locus of the mutation. For the GLA mutation used in this experiment, a cytosine (C) residue is present at the locus of mutation in the transcribed strand. 49NT/cc has a wild-type C residue at the locus of mutation, giving rise to a C-C base mismatch (rather than a C-G basepair) when annealed to the genomic DNA.

The fourth oligonucleotide, designated 49T/gg (SEQ. ID NO. 4), comprises a sequence complementary to the transcribed strand of the gene at all positions other than the locus of the mutation and extending both upstream and downstream of the locus of the mutation. For the GLA mutation used in this experiment, a guanine (G) residue is present at the locus of mutation in the non-transcribed strand. 49NT/gg has a wild-type G residue at the locus of mutation, giving rise to a G-G base mismatch (rather than a G-C basepair) when annealed to the genomic DNA.

The aforementioned oligonucleotide sequences are exemplary and one of skill in the at would recognize that oligonucleotides comprising other sequences could also be used to effect ODSA in cells harboring mutations in the GLA gene. The mRNA sequence for GLA is available under accession no. NM_{—}000169, and the human gene sequence is available under accession no. U78027, the disclosures of which are incorporated herein by reference in their entireties. Such sequences are readily obtained from public sequence databases, such as Entrez PubMed, accessible at <http://www.ncbi.nlm.nih.gov/entrez>.

With respect to the 49T/gg and 49NT/cc oligonucleotides presented above, and in light of the known sequence for human GLA, additional bases may be added to the 5′ end, the 3′ end, or both, and bases may be deleted from the 3′ end, the 5′ end, or both, to create other oligos capable of effecting gene repair of the A143P mutation of GLA. In one embodiment, the oligonucleotide used to repair the GLA gene comprises 120 nt and has the locus of the relevant mutation near the center of the oligonucleotide. In one embodiment, the sequence of the correcting oligonucleotide is “AGGTTCACAG CAAAGGACTG AAGCTAGGGA TTTATGCAGA TGTTGGAAAT AAAACCTGCG CAGGCTTCCC TGGGAGTTTT GGATACTACG ACATTGATGC CCAGACCTTT GCTGACTGGG”(SEQ. ID NO. 5), wherein the bold base is the mutant base in the specific cell line used in this example. In another embodiment, the oligonucleotide used to repair the GLA gene comprises 17 nt and has the locus of the relevant mutation near the center of the oligonucleotide. In one embodiment, the sequence of the correcting oligonucleotide is “AAACCTGCGCAGGCTTC” (SEQ. ID NO. 6).

Other lengths of oligonucleotide may be used, and the locus of mutation need not be as near the center of the oligonucleotide as in the specific examples listed herein. Correcting oligonucleotides of the present invention may be 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 or more bases long. Correcting oligonucleotides may comprise phosphorothioate linkages, 2′-O-methyl analogs or LNAs or any combination of these modifications.

One of skill in the art would recognize that other mutations that cause Fabry's disease could similarly be repaired using appropriate oligonucleotides, by analogy with the creation of the oligonucleotides listed in this example. The sequence of the gene upstream and downstream of the relevant mutation is obtained using sequence databases, and oligonucleotides are designed that are complementary at those positions but mismatched the locus of mutation, at which position the correcting oligonucleotide comprises the complement of the wild type base at that position, i.e. the correcting oligonucleotide provides a short stretch of the wild type opposite strand paired to the mutant strand in the target DNA. The oligonucleotide can be any length from 17 to 120 nt long.

Both the 49T/cc and 49NT/gg oligonucleotides provide a sequence that can potentially correct the GLA mutation, but the 49T/pm and 51NT/pm oligonucleotides do not, and serve as controls. All four oligonucleotides are used to effect ODSA substantially as described in Example 1, with the exception that cells are transfected with oligonucleotides, rather than electroporated. Oligos are added at 5 μg, 10 μg or 30 μg per reaction. After ODSA is performed, cells are cultured and assayed for GLA activity according to the method of Brady, as described in Medin et al., Proc. Natl. Acad. Sci. (USA) (1996) 93:7917-22, the disclosure of which is incorporated herein by reference in its entirety. The GLA activity is used as a measure of gene correction by comparing the activities in treated versus untreated cultures. The correction efficiency is subsequently confirmed by sequencing of the locus of mutation in a number of treated cells.

As in the previous examples, target cells are treated with HU, VP16 or CPT prior to transfection with the correcting oligonucleotide. Unlike the experiments involving correction of eGFP in DLD-1-1 cells described in Example 1, however, certain of the agents (VPA, caffeine, TSA) in the experiments in this example are not added before transfection, but are instead added only after transfection, during the recovery period.

The means of introducing oligonucleotide into the target cells also differs from previous examples, with transfection in the presence of the transfection enhancing agent FuGENE™ 6 (FG) (Roche Diagnostics Corp., Indianapolis, Ind., USA) being used rather than electroporation. Each transfection reaction includes 100 μl of target cells and 12 μl of FG unless otherwise indicated, despite the fact that FG is not explicitly listed for each datapoint in the relevant figures. Otherwise the procedures used in the DLD-1-1 experiments and the Fabry's experiments are similar.

FIG. 15A shows GLA activity, in units per protein concentration, versus oligonucleotide dose. The figure reflects the results of experiments done in triplicate. The control oligo, 49T/pm, gives the lowest level of GLA activity, representing the base level of GLA activity in cells harboring only the mutant GLA allele. The highest GLA activity is obtained after treatment with 49T/gg, which improves activity up to over four-fold when compared with the control. 49NT/cc is less effective in effecting ODSA than 49T/gg, but nonetheless improved activity over three-fold at some dosages. These results are consistent with other experiments showing a strand bias in ODSA, although the strand most amenable to correction may vary. Optimal correction is obtained with 10 μg of oligo, and decreases significantly at 30 μg.

The same experiment as described with respect to FIG. 15A is repeated in the presence of various agents and treatments to evaluate whether embodiments of the present invention can increase gene alteration (in this example, correction) efficiency. FIG. 15B shows the results of one such series of experiments, which are discussed from left to right. Bars represent the correction efficiencies observed in the various experiments. Unless otherwise indicated, all experiments include 10 μg of 49T/gg in addition to any other agent used to treat the cells.

Cells that receive no treatment, or those that are treated only with FG, exhibit low apparent correction efficiencies. Cells treated with control oligonucleotide 51NT/pm exhibit 0.27% correction, whereas cells treated with with 49T/gg exhibit double that correction efficiency.

Cells that are treated with HU at 0.3 mM along with 49T/gg are corrected at an efficiency of 3.36%, with higher concentrations of HU giving less efficient correction. Addition of VP16 in the concentration range from 1-50 μM has little effect on correction efficiency, regardless of the presence or absence of the correcting oligonucleotide. Camptothecin (CPT) at 10 nM increases correction efficiency to 0.82%, whereas treatment with thymidine over the concentration range from 2-50 mM has little effect on correction efficiency, regardless of the presence or absence of the correcting oligonucleotide. Treatment with a p7-mer known to stimulate DNA repair pathways (pAGT ATG A, where “p” is a 5′ phosphate) modestly improves correction efficiency to 0.62%.

FIG. 15C shows the results of a series of experiments designed to confirm that HU-enhanced gene correction shown in FIG. 15B is not a transient phenomenon. Cells are treated as illustrated in the figure, and then grown for seven days prior to assaying GLA activity. As illustrated in FIG. 15B, treatment with the correcting oligo 49T/gg gives twice the GLA activity as treatment with control oligo 49T/pm, with the data in FIG. 15C showing this to be a non-transient effect. Cells treated with 0.3 mM and 1 mM HU both show a persistent (after seven days) increase in GLA activity of approximately three-fold as compared to untreated cells. When treated with 1 mM HU, the correcting oligo 49T/gg enhances GLA activity over twice as much as the control oligo 49T/pm, showing that the result is sequence-specific.

The results of a further series of experiments are shown at FIG. 15D, where the data are presented as GLA activity as nanomolar (nM) concentration of the product of the GLA assay generated per hour per unit protein concentration (as are the data in FIGS. D and E). Addition of 10 μg of control oligonucleotide 49T/pm, which is perfectly complementary to the transcribed strand of GLA around the locus of mutation, does not appreciably enhance GLA activity compared to the no treatment cells. In contrast, treatment with 49T/gg more than doubles GLA activity. GLA activity is dramatically enhanced by addition of 5 μM VPA during the recovery period, i.e. the period after transfection with 49T/gg, to 98.71, over 20-fold higher than untreated cells. GLA activity is also enhanced by addition of 7.5 nM CPT during recovery to 26.84, approximately six-fold higher than the no treatment control. Both VPA and CPT exhibit non-linear dose response curves, with the highest tested concentrations of each agent giving the lowest GLA activity.

In a further set of experiments on Fabry's cells, the results of which are presented at FIG. 15E, cells are synchronized in the cell cycle using a double thymidine block protocol prior to treatment with oligonucleotides and other agents. Under these conditions treatment with 1 mM HU prior to transfection with the correcting oligonucleotide 49T/gg increases GLA activity five-fold as compared to untreated cells. As observed with in other experiments, the HU dose-response is non-linear. Addition of 4 mM caffeine during the recovery period has a modest effect on GLA activity in cells treated with 0.3 mM HU. Treatment with 500 μM ddC prior to electroporation doubles DLA activity as compared with untreated cells, but further treatment with 4 mM caffeine or 100 ng/ml trichostatin A (TSA) during the recovery period eliminate the ability of 500 μM ddC to enhance GLA activity.

### Example 9

### Oligonucleotide-Directed Gene Alteration of Pompe Disease Mutation

Pompe disease (MIM 232300), also known as glycogen storage disease II, is an autosomal recessive lysosomal storage disease. Mutations in the gene encoding acid alpha-glucosidase (GAA) are associated with Pompe disease. Studies in Israel show that about 1 in 100 people is a carrier of a disease-causing mutant form of GAA, and that the expected number of individuals born with Pompe disease is 1 on 40,000. Bashan et al., Israel J. Med. Sci. (1988) 24:224-27. The mRNA sequences for GAA are available under accession nos. NM_{—}000152 and NM_{—}199118, the disclosures of which are incorporated herein by reference in their entireties. Oligonucleotides to repair the mutations in GAA are designed by analogy with the correcting oligonucleotides in Example 8. ODSA is performed on cells harboring a mutant GAA variant causing Pompe's disease as in Example 8 to repair the mutant gene.

### Example 10

### Oligonucleotide-Directed Gene Alteration of Gaucher Disease Mutation

Gaucher disease (MIM 230800) is caused by mutations in the gene encoding glucocerebrosidase. Gaucher disease affects approximately 1 in 100,000 persons in the general public, with an incidence of 1 in 450 among Ashkenazic Jews. Mutations in the gene encoding glucocerebrosidase (GBA) are associated with Gaucher disease. The mRNA sequence for GBA is available under accession no. NM_{—}000157, and the human gene sequence is available under accession nos. AF023268 and J03059, the disclosures of which are incorporated herein by reference in their entireties. Oligonucleotides to repair the mutations in GBA are designed by analogy with the correcting oligonucleotides in Example 8. ODSA is performed on cells harboring a mutant GBA variant causing Gaucher disease as in Example 8 to repair the mutant gene.

### Example 11

### Efficient Ex Vivo Gene Repair in Human Blood Cells

Assay system. Oligonucleotide-directed sequence alteration (gene repair) is performed on genetic material in human blood cells using the chromosomal gene encoding the beta subunit of hemoglobin as the target. Two oligonucleotides and a plasmid comprising a mutant copy of the green fluorescent protein (GFP) gene are cointroduced into the cells. The second oligonucleotide is designed to direct an alteration which repairs the mutant GFP resulting in fluorescence. The first oligonucleotide is designed to convert the wild-type allele to the sickle allele. We use first oligonucleotides that correspond in sequence to the wild-type allele at all positions except the single nucleotide position designed to introduce the sickle mutation into the gene. Therefore, these oligonucleotides are identical to the oligonucleotides described in Example 6 and shown in Table 7 except for a single base. For example, we use first oligonucleotides selected from: 5′-C*A*A*CCT CAA ACA GAC ACC ATG GTG CAC CTG ACT CCT GtG GAG AAG TCT GCC GTT ACT GCC CTG TGG GGC AA*G*G*T-3′ (SEQ ID NO.: 7); 5′-A*C*C*TTG CCC CAC AGG GCA GTA ACG GCA GAC TTC TCC aCA GGA GTC AGG TGC ACC ATG GTG TCT GTT TGA GG*T*T*G-3′ (SEQ ID NO.: 8); 5′-ACC TCA AAC AGA CAC CAT GGT GCA CCT GAC TCC TGt GGA GAA GTC TGC CGT TAC TGC CCT GTG GGG CAA GG-3′ (SEQ ID NO.: 9); 5′-G*A*C*ACC ATG GTG CAC CTG ACT CCT GtG GAG AAG TCT GCC GTT ACT GCC*C*T*G-3′ (SEQ ID NO.:10); and 5′-A*C*C*TCA AAC AGA CAC CAT GGT GCA CCT GAC TCC TGt GGA GAA GTC TGC CGT TAC TGC CCT GTG GGG CA*A*G*G-3′ (SEQ ID NO.: 11). The bases in the oligonucleotides that are mismatched to the wild-type allele are shown in lowercase. The oligonucleotides are synthesized with three phosphorothioate linkages on each end (represented with asterisks) or with a single LNA base at each end (bold).

Preparation and treatment of cells. Cells are thawed and electroporated as follows. QBSF-60 medium (Quality Bio) containing 10% FCS (StemCell Technologies) is warmed to 37° C. A vial of frozen G-CSF mobilized peripheral blood CD-34⁺ cells (BioWhittaker) are quickly thawed in a 37° C. water bath, the outside of the tube is wiped with 70% ethanol and about 2 ml (approximately 1×10⁶ cells) of cell suspension is aseptically transferred to a 15 ml or 50 ml conical tube. The vial is rinsed with 1 ml of medium, and which is then added dropwise to the cells, gently swirling the tube every few drops. Medium is slowly added dropwise until the volume is about 5 ml, still gently swirling the conical tube every few drops, and then slowly bringing the volume up to fill the tube by adding 1-2 ml of medium dropwise, swirling after every addition. The cell suspension is centrifuged at 200×g (1500 rpm) for 15 minutes at room temperature. A pipet is used to remove most of the wash to a second tube, leaving a few ml behind to avoid disturbing the cell pellet. The pellet is resuspended in the remaining medium and transferred to a 15 ml conical tube. The original tube is rinsed with 5 ml medium and the wash is added to the cells dropwise, swirling gently after each addition. The cells are recentrifuged at 200×g for 15 minutes.

All but 2 ml of the wash are pipetted off, and the cells are gently resuspended in the remaining medium and counted. The cells are rested at 37° C. and 5% CO₂ for 1 hour and then recounted. Five ml QBSF-60 medium without FCS containing the cytokines flt-3, SCF and TPO at 100 ng/ml final concentration (Stem Cell Technologies) is added, the cells are repelleted at 200×g (1500 rpm for 15 min), and as much liquid volume as possible is gently removed without disturbing the pellet. The cells are resuspended at about 5×10⁵−1×10⁶ cells/ml and transferred to 6-well tissue culture treated dishes. Cells are stimulated for three days with cytokines (QBSF-60 medium, without FCS, containing the cytokines flt-3, SCF and TPO at 100 ng/ml final concentration) and a cell count is performed using trypan blue exclusion staining. The cells are centrifuged at 200×g (1500 rpm) for 15 minutes. The excess volume is removed by pipet and the cells are resuspended in the same medium at 2×10⁶ cells/ml.

The oligonucleotides and the GFP plasmid are electroporated into the cells under square wave conditions as follows. Cell suspension (250 μl), 5 μg GFP plasmid and 30 μg each oligonucleotide are added to a 2 mm gap cuvette and electroporated for one 19 msec pulse at 220 V. Iscove's Medium (Invitrogen™), 10% FCS (StemCell Technologies) (750 μl), cytokines flt-3, SCF, TPO (at 100 ng/ml final concentration), glutamine and penicillin/streptomycin are then added.

Alternatively, 250 μl cell suspension, 250 μl QBSF-60 medium supplemented with flt-3, SCF and TPO and 30 μg oligonucleotide are added to a 4 mm gap cuvette and electroporated for five 19 msec pulses at 220 V with a pulse interval of 1 sec. Iscove's Medium (Invitrogen™) (500 μl), 10% FCS (StemCell Technologies) and the cytokines flt-3, SCF and TPO (at 100 ng/ml final concentration) are then added.

Cells harboring repaired, functional GFP protein are selected using FACS. The sequence of the hemoglobin target in the selected cells is determined by PCR amplification and analysis on the SNapShot™ device using two oligonucleotides: 5′-TTT TTT TTT TTT TTT GAC ACC ATG GTG CAC CTG ACT CCT G-3′ (SEQ ID NO.: 12); and 5′-TTT TTT TTT TTT TTT TTT TTC AGT AAC GGC AGA CTT CTC C-3′ (SEQ ID NO.: 13).

Although a number of embodiments and features are described herein, it will be understood by those skilled in the art that modification and variations of the described embodiments and features may be made without departing from either the spirit of the invention or the scope of the appended claims. Unless specifically stated, no step of the method of this invention requires any particular order of addition of materials, or order of performance of steps. All patents, patent publications, and other published references mentioned herein are incorporated herein by reference in their entireties as if each had been individually and specifically incorporated by reference herein.

An element in a claim is intended to invoke 35 U.S.C. §112 paragraph 6 if and only if it explicitly includes the phrase “means for,” “step for,” or “steps for.” The phrases “step of” and “steps of,” whether included in an element in a claim or in a preamble, are not intended to invoke 35 U.S.C. §112 paragraph 6.

### Sequence Listing

The material contained in the attached compact disk dated May 3, 2005, containing the files labeled 99689-00032US—PatentIn Document having a size of 3 KB and 99689-00032US.ST25—Text Document having a size of 3 KB, is incorporated herein by reference in its entirety.

What is claimed is:

1. A method of increasing the efficiency of oligonucleotide-directed genetic alteration at a specific locus in a target DNA molecule in a population of cells, the method comprising:
treating the population of cells with at least one agent that induces cellular enzymatic activities that promote gene repair or gene editing; and
treating the population of cells with a sequence-altering oligonucleotide.

2. The method of claim 1 wherein the agent is selected from the group consisting of hydroxyurea, thymidine, mimosine, etoposide, methyl methanesulfate, captothecin, dideoxycytidine, and valproic acid.

3. The method of claim 1 further comprising several agents that induce cellular enzymatic activities that promote gene repair or gene editing.

4. The method of claim 3 wherein the agents comprise one or more agents selected from the group consisting of hydroxyurea, thymidine, mimosine, etoposide, methyl methanesulfate, captothecin, dideoxycytidine, valproic acid and combinations thereof.

5. The method of claim 1 wherein the sequence-altering oligonucleotide is complementary to one strand of the target DNA molecule at some nucleotide positions by being non-complementary to the target DNA molecule at the specific locus.

6. The method of claim 1 further comprising treating the population of cells with a vector designed to improve gene editing or repair.

7. The method of claim 1 further comprising treating the population of cells with at least one agent that enriches the population of cells for cells in a particular phase of the cell cycle.

8. The method of claim 7 wherein the particular phase of the cell cycle is S phase.

9. The method of claim 1 further comprising treating the population of cells with at least one agent that reduces the replication rate of the target DNA.

10. The method of claim 1 further comprising inducing DNA damage in the population of cells

11. A method of increasing the efficiency of oligonucleotide-directed genetic alteration at a specific locus in a target DNA molecule in a population of cells, the method comprising:
treating the population of cells with at least one agent that enriches the population of cells for cells in a particular phase of the cell cycle; and
treating the population of cells with a sequence-altering oligonucleotide.

12. The method of claim 11 wherein the particular phase of the cell cycle is S phase.

13. The method of claim 11 wherein the agent is selected from the group consisting of hydroxyurea, thymidine, mimosine, etoposide, methyl methanesulfate, captothecin, dideoxycytidine, and valproic acid.

14. The method of claim 11 further comprising several agents that enrich the population of cells for cells in a particular phase of the cell cycle.

15. The method of claim 14 wherein the particular phase of the cell cycle is S phase.

16. The method of claim 14 wherein the agents comprise one or more agents selected from the group consisting of hydroxyurea, thymidine, mimosine, etoposide, methyl methanesulfate, captothecin, dideoxycytidine, valproic acid and combinations thereof.

17. The method of claim 11 wherein the sequence-altering oligonucleotide is complementary to one strand of the target DNA molecule at some nucleotide positions by being non-complementary to the target DNA molecule at the specific locus.

18. The method of claim 11 further comprising treating the population of cells with a vector designed to improve gene editing or repair.

19. The method of claim 11 further comprising treating the population of cells with at least one agent that reduces the replication rate of the target DNA.

20. The method of claim 11 further comprising inducing DNA damage in the population of cells

21. A method of increasing the efficiency of oligonucleotide-directed genetic alteration at a specific locus in a target DNA molecule in a population of cells, the method comprising:
treating the population of cells with at least one agent that reduces the replication rate of the target DNA; and
treating the population of cells with a sequence-altering oligonucleotide.

22. The method of claim 21 wherein the agent is selected from the group consisting of hydroxyurea, thymidine, mimosine, etoposide, methyl methanesulfate, captothecin, dideoxycytidine, and valproic acid.

23. The method of claim 21 further comprising a plurality agents that reduce the replication rate of the target DNA molecule.

24. The method of claim 23 wherein the agents comprise one or more agents selected from the group consisting of hydroxyurea, thymidine, mimosine, etoposide, methyl methanesulfate, captothecin, dideoxycytidine, valproic acid and combinations thereof.

25. The method of claim 21 wherein the sequence-altering oligonucleotide is complementary to one strand of the target DNA molecule at some nucleotide positions by being non-complementary to the target DNA molecule at the specific locus.

26. The method of claim 21 further comprising treating the population of cells with a vector designed to improve gene editing or repair.

27. The method of claim 21 further comprising inducing DNA damage in the population of cells.
